(19) United States
(12) Patent Application Publication
(10) Pub. No.: US 2010/0254985 A1
(43) Pub. Date: Oct. 7, 2010

(54) Protein Formulations

(75) Inventors: Christian B. ALLAN, Brookeville, MD (US); William LEACH, Columbia, MD (US); Stephen CHANG, New Market, MD (US); Steven BISHOP, Frederick, MD (US)

Correspondence Address: MEDIMMUNE, LLC;Patrick Scott Alban, ONE MEDIMMUNE WAY, GAITHERSBURG, MD, 20878, US

(73) Assignee: MEDIMMUNE, LLC, Gaithersburg, MD (US)

(21) Appl. No.: 12/775,182

(22) Filed: May 6, 2010

Related U.S. Application Data
(63) Continuation of application No. 11/670,786, filed on Feb. 2, 2007.
(60) Provisional application No. 60/764,750, filed on Feb. 3, 2006.
(60) Provisional application No. 60/825,231, filed on Sep. 11, 2006.

(51) Int. Cl. A61K 39/395; A61P 35/00; A61P 31/00; A61P 29/00

(52) U.S. Cl. 424/134.1; 424/133.1

(57) ABSTRACT

The present invention provides formulations of proteins comprising a variant Fc region that improve the stability in part by reducing the propensisty of such molecules to rapidly aggregate. The invention provides both liquid and lyophilized formulations either of which can be utilized to generate a high protein concentration liquid suitable for administration to a subject. The invention further provides methods of utilizing the formulations of the present invention for therapeutic or prophylactic treatment of diseases and disorders or for diagnostic purposes.

### 1. CROSS REFERENCE TO RELATED APPLICATIONS

This application claims the benefit under 35 U.S.C. §119(e) of the following U.S. Provisional Application Nos. 60/764,750 filed Feb. 3, 2006 and 60/825,231 filed Sep. 11, 2006. The priority applications are hereby incorporated by reference herein in their entirety for all purposes.

### 2. FIELD OF THE INVENTION

The present invention provides formulations that improve the stability of proteins, in particular proteins comprising a variant Fc region (e.g., an antibody or Fc fusion protein). In particular, the present invention provides formulations of an Fc variant having a pH of 5.5-8, comprising buffering agent at 1-50 mM and at least one or more of the following, a carbohydrate excipient at about 1-15% weight to volume, a cationic amino acid at about 1-400 mM and an anion at about 1 to 200 mM. The present invention also provides formulations of an Fc variant having a pH of about 5.5 to about 8, comprising an anionic buffer at about 100 mM to about 300 mM and a carbohydrate excipient at about 5-20% weight to volume. The formulations of the present invention include stable liquid formulations and pre lyophilization bulk formulations.

### 3. BACKGROUND OF THE INVENTION

Antibodies are immunological proteins that bind a specific antigen. In most mammals, including humans and mice, antibodies are constructed from paired heavy and light polypeptide chains. Each chain is made up of two distinct regions, referred to as the variable (Fv) and constant (Fc) regions. The light and heavy chain Fv regions contain the antigen binding determinants of the molecule and are responsible for binding the target antigen. The Fc regions define the class (or isotype) of antibody (IgG for example) and are responsible for binding a number of Fc receptors and other Fc ligands, imparting an array of important functional capabilities referred to as effector functions.

An important family of Fc receptors for the IgG class are the Fc gamma receptors (FcγRs). These receptors mediate communication between antibodies and the cellular arm of the immune system (Raghavan et al., 1996, Annu Rev Cell Dev Biol 12:181-220; Ravetch et al., 2001, Annu Rev Immunol 19:275-290). In humans this protein family includes FcγRI (CID64); FcγRII (CD32); and FcγRIII (CID16) (Jefferis et al., 2002, Immunol Lett 82:57-65). These receptors typically have an extracellular domain that mediates binding to Fc, a membrane spanning region, and an intracellular domain that may mediate some signaling event within the cell. Formation of the Fc/FcγR complex recruits effector cells to sites of bound antigen, typically resulting in signaling events within the cells and important subsequent immune responses such as release of inflammation mediators, B cell activation, endocytosis, phagocytosis, and cytotoxic attack. The cell-mediated reaction wherein nonspecific cytotoxic cells that express FcγRs recognize bound antibody on a target cell and subsequently cause lysis of the target cell is referred to as antibody dependent cell-mediated cytotoxicity (ADCC) (Raghavan et al., 1996, Annu Rev Cell Dev Biol 12:181-220; Ghetie et al., 2000, Annu Rev Immunol 18:739-766; Ravetch et al., 2001, Annu Rev Immunol 19:275-290). The Fc region also interacts with the Fc Receptor-neonate (FcRn). This receptor acts as a salvage receptor for antibody recycling (Ghetie et al., 1997, Immunol. Today, 18:592-598) and modulates serum half-life.

Another important Fc ligand is the complement protein C1q. Fc binding to C1q mediates a process called complement dependent cytotoxicity (CDC) (reviewed in Ward et al., 1995, Ther Immunol 2:77-94). C1q is capable of binding six antibodies, although binding to two IgGs is sufficient to activate the complement cascade. C1q forms a complex with the C1r and C1s serine proteases to form the C1 complex of the complement pathway.

Several key features of antibodies including but not limited to, specificity for target, ability to mediate immune effector mechanisms, and long half-life in serum, make antibodies powerful therapeutics. Numerous monoclonal antibodies are currently in development or are being used therapeutically for the treatment of a variety of conditions including cancer. For example etaracizumab (Vitaxin®, MedImmune), a humanized Integrin αᵥβ₃ antibody (e.g., PCT publication WO 2003/075957), Herceptin® (Genentech), a humanized anti-Her2/neu antibody approved to treat breast cancer (e.g., U.S. Pat. No. 5,677,171), CNTO 95 (Centocor), a human Integrin αᵥ antibody (PCT publication WO 02/12501), Rituxan® (IDEC/Genentech/Roche), a chimeric anti-CD20 antibody approved to treat Non-Hodgkin's lymphoma (e.g., U.S. Pat. No. 5,736,137) and Erbitux® (ImClone), a chimeric anti-EGFR antibody (e.g., U.S. Pat. No. 4,943,533). In addition the role of the Fc region in mediating immune effector functions and in stabilizing serum half-life has made it a useful region for generating antibody-like Fc fusion proteins (Chamow et al., 1996, Trends Biotechnol 14:52-60 and Ashkenazi et al., 1997, Curr Opin Immunol 9:195-200). An Fc fusion protein combines the Fc region of an antibody, and thus its favorable effector functions and pharmacokinetics, with the target-binding region of a ligand, receptor, or some other protein domain to mediate target recognition. Fc fusion proteins are also being used therapeutically and/or developed for the treatment of a variety of conditions including arthritis (e.g., Enbrel®, a TNFR-Fc fusion), multiple sclerosis (IFNβ1a-Fc fusion), anemia (EPO-Fc) and hemophilia (FVIII-Fc and FIX-Fc).

It has been shown that altering the binding of the Fc region to its various receptors and ligands can modulate the downstream activities of the Fc region. For example, increasing the binding affinity of the Fc region for FcRn increased the serum half-life of the molecule (Kim et al., Eur. J. Immunol., 24:2429-2434, 1994; Popov et al., Mol. Immunol., 33:493-502, 1996; Ghetie et al., Eur. J. Immunol., 26:690-696, 1996; Junghans et al., Proc. Natl. Acad. Sci. USA, 93:5512-5516, 1996; Israel et al., Immunol., 89:573-578, 1996; and US Patent Publication 2003/0190311). Likewise, increasing the binding affinity of the Fc region for FcγRIIIA increased the ADCC activity of the molecule (Shields et al., 2001, J Biol Chem 276:6591-6604 and Presta et al., 2002, Biochem Soc Trans 30:487-490). Modifications including amino acid deletions, substitutions and additions, as well as changes in the glycosylation of the Fc region have been demonstrated to alter the of the Fc region to its ligands and/or receptors resulting in a concomitant change in effector function (see, e.g., Shields ibid, Presta ibid, and U.S. Patent Publication 2004/0132101). Thus, by modifying the Fc region the therapeutic effectiveness and/or pharmokinetics of Fc containing molecules can be improved. However, as described below, modifications of the Fc region may also result in undesirable characteristics such as a reduction in stability, solubility, or structural integrity. Reductions in stability, solubility or structural integrity present challenges in the development of stable, high concentration formulations for therapeutic or prophylactic administration. Thus, a need exists for formulations which stabilize proteins having a modified Fc region (e.g., an antibody or Fc fusion protein) of interest which are suitable for parenteral administration to a subject.

Citation or discussion of a reference herein shall not be construed as an admission that such is prior art to the present invention.

### 4. SUMMARY OF THE INVENTION

The present invention is based in part on the observation that proteins comprising non naturally occurring Fc regions (e.g., an antibody or Fc fusion protein) are more prone to rapid aggregation as compared to the same protein comprising a naturally occurring Fc region (also referred to herein as a “wild type Fc region”). This aggregation is measure by, for example, size exclusion chromatography (SEC). The present invention is also based in part on the identification of formulations of proteins comprising non naturally occurring Fc regions which increase the stability of said proteins and which are suitable for parenteral administration to a subject. While the formulations of the present invention are particularly useful for stabilizing proteins comprising non naturally occurring Fc regions, it is contemplated that the formulations of the present application could be used to enhance the stability of numerous proteins prone to rapid aggregation. Such formulations offer multiple advantages including less restrictive temperature requirements during the purification/fill/finish process, less stringent or more readily available transportation/storage conditions, and less frequent dosing or smaller dosage amounts in the therapeutic, prophylactic and diagnostic use of such formulations. The invention further provides methods of utilizing the formulations of the present invention for therapeutic or prophylactic treatment of diseases and disorders or for diagnostic purposes.

Proteins comprising non naturally occurring Fc regions (referred to herein as “Fc variant protein(s)”) include, but are not limited to, antibodies and Fc fusion proteins. Non naturally occurring Fc regions (also referred to herein as “variant Fc regions”), include for example, Fc regions comprising non naturally occurring amino acid residues which, may have altered binding properties and/or altered effector function. Non naturally occurring Fc regions can be incorporated into numerous molecules (e.g., antibodies or Fc fusion proteins) to improve their therapeutic effectiveness and/or pharmokinetics.

In one embodiment, the invention provides formulations of Fc variant proteins, which formulations exhibit increased stability due to reduced aggregation of the protein component on storage. In certain embodiments, the formulations of the invention comprise at least 10 mg/mL, or at least 15 mg/mL, 25 mg/mL, 50 mg/mL, 75 mg/mL, 100 mg/mL, 150 mg/mL or 200 mg/mL Fc variant protein.

In one embodiment, the Fc variant protein is an antibody comprising a variant Fc region, wherein said antibody immunospecifically bind an antigen of interest. In a specific embodiment, formulations of an antibody comprising a variant Fc region exhibit increased stability due to reduced aggregation of the antibody on storage. In another embodiment, the Fc variant protein is an Fc fusion protein comprising a variant Fc region or fragment thereof. In another specific embodiment formulations of an Fc fusion protein comprising a variant Fc region exhibit increased stability due to reduced aggregation of the Fc fusion protein component on storage. Such formulations may be used in the diagnostic, therapeutic or prophylactic treatment of diseases and disorders.

In one embodiment, the formulations of the invention comprise an Fc variant protein at about 1 mg/mL to about 200 mg/mL, a buffering agent at about 1 mM to about 100 mM, have a pH of about 5.5 to about 8 and further comprise one or more additional component selected from the group consisting of: a carbohydrate excipient at about 1% to about 15% weight to volume; a cationic amino acid at about 1 mM to about 400 mM; and an anion at about 1 mM to about 200 mM. In another embodiment, the formulations of the invention comprise an Fc variant protein at about 1 mg/mL to about 200 mg/mL, an anionic buffer at about 100 mM to about 300 mM, a carbohydrate excipient at about 5-20% weight to volume and have a pH of about 5.5 to about 8. The formulations of the present invention include stable liquid formulations and pre lyophilization bulk formulations. Optionally, the formulations of the invention may further comprise other common excipients and/or additives such as saccharides, polyols and other amino acids including, but not limited to, glycine, methionine, aspartate and glutamate. Additionally or alternatively, the formulations of the invention may further comprise common excipients and/or additives, such as, but not limited to, solubilizers, diluents, binders, stabilizers, salts, lipophilic solvents, surfactants, chelators, preservatives, or the like.

In certain embodiments, the buffering agent is selected from the group consisting of histidine, phosphate and citrate. In other embodiments the carbohydrate excipient is selected from the group consisting of trehalose, sucrose, mannitol, maltose and raffinose. In still other embodiments the cationic amino acid is selected from the group consisting of lysine, arginine and histidine. In yet other embodiments the anion is selected from the group consisting of citrate, succinate and phosphate.

The present invention encompasses both liquid formulations as well as formulations which are dried by, for example, but not by way of limitation, lyophilization, freeze-drying, spray-drying or air-drying (see, e.g., PCT Publications WO 05/123131; WO 04/058156; WO 03/009817; WO 97/04801 and U.S. Pat. No. 6,165,463). The formulations of the present invention also encompass sterile formulations which may be administered to a subject for therapeutic or prophylactic treatment of diseases and disorders.

In certain embodiments, the formulations of the invention have no more than 10%, or no more than 5%, or no more than 2%, or no more than 1%, or no more than 0.5% aggregate by weight protein at the temperature range of 37° C. to 42° C. for at least 5 days, of 20° C. to 25° C. for at least 30 days, and of 2° C. to 8° C. for at least 90 days or at least 120 days, or at least 180 days, or at least one year, as assessed by sized exclusion chromatograph (SEC) which assays for aggregation.

In one embodiment, the Fc variant protein has enhanced binding to an Fc receptor relative to a protein having the same amino acid sequence except having a wild type Fc region. In a specific embodiment, the Fc variant protein has enhanced binding to the Fc receptor FcγRIIIA. In another specific embodiment, the Fc variant protein has enhanced binding to the Fc receptor FcRn.

In one embodiment, the Fc variant protein has enhanced ADCC activity relative to a protein having the same amino acid sequence except having a wild type Fc region. In another embodiment, the Fc variant protein has enhanced serum half life relative to a protein having the same amino acid sequence except having a wild type Fc region. In still other embodiments, the Fc variant protein has both enhanced ADCC activity and enhanced serum half life relative to a protein having the same amino acid sequence except having a wild type Fc region.

In one embodiment, the present invention provides an Fc variant protein formulation, wherein the Fc region comprises a non naturally occurring amino acid residue at one or more positions selected from the group consisting of 222, 224, 234, 235, 236, 239, 240, 241, 243, 244, 245, 247, 248, 252, 254, 256, 258, 262, 263, 264, 265, 266, 267, 268, 269, 272, 274, 275, 278, 279, 280, 282, 290, 294, 295, 296, 297, 298, 299, 313, 325, 326, 327, 328, 329, 330, 332, 333, 334, 335, 339, 359, 360, 372, 377, 379, 396, 398, 400, 401, 430 and 436, as numbered by the EU index as set forth in Kabat. Optionally, the Fc region may comprise a non naturally occurring amino acid residue at additional and/or alternative positions known to one skilled in the art (see, e.g., U.S. Pat. Nos. 5,624,821; 6,277,375; 6,737,056; PCT Patent Publications WO 01/58957; WO 02/06919; WO 04/016750; WO 04/029207; WO 04/035752 and WO 05/040217).

In a specific embodiment, the present invention provides an Fc variant protein formulation, wherein the Fc region comprises at least one non naturally occurring amino acid residue selected from the group consisting of 222N, 224L, 234D, 234E, 234N, 234Q, 234T, 234H, 234Y, 2341, 234V, 234F, 235A, 235D, 235R, 235W, 235P, 235S, 235N, 235Q, 235T, 235H, 235Y, 2351, 235V, 235F, 236E, 239D, 239E, 239N, 239Q, 239F, 239T, 239H, 239Y, 240I, 240A, 240T, 240M, 241W, 241L, 241Y, 241E, 241R, 243W, 243L 243Y, 243R, 243Q, 244H, 245A, 247V, 247G, 248M, 252Y, 254T, 256E, 258D, 2621, 262A, 262T, 262E, 263I, 263A, 263T, 263M, 264L, 2641, 264W, 264T, 264R, 264F, 264M, 264Y, 264E, 265G, 265N, 265Q, 265Y, 265F, 265V, 265I, 265L, 265H, 265T, 266I, 266A, 266T, 266M, 267Q, 267L, 268D, 268N, 269H, 269Y, 269F, 269R, 296E, 272Y, 274E, 274R, 274T, 275Y, 278T, 279L, 280H, 280Q, 280Y, 282M, 290G, 290S, 290T, 290Y, 294N, 295K, 296Q, 296D, 296N, 296S, 296T, 296L, 296I, 296H, 269G, 297S, 297D, 297E, 298H, 298I, 298T, 298F, 299I, 299L, 299A, 299S, 299V, 299H, 299F, 299E, 300I, 300L, 312A, 313F, 318A, 318V, 320A, 320M, 325Q, 325L, 325I, 325D, 325E, 325A, 325T, 325V, 325H, 327G, 327W, 327N, 327L, 328S, 328M, 328D, 328E, 328N, 328Q, 328F, 328I, 328V, 328T, 328H, 328A, 329F, 329H, 329Q, 330K, 330G, 330T, 330C, 330L, 330Y, 330V, 330I, 330F, 330R, 330H, 332D, 332S, 332W, 332F, 332E, 332N, 332Q, 332T, 332H, 332Y, 332A, 335A, 335T, 335N, 335R, 335Y, 339T, 359A, 360A, 372Y, 377F, 379M, 396H, 396L, 398V, 400P, 401V, 430A, as numbered by the EU index as set forth in Kabat. Optionally, the Fc region may comprise additional and/or alternative non naturally occurring acid residues known to one skilled in the art (see, e.g., U.S. Pat. Nos. 5,624,821; 6,277,375; 6,737,056; PCT Patent Publications WO 01/58957; WO 02/06919; WO 04/016750; WO 04/029207; WO 04/035752 and WO 05/040217). Also encompassed by the present invention are Fc regions which comprise deletions, additions and/or modifications.

In one embodiment, the present invention provides an Fc variant protein formulation, wherein the Fc region comprises at least a non naturally occurring amino acid at one or more positions selected from the group consisting of 252, 254, and 256, as numbered by the EU index as set forth in Kabat. In a specific embodiment, the present invention provides an Fc variant protein formulation, wherein the Fc region comprises at least one non naturally occurring amino acid selected from the group consisting of 252Y, 254T and 256E, as numbered by the EU index as set forth in Kabat.

In another embodiment, the present invention provides an Fc variant protein formulation, wherein the Fc region comprises at least a non naturally occurring amino acid at one or more positions selected from the group consisting of 239, 330 and 332, as numbered by the EU index as set forth in Kabat. In a specific embodiment, the present invention provides an Fc variant protein formulation, wherein the Fc region comprises at least one non naturally occurring amino acid selected from the group consisting of 239D, 330L, 330Y and 332E, as numbered by the EU index as set forth in Kabat. Optionally, the Fc region may further comprise additional non naturally occurring amino acid at one or more positions selected from the group consisting of 252, 254, and 256, as numbered by the EU index as set forth in Kabat. In a specific embodiment, the present invention provides an Fc variant protein formulation, wherein the Fc region comprises at least one non naturally occurring amino acid selected from the group consisting of 239D, 330L, 330Y and 332E, as numbered by the EU index as set forth in Kabat and at least one non naturally occurring amino acid at one or more positions are selected from the group consisting of 252Y, 254T and 256E, as numbered by the EU index as set forth in Kabat.

In other embodiments, the present invention provides an Fc variant protein formulation, wherein the protein comprises one or more engineered glycoforms, i.e., a carbohydrate composition that is covalently attached to the Fc variant protein. Engineered glycoforms may be useful for a variety of purposes, including but not limited to enhancing or reducing effector function. Engineered glycoforms may be generated by any method known to one skilled in the art (see, e.g., U.S. Pat. Nos. 6,602,684; 6,946,292; PCT Publications WO 00/61739; WO 01/292246; WO 02/311140; WO 02/30954; WO 02/079255; WO 00/061739; WO 03/035835 and European Patent Publication EP 01229125).

The present invention encompasses formulations comprising Fc variant proteins derived from virtually any molecule including, but not limited to, proteins, as well as subunits, domains, motifs and epitopes thereof. Non-limiting examples of molecules are, hormones, growthfactors, anti-clotting factors, members of the tumor necrosis factor superfamily, cell surface receptors (e.g., hormone and growth factors receptors), integrin subunits and combinations thereof (e.g. αV, β3, αVβ3, etc), integrin receptors, members of the tyrosine kinse superfamily (e.g., EphA2, EphA4, EphB4, ALK, etc), members of the cluster of differentiation (CD) proteins (e.g., CD19, CD20, CD22, etc), Immunoglobins, cancer antigens, microbial proteins and antibodies and antibody domain fusion proteins (e.g., Fc fusions) that are approved for use, in clinical trials, or in development. In one embodiment, the Fc variant protein compositions comprise an Fc variant protein derived from an antibody that binds to a member of the receptor tyrosine kinase family. In a specific embodiment, the antibody binds EphA2, EphA4, EphB4 or ALK. In another embodiment, the Fc variant protein compositions comprise an Fc variant protein derived from an antibody that binds to an integrin subunit and/combinations thereof. In a specific embodiment, the antibody binds αV, β3, αVβ3.

The Fc variant protein formulations of the invention are useful for the antibody binds diagnosis, prevention, management and treatment of a disease, disorder, infection, including but not limited to inflammatory diseases, autoimmune diseases, bone metabolism related disorders, angiogenic related disorders, infection, and cancer.

### 5. BRIEF DESCRIPTION OF THE FIGURES

FIG. 1. The Nucleotide and Corresponding Amino acid sequence of the variable regions of the heavy (V_{H}) and the light chains (V_{L}) of the anti-EphA2 antibody Medi3 and the anti-Integrin αVβ3 antibody Medi2. Underlined: CDRs (Kabat definition). A) Medi3 V_{H} (SEQ ID NO.: 1-2); B) Medi3 V_{L} (SEQ ID NO.: 3-4); C) Medi2 V_{H} (SEQ ID NO.: 5-6); D) Medi2 V_{L} (SEQ ID NO.: 7-8); SEQ ID NOS. refer to the nucleotide and amino acid sequences, respectively.

FIG. 2. The “V3” Fc variant Increases Non-covalent Aggregation. Panel A is plot of the % monomer present in 100 mg/mL solutions of the anti-EphA2 antibodies Medi3, Medi3-V1, Medi3-V3 and the anti-IntegrinαVβ3 antibody Medi2 over time when formulated in 10 mM histidine buffer, pH 6.0 and stored at 40° C. The percent of monomer in the Medi3-V3 solution decreases by nearly 40% after 14 days, by comparison percent of monomer of the other antibodies, having wild-type Fc regions, dropped by only ˜15% after three months of storage. Panel B is a coomassie stained non-reducing PAGE analysis of two samples of Medi3-V3 having no aggregates (lane 4) or having 30% aggregates (lane 5), neither sample shows any covalent aggregates. Panel C is SEC analysis shows a reduction in % of aggregation of an 80 mg/ml solution of Medi3-V3 in 10 mM Histidine first incubated at 40° C. after each of the following treatments: incubation at 4° C. for 4 and 20 hr (triangles); dilution to 10 mg/ml and incubation at 4° C. for 4 and 20 hr (squares); dilution to 10 mg/ml into 20 mM Citrate buffer and incubation at 4° C. for 4 and 20 hr (closed triangles). Panel D is the percent monomer present in 100 mg/mL solutions of the anti-IntegrinαVβ3 antibodies Medi2 and Medi2-V3 over time when formulated in 10 mM histidine buffer, pH 6.0 and stored at 40° C. The percent monomer drops by less than 10% after two and half months at 40° C. while the Medi2-V3 shows a decrease of ˜22% after less than 1 week at 40° C.

FIG. 3. Fc Variant Regions Have Reduced Tm Values. Panel A) The DSC scans of the wild type Medi3 and the two Fc variants, Medi3-V1 and Medi3-V3 are shown. Arrows indicate the lower temperature melting peak for the C_{H}² domain of the Fc region of Medi3-V1 and Medi3-V3 at 59° C. and 49° C., respectively. The melting temperature of the wild type Medi3 antibody overlaps with the large peak seen for the variable region at 72° C. Panel B) The DSC scans of the wild type Medi2 and Medi2-V3 are shown. The arrows indicate the Tm peaks for the CH2 domain of the Fc region of Medi2-V3. The Tm for Medi3-V3, is ˜47° C. which is very similar to the Tm of ˜49° C. seen for Medi3-V3.

FIG. 4. Aggregation of Medi3-V3 Is Concentration Dependent. A plot of the percent monomer over time for 10, 50 and 100 mg/mL solutions of Medi3-V3 stored in 10 mM histidine buffer, pH 6.0 at 40° C. showing a decrease of 5% at day 37 for the 10 mg/mL solution and a 15% and 37% decrease after just 15 days for the 50 mg/mL and 100 mg/mL solutions, respectively.

FIG. 5. Aggregation of Medi3-V3 Is Temperature Dependent. A plot of the percent monomer over time for a 100 mg/mL solution of Medi3-V3 stored 10 mM histidine buffer, pH 6.0 at 4, 25 and 40° C. showing a decrease of about 37% in 15 days for the solution incubated at 40° C. an a decrease of less than 5% over 30 days for the solutions incubated at 4° C. and 25° C.

FIG. 6. Sucrose, Trehalose And Arginine Stabilize Medi3-V3. A plot of the percent loss in purity for a 7 hour incubation at 40° C. of an 80 mg/mL solution of Medi3-V3 formulated in 10 mM histidine buffer, pH 6.0 plus one of the following excipients, 10% sucrose, 10% trehalose or 200 mM arginine showing that each excipient reduced the percent loss from about 9% in the control (no excipient) to less than 2%.

FIG. 7. Higher Concentrations Of Sugars Stabilize More Effectively. Panel A is a plot of the percent loss in purity for a 24 hour incubation at 40° C. of an 80 mg/mL solution of Medi3-V3 formulated in 10 mM histidine buffer, pH 6.0 plus sugar (sucrose or trehalose) at 0, 1, 5 or 10% as an excipient showing a percent loss in purity of 19%, 16%, 9% and 3%, respectively. The effect of the two sugars was comparable. Panel B is a plot of the percent loss in purity for a 24 hour incubation at 40° C. of a 50 mg/mL solution of Medi3-V3 formulated in 25 mM histidine buffer, pH 6.0 plus sugar (trehalose or mannitol) at 0, 5, 10 or 20% as an excipient showing a percent loss in purity of 8.4%, 4%, 2% and 0.6%, respectively. The effect of the two sugars was comparable.

FIG. 8. Cationic Amino Acids and Anionic Species Are Stabilizing. A plot of the percent loss in purity for a 24 hour incubation at 40° C. of an 80 mg/mL solution of Medi3-V3 formulated in 10 mM histidine buffer, pH 6.0 plus one of the following excipients, arginine, lysine, glycine, cysteine, citrate or DTPA at a final concentration of 0, 50, 200 and/or 400 mM showing that neither cysteine or DTPA were effective at reducing the percent loss at the concentrations tested while the remaining excipients each reduced the percent loss with a relative ranking of citrate>lysine>arginine>glycine.

FIG. 9. Sucrose at 5% And Arginine Are More Effective When Combined. A plot of the percent loss in purity for a 24 hour incubation at 40° C. of an 80 mg/mL solution of Medi3-V3 formulated in 10 mM histidine buffer, pH 6.0 with no excipient, 5% sucrose, 200 mM arginine or both 5% sucrose and 200 mM arginine showing a percent loss of purity of 19%, 9%, 3.5% and 1.5%, respectively.

FIG. 10. Cationic Amino Acids And Anionic Species Are Stabilizing. Panel A is a plot of the percent loss in purity for a 19 hour incubation at 40° C. of an 80 mg/mL solution of Medi3-V3 formulated in 10 mM histidine buffer, pH 6.0 with no excipient, trehalose (10% final), lysine, arginine, histidine, citrate, aspartate, succinate, glutamate, acetate, phosphate, sulfate, serine, phenylalanine, alanine, EDTA or DTPA (each at 50 mM final) showing a percent loss of purity of about 22%, 5.5%, 15.5%, 16%, 16%, 15%, 2%, 10%, 6%, 9%, 11%, <1%, 10%, 17%, 26%, 18%, 20% and 24%, respectively. Panel B is a plot of the percent loss in purity for a 24 hour incubation at 40° C. of a 50 mg/mL solution of Medi3-V3 formulated in 25 mM histidine buffer, pH 6.0 with no excipient, citrate, aspartate, arginine or phosphate at 100 mM, 200 mM or 300 mM. Citrate reduced the percent loss in purity from about 8.4% in the control to ˜1.4% at 100 mM and 0.8% at both 200 mM and 300 mM. Phosphate reduced the percent loss in purity to ˜1.8% at 100 mM and ˜1.0% at both 200 mM and 300 mM while arginine only reduced the percent loss in purity to ˜6.0% at 100 mM and ˜4.8% at both 200 mM and 300 mM.

FIG. 11. Lower Concentrations Of Trehalose And Citrate Are Stabilizing. Panel A is a plot of the percent loss in purity for a 19 hour incubation at 40° C. of an 80 mg/mL solution of Medi3-V3 formulated in 10 mM histidine buffer, pH 6.0 with no excipient, trehalose (10% final), arginine, lysine, citrate (each at 50 mM final), or a combination of trehalose and arginine, lysine or citrate showing that at the concentrations tested only citrate and trehalose combined showed a combinatorial effect reducing the percent loss in purity to just about 1% compared to ˜7% for Trehalose alone or ˜2% for citrate alone. Panel B is a plot of the percent loss in purity for a 1 week incubation at 40° C. of an 50 mg/mL solution of Medi3-V3 formulated with 100, 200 or 300 mM phosphate or citrate in combination with 5, 10 or 20% trehalose or mannitol at pH 6.0 (see Table 3 for details). The 100 mM Citrate, 20% Trehalose; 100 mM Citrate, 20% mannitol and the 300 mM Citrate, 20% Trehalose formulations showed a loss in purity of 1% or less, comparable to that seen for the stable antibody (0.6%). The remaining formulations showed greater then 1% loss in purity.

FIG. 12. Citrate Is A Stronger Stabilizer Than Histidine. A plot of the percent loss in purity for a 19 hour incubation at 40° C. of an 80 mg/mL solution of Medi3-V3 formulated in 10 mM histidine buffer, pH 6.0 with citrate at 25, 50, 100 and 200 mM or formulated in 10 mM citrate buffer, pH 6.0 with histidine at 0, 25, 50 and 100 mM showing that citrate at each concentration tested has a stronger stabilizing effect that histidine.

FIG. 13. pH 5.5 And Above Are Stabilizing. A plot of the percent loss in purity for a 4 hour incubation at 40° C. of an 80 mg/mL solution of Medi3-V3 formulated in 50 mM citrate buffer at pH 3, 3.5, 4, 4.5, 5, 5.5, 6, 6.5, 7 and 8 showing that the percent loss increases dramatically for pH values below 5.5 (from 21% to 90%) and decreases for pH values at or above 5.5 (from 6% to 1%).

FIG. 14. Citrate At Standard Buffer Concentrations Reduces Aggregation. A plot of the percent loss in purity for a 4 hour incubation at 40° C. of an 80 mg/mL solution of Medi3-V3 formulated in 10, 20, 30 or 50 mM citrate at pH ˜5, 6 or 7 showing that at pH 6 and 7 for all concentrations tested the % purity loss was less than ˜6%, at pH 5 citrate was not stabilizing.

FIG. 15. Combinations of Citrate And Certain Amino Acids Or Anionic Species Are Stabilizing. A plot of the percent loss in purity for a 4 hour incubation at 40° C. of an 80 mg/mL solution of Medi3-V3 formulated in 10 mM histidine buffer, pH 6.0 with 20 mM citrate, 35 mM trehalose, arginine, histidine, lysine, aspartate, glutamate, succinate or phosphate alone and in combination with 20 mM citrate showing a 3.3% loss in purity for citrate alone and smaller percent loss in purity (˜0.5% to ˜1.85%) for each combination except histidine.

FIG. 16. Mapping of Combinatorial Formulation Effects. Panels A and B plot the theoretical percent aggregation curves for a 4 hour incubation at 40° C. of an 80 mg/mL solution of Medi3-V3 formulated in 10 mM histidine buffer, pH 6.0, 10% trehalose, citrate at concentrations of 10, 25, 50, 75 and 100 mM and arginine at concentrations of 0, 50, 100, 150 and 200 mM.

FIG. 17. Trehalose Has A Strong Stabilizing Effect At All Citrate Concentrations. Plotted are the theoretical percent aggregation curves for a 4 hour incubation at 40° C. of an 80 mg/mL solution of Medi3-V3 formulated in 10 mM histidine buffer, pH 6.0, 100 mM arginine, citrate at concentrations of 10, 25, 50, 75 and trehalose at concentrations of between 0 and 10%.

FIG. 18. Formulation 1 Significantly Improves Medi3-V3 Stability. A plot of the percent monomer over time for a 10, 25, 50 or 100 mg/mL solution of Medi3-V3 formulated in 50 mM citrate, 10% trehalose, pH 6.5 incubated at 4° C. (Panel A), 25° C. (Panel B) or 40° C. (Panel C) for about 90 days, showing less of a decrease in the percent monomer as compared to the formulation in 10 mM histidine buffer, pH 6.0 (see FIG. 2A).

FIG. 19. Formulation 2 Significantly Improves Medi3-V3 Stability. A plot of the percent monomer over time for a 10, 25, 50 or 100 mg/mL solution of Medi3-V3 formulated in 25 mM citrate, 200 mM arginine, 8% trehalose, pH 6.5 incubated at 4° C. (Panel A), 25° C. (Panel B) or 40° C. (Panel C) for about 90 days, showing less of a decrease in the percent monomer as compared to the formulation in 10 mM histidine buffer, pH 6.0 (see FIG. 2A).

FIG. 20. Two Formulations Significantly Improve Medi2-V3 Stability. A plot of the percent monomer over time for an 80 mg/mL solution of Medi2-V3 formulated in 10 mM histidine buffer, pH 6.0 (control buffer), 50 mM citrate, 10% trehalose, pH 6.0 (Formulation 1′) or 25 mM citrate, 200 mM arginine, 8% trehalose, pH 6.0 (Formulation 2′) incubated at 40° C. for 72 hours showing that both formulation 1′ and 2′ dramatically improve stability.

FIG. 21. Antibodies Recognizing Different Epitopes Having The Same Variant Fc Region Are Stabilized By The Same Formulations. A plot of the percent aggregate present in different formulations of Medi2-V3 and Medi3-V3 at time 0 and after 3 days at 40° C. Both antibodies have ˜23% aggregates after 3 days when formulated in 10 mM H is, pH6 (His). When formulated in 10% trehalose, 50 mM Citrate, pH 6 (Tre/Cit) or 8% trehalose, 25 mM citrate, 200 mM arginine, pH6 (Tre/Cit/Arg) both antibodies have a greatly reduced percent aggregate of ˜4% and ˜8% for the two formulations, respectively.

FIG. 22. Certain Citrate/Trehalose Formulations Without Histidine Stabilize Medi3-V3. The percent aggregate, percent monomer loss, percent fragmentation and the charge variants of Medi3-V3 formulated in four different Citrate/Trehalose formulations (see Table 4) were determined over a 1 month (28 day) incubation at 40° C. Medi2 formulated in 10 mM Histidine, pH 6.0 was used as a control in these studies. Panel A is a plot of the percent aggregate, after 28 days the control had 1.8% aggregate while the Medi3-V3 in Formulation A, B, C and D had 4.18, 2.48, 6.14 and 2.87% aggregate, respectively. Panel B is a plot of the percent monomer loss, after 28 days the control had a monomer loss of 4.6% while the Medi3-V3 in Formulation A, B, C and D had 5.9, 3.61, 8.37, 4.58% monomer loss, respectively. Panel C is a plot of the percent fragment, little difference was seen between Formulations A-D. Panel D is a plot of the charge variants (% prepeak), no difference was seen between Formulations A-D.

FIG. 23. Formulation B Increases the Tm Of The C_{H}² Domain of Medi3-V3. Medi3-V3 was formulated at 0.5 mg/mL in either 10 mM His, pH 6.0 (solid lines) or Formulation B (dotted lines) and the Tm of the C_{H}² domain was determined. Panel A are the DSC scans, the temperature melting peak for the C_{H}² domain in 10 mM His., pH 6 was ˜48° C. and shifted to 55° C. in buffer B. Panel B is a plot of the fluorescence emission intensity at 329 nm vs. temperature, the arrows indicate the transitions which coincide with the melting of the C_{H}² domain. There is about a 10° C. increase in the melting temperature of the C_{H}² domain of Medi3-V3 in Formulation B. Panel C are the plots of the 2^{nd} order derivative UV-V is monitored melting, the arrows indicate the transitions which coincide with the melting of the C_{H}² domain. There is about a 7° C. increase in the melting temperature of the C_{H}² domain of Medi3-V3 in Formulation B.

FIG. 24. Cysteine Enhances Aggregation of Medi3-V3. Panel A is a coomassie stained non-reducing PAGE gel of Medi3-V3 (lanes 1-3) and Medi2 (lanes 4-6) incubated at 37° C. for 16 hours in the presence of 50 mM cysteine (lanes 1 and 4); in the absence of cysteine (lanes 2 and 5) and control samples which were not incubated at 37° C. (lanes 3 and 6). Lane 7 are molecular weight markers, sizes are indicated. Panel B is the SEC analysis of Medi3-V3 incubated at 37° C. for 16 hours in the presence of 50 mM cysteine showing nearly all the antibody is aggregated (bottom); in the absence of cysteine (middle) and control samples which were not incubated at 37° C. (top) both of which show little to no aggregation. Panel C is the SEC analysis of Medi2 incubated at 37° C. for 16 hours in the presence of 50 mM cysteine (bottom); in the absence of cysteine (middle) and control samples which were not incubated at 37° C. (top) each which show only ˜1-1.4% aggregation.

### 6. DETAILED DESCRIPTION

The present invention is based in part on the observation that certain formulations stabilize proteins comprising non naturally occurring Fc regions (referred to herein as “variant Fc regions”) which are more prone to aggregation as compared to the same protein comprising a wild type Fc region. More specifically, the inventors have found that proteins comprising variant Fc regions are more prone to aggregation as compared to the same protein comprising a wild type Fc region when formulated in a variety of buffers such as, for example, 10 mM histidine buffer at pH 6, and that certain formulations reduce the aggregation of proteins comprising variant Fc regions thereby stabilizing them. Accordingly, the present invention provides formulations which increase the stability of proteins comprising variant Fc regions by reducing aggregation of the protein. Proteins comprising a variant Fc region (referred to herein as “Fc variant protein(s)”) include, but are not limited to, antibodies and Fc fusion proteins. “Fc fusion protein” and “Fc fusion” as used herein is a protein wherein one or more polypeptides or small molecules is linked to an Fc region or fragment thereof. Fc fusion is herein meant to be synonymous with the terms “immunoadhesin”, “Ig fusion”, “Ig chimeras”, and “receptor globulin” as used in the prior art (see, e.g., Chamow et al., 1996, Trends Biotechnol 14:52-60; Ashkenazi et al., 1997, Curr Opin Immunol 9:195-200). Variant Fc proteins may be produced “de novo” by combining a protein or fragment thereof (e.g., a variable domain that immunospecifically binds an antigen of interest or the extracellular domain of a receptor of interest) with a variant Fc region, or may be produced by modifying an Fc region-containing protein (e.g., and antibody that binds an antigen of interest or an Fc fusion protein) by introducing one or more non naturally occurring residues into the Fc region.

The formulations provided by the present invention are particularly useful for Fc variant proteins which are more prone to aggregation as compared to the same protein comprising a wild type Fc region. As used herein a protein having the same amino acid sequence as an Fc variant protein except comprising a wild type (WT) Fc region, instead of a variant Fc region, is referred to as a “comparable molecule”.

### 6.1 Fc Variant Protein Formulations

The present invention provides formulations of Fc variant proteins (also referred to herein as “formulations of the invention”), which exhibit increased stability due to reduced aggregation of the Fc variant protein component on storage. The formulations of the invention may comprise any Fc variant protein that has a therapeutic, prophylactic or diagnostic utility. In specific embodiments, the Fc variant protein is one which is more prone to aggregation relative to a comparable molecule, particularly when formulated in 10 mM histidine buffer at pH 6.

The formulations of the invention comprise an Fc variant protein, a buffering agent and further comprise one or more additional components selected from the group consisting of a carbohydrate excipient, a cationic amino acid and an anion. The formulations of the present invention include stable liquid formulations and pre lyophilization bulk formulations.

In certain embodiments, the formulations of the invention comprise an Fc variant protein at about 1 mg/mL to about 200 mg/mL, a buffering agent at about 1 mM to about 100 mM, have a pH of about 5.5 to about 8 and further comprise one or more additional component selected from the group consisting of: a carbohydrate excipient at about 1% to about 15% weight to volume, a cationic amino acid at about 1 mM to about 400 mM, and an anion at about 1 mM to about 200 mM.

In other embodiments, the formulations of the invention comprise an Fc variant protein at about 1 mg/mL to about 200 mg/mL, a buffering agent at about 1 mM to about 100 mM, have a pH of about 5.5 to about 8 and further comprise one or more additional component selected from the group consisting of: a carbohydrate excipient at about 1% to about 20% weight to volume, a cationic amino acid at about 1 mM to about 400 mM, and an anion at about 1 mM to about 200 mM.

In one embodiment, the formulations of the invention comprise an Fc variant protein at about 1 mg/mL to about 200 mg/mL, a buffering agent at about 1 mM to about 100 mM, a carbohydrate excipient at about 1% to about 15% weight to volume and have a pH of about 5.5 to about 8. In certain embodiments, the formulations of the invention comprise an Fc variant protein at about 1 mg/mL to about 200 mg/mL, a buffering agent at about 1 mM to about 100 mM, a carbohydrate excipient at about 1% to about 20% weight to volume and have a pH of about 5.5 to about 8.

In another embodiment, the formulations of the invention comprise an Fc variant protein at about 1 mg/mL to about 200 mg/mL, a buffering agent at about 1 mM to about 100 mM, a cationic amino acid at about 1 mM to about 400 mM, and have a pH of about 5.5 to about 8.

In still another embodiment, the formulations of the invention comprise an Fc variant protein at about 1 mg/mL to about 200 mg/mL, a buffering agent at about 1 mM to about 100 mM, an anion at about 1 mM to about 200 mM and have a pH of about 5.5 to about 8.

In yet another embodiment, the formulations of the invention comprise an Fc variant protein at about 1 mg/mL to about 200 mg/mL, a buffering agent at about 1 mM to about 100 mM, a carbohydrate excipient at about 1% to about 15% weight to volume, a cationic amino acid at about 1 mM to about 400 mM, and have a pH of about 5.5 to about 8. In certain embodiments, the formulations of the invention comprise an Fc variant protein at about 1 mg/mL to about 200 mg/mL, a buffering agent at about 1 mM to about 100 mM, a carbohydrate excipient at about 1% to about 20% weight to volume, a cationic amino acid at about 1 mM to about 400 mM, and have a pH of about 5.5 to about 8.

In other embodiments, the formulations of the invention comprise an Fc variant protein at about 1 mg/mL to about 200 mg/mL, a buffering agent at about 1 mM to about 100 mM, a carbohydrate excipient at about 1% to about 15% weight to volume, a an anion at about 1 mM to about 200 mM and have a pH of about 5.5 to about 8. In certain embodiments, the formulations of the invention comprise an Fc variant protein at about 1 mg/mL to about 200 mg/mL, a buffering agent at about 1 mM to about 100 mM, a carbohydrate excipient at about 1% to about 20% weight to volume, a an anion at about 1 mM to about 200 mM and have a pH of about 5.5 to about 8.

In still other embodiments, the formulations of the invention comprise an Fc variant protein at about 1 mg/mL to about 200 mg/mL, a buffering agent at about 1 mM to about 100 mM and further comprise a cationic amino acid at about 1 mM to about 400 mM, an anion at about 1 mM to about 200 mM and have a pH of about 5.5 to about 8.

In yet other embodiments, the formulations of the invention comprise an Fc variant protein at about 1 mg/mL to about 200 mg/mL, a buffering agent at about 1 mM to about 100 mM, a carbohydrate excipient at about 1% to about 15% weight to volume, a cationic amino acid at about 1 mM to about 400 mM, and an anion at about 1 mM to about 200 mM and have a pH of about 5.5 to about 8. In ceratin embodiments, the formulations of the invention comprise an Fc variant protein at about 1 mg/mL to about 200 mg/mL, a buffering agent at about 1 mM to about 100 mM, a carbohydrate excipient at about 1% to about 20% weight to volume, a cationic amino acid at about 1 mM to about 400 mM, and an anion at about 1 mM to about 200 mM and have a pH of about 5.5 to about 8.

Optionally, the formulations of the invention may further comprise other common auxiliary components, such as, but not limited to, suitable excipients, solubilizers, diluents, binders, stabilizers, salts, lipophilic solvents, surfactants, chelators, preservatives, or the like.

In one embodiment, the formulations of the invention comprise an Fc variant protein at a concentration of least about 1 mg/mL, or at least about 10 mg/mL, or at least about 15 mg/mL, or at least about 25 mg/mL, or at least about 50 mg/mL, or at least about 75 mg/mL, or at least about 100 mg/mL, or at least about 150 mg/mL, or at least about 200 mg/mL or at least about 250 mg/ml, or at least about 300 mg/ml. In specific embodiments the formulations of the invention comprise an Fc variant protein at a concentration of least 1 mg/mL, or at least 10 mg/mL, or at least 15 mg/mL, or at least 25 mg/mL, or at least 50 mg/mL, or at least 75 mg/mL, or at least 100 mg/mL, or at least 150 mg/mL, or at least 200 mg/mL, or at least 250 mg/ml, or at least 300 mg/ml. The formulations of the invention provide exemplary stabilization of Fc variant proteins at concentrations of at least about 25 mg/mL to at least about 200 mg/mL.

The formulations of the invention include a buffering or pH adjusting agent to provide improved pH control. The pH of the formulations of the invention can cover a wide range, such as from about pH 5.5 to about pH 8. In one embodiment the pH ranges from about pH 6 to about pH 8. In another embodiment the pH ranges from about pH 6 to about pH 7. In yet another embodiment the pH ranges from about pH 6.0 to about pH 6.5. In still another embodiment the pH ranges from about pH 6.5 to about 7.0. In a specific embodiment, the pH is about 6.0. In another specific embodiment, the pH is about 6.5. In still other specific embodiments, the pH is 6.0, or 6.1, or 6.2, or 6.3, or 6.4, or 6.5, or 6.6, or 6.7, or 6.8, or 6.9, or 7.0.

Typically, the buffering agent is a salt prepared from an organic or inorganic acid or base. Representative buffering agents include, but are not limited to, organic acid salts such as salts of citric acid, ascorbic acid, gluconic acid, carbonic acid, tartaric acid, succinic acid, acetic acid, or phthalic acid; Tris, tromethamine hydrochloride, or phosphate buffers. In addition, amino acid components can also function in a buffering capacity. Representative amino acid components which may be utilized in the formulations of the invention as buffering agents include, but are not limited to, glycine and histidine. In certain embodiments, the buffering agent is selected from the group consisting of histidine, phosphate and citrate. In a specific embodiment, the buffering agent is citrate. In another specific embodiment, the buffering agent is phosphate. In yet another specific embodiment, the buffering agent is histidine. The purity of the buffering agent should be at least 98%, or at least 99%, or at least 99.5%.

In certain embodiments, formulations of the invention may comprise two cationic amino acids, one as a buffering agent and second as the cationic amino acid component of the formulation. In other embodiments, formulations of the invention may comprise a cationic amino acid at a concentration higher than that typically used for buffering (e.g., higher than about 5 to 50 mM), wherein the cationic amino acid functions both as a buffering agent and the cationic amino acid component of the formulation. It is contemplated that in formulations where the cationic amino acid functions both as a buffering agent and the cationic amino acid component of the formulation the final concentration of the cationic amino acid will be the sum of the concentration of the buffering agent and the concentration of the cationic amino acid. Accordingly, in embodiments, wherein the cationic amino acid functions both as a buffering agent and as the cationic component of the formulation, the cationic amino acid is present at a concentration between about 50 mM to about 500 mM, or between about 100 mM to about 300 mM, or between about 200 mM to about 300 mM, or between about 300 mM to about 400 mM. In certain specific embodiments, wherein the cationic amino acid functions both as a buffering agent and as the cationic component of the formulation, the cationic amino acid is present at a concentration of 50 mM, or of 100 mM, or of 150 mM, or of 200 mM, or of 250 mM, or of 300 mM, or of 350 mM, or of 400 mM.

In certain embodiments, formulations of the invention comprise an Fc variant protein at about 1 mg/mL to about 200 mg/mL, a cationic amino acid buffering agent at about 100 mM to about 500 mM, and a carbohydrate excipient at about 5 to about 20% weight to volume and have a pH of about 5.5 to about 8.

In certain embodiments, formulations of the invention may comprise two anions, one as a buffering agent and second as the anion component of the formulation. In other embodiments, formulations of the invention may comprise an anion at a concentration higher than that typically used for buffering (e.g., higher than about 5 to 50 mM), wherein the anion functions both as a buffering agent and as the anion component of the formulation. It is contemplated that in formulations where the anion functions both as a buffering agent and the anion component of the formulation that the final concentration of the anion will be the sum of the concentration of the buffering agent and the concentration of the anion. Accordingly, in embodiments, wherein the anion functions both as a buffering agent and as the anion component of the formulation, the anion is present at a concentration between about 50 mM to about 300 mM, or between about 100 mM to about 200 mM, or between about 200 mM to about 300 mM. In certain specific embodiments, wherein the anion functions both as a buffering agent and as the anionic component of the formulation, the anion is present at a concentration of 50 mM, or of 100 mM, or of 150 mM, or of 200 mM, or of 250 mM, or of 300 mM.

In other embodiments, formulations of the invention comprise an Fc variant protein at about 1 mg/mL to about 200 mg/mL, an anionic buffering agent at about 100 mM to about 300 mM, and a carbohydrate excipient at about 5-20% weight to volume and have a pH of about 5.5 to about 8. In certain embodiments, formulations of the invention comprise an Fc variant protein at about 50 mg/mL to about 200 mg/mL, an anionic buffering agent at about 100 mM to about 200 mM, and a carbohydrate excipient at about 10 to about 15% weight to volume and have a pH of about 6.0 to about 6.5. In other embodiments, formulations of the invention comprise an Fc variant protein at 50 mg/mL to 200 mg/mL, an anionic buffering agent at 100 mM to 200 mM, and a carbohydrate excipient at 10-15% weight to volume and have a pH of 6.0 to 6.5.

Buffering agents are typically used at concentrations between 1 mM and 200 mM or any range or value therein, depending on the desired ionic strength and the buffering capacity required. The usual concentrations of conventional buffering agents employed in parenteral formulations can be found in: Pharmaceutical Dosage Form: Parenteral Medications, Volume 1, 2^{nd} Edition, Chapter 5, p. 194, De Luca and Boylan, “Formulation of Small Volume Parenterals”, Table 5: Commonly used additives in Parenteral Products. In one embodiment, the buffering agent is at a concentration of about 1 mM, or of about 5 mM, or of about 10 mM, or of about 20 mM, or of about 30 mM, or of about 40 mM, or of about 50 mM, or of about 60 mM, or of about 70 mM, or of about 80 mM, or of about 90 mM, or of about 100 mM. In one embodiment, the buffering agent is at a concentration of 1 mM, or of mM, or of 10 mM, or of 20 mM, or of 30 mM, or of 40 mM, or of 50 mM, or of 60 mM, or of 70 mM, or of 80 mM, or of 90 mM, or of 100 mM. In a specific embodiment, the buffering agent is at a concentration of between about 10 mM and about 50 mM. In another specific embodiment, the buffering agent is at a concentration of between 10 mM and 50 mM.

In certain embodiments, the formulations of the invention comprise a carbohydrate excipient. Carbohydrate excipients can act, e.g., as viscosity enhancing agents, stabilizers, bulking agents, solubilizing agents, and/or the like. Carbohydrate excipients are generally present at between about 1% to about 99% by weight or volume. In one embodiment, the carbohydrate excipient is present at between about 1% to about 20%. In another embodiment, the carbohydrate excipient is present at between about 1% to about 15%. In a specific embodiment, the carbohydrate excipient is present at between about 1% to about 20%, or between about 5% to about 15%, or between about 8% to about 10%, or between about 10% and about 15%, or between about 15% and about 20%. In another specific embodiment, the carbohydrate excipient is present at between 1% to 20%, or between 5% to 15%, or between 8% to 10%, or between 10% and 15%, or between 15% and 20%. In still another specific embodiment, the carbohydrate excipient is present at between about 5% to about 10%. In still another specific embodiment, the carbohydrate excipient is present at between about 10% to about 15%. In yet another specific embodiment, the carbohydrate excipient is present at between about 15% to about 20%. In still other specific embodiments, the carbohydrate excipient is present at 1%, or at 5%, or at 10%, or at 15%, or at 20%.

Carbohydrate excipients suitable for use in the formulations of the invention include, for example, monosaccharides such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol) and the like. In one embodiment, the carbohydrate excipients for use in the present invention are selected from the group consisting of, sucrose, trehalose, lactose, mannitol, and raffinose. In a specific embodiment, the carbohydrate excipient is sucrose. In another specific embodiment, the carbohydrate excipient is trehalose. In yet another specific embodiment, the carbohydrate excipient is mannitol. In still another specific embodiment, the carbohydrate excipient is raffinose. The purity of the carbohydrate excipient should be at least 98%, or at least 99%, or at least 99.5%.

In certain embodiments, the formulations of the invention comprise a cationic amino acid. In one embodiment, the cationic amino acid is present at between about 1 mM to about 400 mM. In a specific embodiment, the cationic amino acid is present at between about 25 mM to about 200 mM. In another specific embodiment, the cationic amino acid is present at a concentration of at least 10 mM, or at least 20 mM, or at least 30 mM, or at least 40 mM, or at least 50 mM, or at least 75 mM, or at least 100 mM, or at least 150 mM, or at least 200 mM, or at least 250 mM, or at least 300 mM, or at least 350 mM, or at least 400 mM. Cationic amino acids are known to one skilled in the art, and may be naturally occurring or modified amino acids. Cationic amino acids which may be utilized for the formulations of the present invention include, but are not limited to, L-lysine, D-lysine, L-dimethylysine, D-dimethylysine, L-histidine, D-histidine, L-ornithine, D-ornithine, L-arginine, D-arginine, L-homoarginine, D-homoarginine, L-norarginine, D-norarginine, 2,4-diaminobutyric acid, homolysine and p-lysine. In one embodiment, the formulations of the invention comprise the cationic amino acid lysine. In another embodiment, the formulations of the invention comprise the cationic amino acid arginine. In still another embodiment, the formulations of the invention comprise the cationic amino acid histidine. It is contemplated formulations of the invention may comprise two cationic amino acids, one as a buffering agent and second as the cationic amino acid component of the formulation. As noted above, a cationic amino acid may be present at higher concentration and function both as a buffering agent and as the cationic amino acid component of the formulation. The purity of the cationic amino acid should be at least 98%, or at least 99%, or at least 99.5%.

In certain embodiments, the formulations of the invention comprise an anion. In one embodiment, the anion is present at between about 1 mM to about 200 mM. In another specific embodiment, the anion is present at a concentration of at least 10 mM, or at least 20 mM, or at least 30 mM, or at least 40 mM, or at least 50 mM, or at least 75 mM, or at least 100 mM, or at least 150 mM, or at least 200 mM. Non-limiting examples of anions are nitrate, nitrite, chloride, cyanide, bromide, iodide, carbonate, bicarbonate, sulfate, phosphate, acetate, citrate and succinate. In addition, a number of naturally occurring and modified amino acids may be used as anions including, but not limited to, L-aspartate, D-aspartate, L-glutamate, D-glutamate γ-carboxyglutamate. In one embodiment, the formulations of the invention comprise the anion citrate. In another embodiment, the formulations of the invention comprise the anion succinate. In still another embodiment, the formulations of the invention comprise the anion phosphate. It is contemplated formulations of the invention may comprise two anions, one as a buffering agent and second as the anion component of the formulation. As noted above, a cationic amino acid may be present at higher concentration and function both as a buffering agent and as the anion component of the formulation. The purity of the anion should be at least 98%, or at least 99%, or at least 99.5%.

In certain embodiments, the formulations of the invention comprise an amino acid. In one embodiment, the amino acid is present at between about 1 mM to about 200 mM. In another specific embodiment, the amino acid is present at a concentration of at least 10 mM, or at least 20 mM, or at least 30 mM, or at least 40 mM, or at least 50 mM, or at least 75 mM, or at least 100 mM, or at least 150 mM, or at least 200 mM. Non-limiting examples of amino acids include alanine, arginine, asparagines, aspartic acid, cysteine, glutamine, glutamic acid, glycine, histidine, isoleucine, leucine, lysine, methionine, phenylalanine, proline, serine, threonine, tryptophan, tyrosine, and valine. In addition, a large number modified amino acids may be used. It is contemplated formulations of the invention may comprise two amino acids, for example, one as the anion component of the formulation and a second as an excipient. Alternatively formulations of the invention may comprise two amino acids, wherein one is the cationic amino acid component of the formulation and the second is the excipient. It is contemplated that a single amino acid may be present at higher concentration and function as both the excipient and as the cationic amino acid and/or anionic component of the formulation. The purity of the amino acid should be at least 98%, or at least 99%, or at least 99.5%.

In certain embodiments, the formulations of the invention do not comprise cysteine as an excipient and/or additive. In certain other embodiments, the formulations of the invention do not comprise methionine as an excipient and/or additive.

Optionally, the formulations of the invention may further comprise other common excipients and/or additives including, but not limited to, diluents, binders, stabilizers, buffers, salts, lipophilic solvents, preservatives, adjuvants, surfactants or the like. Pharmaceutically acceptable excipients and/or additives are preferred for use in the formulations of the invention. Commonly used excipients/additives, such as pharmaceutically acceptable surfactants like polysorbate, Tween 20 (polyoxyethylene (20) sorbitan monolaurate), Tween 40 (polyoxyethylene (20) sorbitan monopalmitate), Tween 80 (polyoxyethylene (20) sorbitan monooleate), Pluronic F68 (polyoxyethylene polyoxypropylene block copolymers), and PEG (polyethylene glycol) or surfactants such as polysorbate 20 or 80 or poloxamer 184 or 188, Pluronic® polyls, other block co-polymers and chelators such as EDTA, DTPA or EGTA can optionally be added to the formulations of the invention to reduce aggregation. These additives are particularly useful if a pump or plastic container is used to administer the formulation. The presence of pharmaceutically acceptable surfactant mitigates the propensity for the protein to aggregate. In a specific embodiment, the formulations of the invention comprise a polysorbate which is at a concentration ranging from between about 0.001% to about 1%, or about 0.001% to about 0.1%, or about 0.01% to about 0.1%. In other specific embodiments, the formulations of the invention comprise a polysorbate which is at a concentration of 0.001%, or 0.002%, or 0.003%, or 0.004%, or 0.005%, or 0.006%, or 0.007%, or 0.008%, or 0.009%, or 0.01%, or 0.015%, or 0.02%. In another specific embodiment, the polysorbate is polysorbate-80.

Preservatives, such as phenol, m-cresol, p-cresol, o-cresol, chlorocresol, benzyl alcohol, phenylmercuric nitrite, phenoxyethanol, formaldehyde, chlorobutanol, magnesium chloride (e.g., hexahydrate), alkylparaben (methyl, ethyl, propyl, butyl and the like), benzalkonium chloride, benzethonium chloride, sodium dehydroacetate and thimerosal, or mixtures thereof can optionally be added to the formulations of the invention at any suitable concentration such as between about 0.001% to about 5%, or any range or value therein. The concentration of preservative used in the formulations of the invention is a concentration sufficient to yield an microbial effect. Such concentrations are dependent on the preservative selected and are readily determined by the skilled artisan.

Other contemplated excipients/additives, which may be utilized in the formulations of the invention include, for example, flavoring agents, antimicrobial agents, sweeteners, antioxidants, antistatic agents, lipids such as phospholipids or fatty acids, steroids such as cholesterol, protein excipients such as serum albumin (human serum albumin (HSA), recombinant human albumin (rHA)), gelatin, casein, salt-forming counterions such as sodium and the like. These and additional known pharmaceutical excipients and/or additives suitable for use in the formulations of the invention are known in the art, e.g., as listed in “Remington: The Science & Practice of Pharmacy”, 21^{st} ed., Lippincott Williams & Wilkins, (2005), and in the “Physician's Desk Reference”, 60^{th} ed., Medical Economics, Montvale, N.J. (2005). Pharmaceutically acceptable carriers can be routinely selected that are suitable for the mode of administration, solubility and/or stability of Fc variant protein as well known in the art or as described herein.

It will be understood by one skilled in the art that the formulations of the invention may be isotonic with human blood, that is the formulations of the invention have essentially the same osmotic pressure as human blood. Such isotonic formulations will generally have an osmotic pressure from about 250 mOSm to about 350 mOSm. Isotonicity can be measured by, for example, using a vapor pressure or ice-freezing type osmometer. In certain embodiments, the formulations of the present invention have an osmotic pressure from about 100 mOSm to about 1200 mOSm, or from about 200 mOSm to about 1000 mOSm, or from about 200 mOSm to about 800 mOSm, or from about 200 mOSm to about 600 mOSm, or from about 250 mOSm to about 500 mOSm, or from about 250 mOSm to about 400 mOSm, or from about 250 mOSm to about 350 mOSm. Accordingly, the concentration of the components of the formulations of the invention are adjusted depending on the desired isotonicity of the final formulation (e.g., of the final liquid or reconstituted formulation). For example, the ratio of the carbohydrate excipient to Fc variant protein may be adjusted according to methods known in the art (e.g., U.S. Pat. No. 6,685,940). In certain embodiments, the molar ratio of the carbohydrate excipient to Fc variant protein may be from about 100 moles to about 1000 moles of carbohydrate excipient to about 1 mole of Fc variant protein, or from about 200 moles to about 6000 moles of carbohydrate excipient to about 1 mole of Fc variant protein, or from about 100 moles to about 510 moles of carbohydrate excipient to about 1 mole of Fc variant protein, or from about 100 moles to about 600 moles of carbohydrate excipient to about 1 mole of Fc variant protein.

In one embodiment the formulations of the invention are pyrogen-free formulations which are substantially free of endotoxins and/or related pyrogenic substances. Endotoxins include toxins that are confined inside a microorganism and are released only when the microorganisms are broken down or die. Pyrogenic substances also include fever-inducing, thermostable substances (glycoproteins) from the outer membrane of bacteria and other microorganisms. Both of these substances can cause fever, hypotension and shock if administered to humans. Due to the potential harmful effects, even low amounts of endotoxins must be removed from intravenously administered pharmaceutical drug solutions. The Food & Drug Administration (“FDA”) has set an upper limit of 5 endotoxin units (EU) per dose per kilogram body weight in a single one hour period for intravenous drug applications (The United States Pharmacopeial Convention, Pharmacopeial Forum 26 (1):223 (2000)). When therapeutic proteins are administered in amounts of several hundred or thousand milligrams per kilogram body weight, as can be the case with antibodies or Fc fusion proteins, even trace amounts of harmful and dangerous endotoxin must be removed. In certain specific embodiments, the endotoxin and pyrogen levels in the composition are less then 10 EU/mg, or less then 5 EU/mg, or less then 1 EU/mg, or less then 0.1 EU/mg, or less then 0.01 EU/mg, or less then 0.001 EU/mg.

When used for in vivo administration, the formulations of the invention should be sterile. The formulations of the invention may be sterilized by various sterilization methods, including sterile filtration, radiation, etc. In one embodiment, the Fc variant protein formulation is filter-sterilized with a presterilized 0.22-micron filter. Sterile compositions for injection can be formulated according to conventional pharmaceutical practice as described in “Remington: The Science & Practice of Pharmacy”, 21^{st} ed., Lippincott Williams & Wilkins, (2005). Formulations comprising Fc variant proteins, such as those disclosed herein, ordinarily will be stored in lyophilized form or in solution. It is contemplated that sterile compositions comprising Fc variant proteins are placed into a container having a sterile access port, for example, an intravenous solution bag or vial having an adapter that allows retrieval of the formulation, such as a stopper pierceable by a hypodermic injection needle.

The present invention encompasses both liquid formulations as well as formulations which are dried. In certain embodiments, the formulations are liquid formulations. The liquid formulations of the present invention can be prepared as unit dosage forms by preparing a vial containing an aliquot of the liquid formulation for a one-time use. For example, a unit dosage per vial may contain 1 ml, 2 ml, 3 ml, 4 ml, 5 ml, 6 ml, 7 ml, 8 ml, 9 ml, 10 ml, 15 ml, 20 ml or any range or value therein, of different concentrations of an Fc variant protein ranging from about 10 mg/ml to about 200 mg/ml. If necessary, these preparations can be adjusted to a desired concentration by adding a sterile diluent to each vial.

In other embodiments, the formulations are dried formulations which are reconstituted prior to administration. For the preparation of dried formulations the Fc variant protein is prepared as a “pre-lyophilized formulation” comprising one or more component disclosed herein, wherein the amount of protein and other formulation components (e.g., excipients and/or additives) is determined, taking into account the desired dose volumes, mode(s) of administration, etc., and the resulting formulation is dried. In one embodiment, the pre-lyophilized formulation is prepared such that upon reconstitution the resulting reconstituted formulation will comprise an Fc variant protein at about 1 mg/mL to about 200 mg/mL, a buffering agent at about 1 mM to about 100 mM, have a pH of about 5.5 to about 8 and further comprise one or more additional component selected from the group consisting of, a carbohydrate excipient at about 1% to about 15% weight to volume, a cationic amino acid at about 1 mM to about 400 mM, and an anion at about 1 mM to about 200 mM. In another embodiment, the pre-lyophilized formulation is prepared such that upon reconstitution the resulting reconstituted formulation further comprises a surfactant at about 0.001% to about 0.05%.

It is contemplated that any of the formulations of the present invention may be utilized as a pre-lyophilized formulation, also referred to here in as a “pre-lyophilization bulk formulation”.

In certain embodiments, a formulation of the invention is a pre-lyophilized bulk formulation comprising an Fc variant protein at about 20 mg/mL to about 100 mg/mL, a buffering agent at about 1 mM to about 25 mM, having a pH of about 5.5 to about 6.5 and further comprising one or more additional components selected from the group consisting of, a carbohydrate excipient at about 1% to about 10% weight to volume, a cationic amino acid at about 50 mM to about 200 mM, and an anion at about 50 mM to about 200 mM and a surfactant at about 0.001% to about 0.05%. In a specific embodiment, a formulation of the invention is a pre-lyophilized bulk formulation comprising an Fc variant protein at 20 mg/mL to 100 mg/mL, a buffering agent at 1 mM to 25 mM, having a pH of 5.5 to 6.5 and further comprising one or more additional components selected from the group consisting of, a carbohydrate excipient at 1% to 10% weight to volume, a cationic amino acid at 50 mM to 200 mM, and an anion at 50 mM to 200 mM and a surfactant at 0.001% to 0.05%.

Specific methods to produce dried forms of liquid formulations are well-characterized in the art, for example, but not by way of limitation, lyophilization, freeze-drying, spray-drying or air-drying (see, e.g., PCT Publication WO 05/123131; WO 04/058156, WO 03/009817; WO 97/04801 and U.S. Pat. No. 6,165,463). In one embodiment, the ingredients of formulation of the invention are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

### 6.2 Stability of Fc Variant Protein Formulations

In certain embodiments, the formulations of the invention reduce the aggregation of an Fc variant compared to the aggregation when the same Fc variant is formulated in 10 mM Histidine pH 6.0. In a specific embodiment, the formulations of the invention reduce the aggregation of an Fc variant by at least 5%, or at least 10% or at least 20%, or at least 30%, or at least 40%, or at least 50%, or at least 60%, or at least 70%, or at least 80%, or at least 90%, or at least 100%, compared to the aggregation when the same Fc variant is formulated in 10 mM Histidine pH 6.0. In another specific embodiment, the formulations of the invention reduce the aggregation of an Fc variant by at least 2 fold, or least 5 fold, or least 10 fold, or least 20 fold, or least 30 fold, or least 40 fold, or least 50 fold, or least 60 fold, or least 70 fold, or least 80 fold, or least 90 fold, or least 100 fold, or least 200 fold, or least 500 fold, compared to the aggregation when the same Fc variant is formulated in 10 mM Histidine pH 6.0.

In certain embodiments, the formulations of the invention maintain improved aggregation profiles upon storage, for example, for extended periods (for example, but not limited to 1 week, 1 month, 6 months, 1 year, 2 years, 3 years or 5 years) at room temperature or 4° C. or for periods (such as, but not limited to 1 week, 2 weeks, 3 weeks, 1 month, 2 months, 3 months, or 6 months) at elevated temperatures such as 38° C.-42° C. In certain embodiments, the formulations maintain improved aggregation profiles upon storage while exposed to light or stored in the dark in a variety of humidity conditions including but not limited to a relative humidity of up to 10%, or up to 20%, or up to 30%, or up to 40%, or up to 50%, or up to 60%, or up to 70%, or up to 80%, or up to 90%, or up to 100%. It will be understood in the art that the term “ambient” conditions generally refers to temperatures of about 20° C. at a relative humidity of between 10% and 60% with exposure to light. Similarly, temperatures between about 2° C. and about 8° C. at a relative humidity of less then about 10% are collectively referred to as “4° C.” or “5° C.”, temperatures between about 23° C. and about 27° C. at a relative humidity of about 60% are collectively referred to as “25° C.” and temperatures between about 38° C. and about 42° C. at a relative humidity of about 75% are collectively referred to as “40° C.”

In specific embodiments, the formulations of the invention have no more than 20%, or no more than 10%, or no more than 5%, or no more than 2%, or no more than 1%, or no more than 0.5%, or no more than 0.4%, or no more than 0.2%, or no more than 0.1%, or less than 0.1% aggregate, relative to total protein at the temperature range of 37° C. to 42° C. for at least 5 days, of 20° C. to 25° C. for at least 30 days, and of 2° C. to 8° C. for at least 90 days, or at least 120 days, or at least 180 days, or at least one year, as assessed by sized exclusion chromatograph (SEC) or similar assays useful for determining the degree of aggregation in a sample. In other specific embodiments, the formulations of the invention have no more than about 20%, or no more than about 10%, or no more than about 5%, or no more than about 2%, or no more than about 1%, or no more than about 0.5%, or no more than about 0.4%, or no more than about 0.2%, or no more than about 0.1%, or less than about 0.1% aggregate, relative to total protein at the temperature range of 38° C. to 42° C. for at least 5 days, of 23° C. to 27° C. for at least 30 days, and of 2° C. to 8° C. for at least 90 days, as assessed by sized exclusion chromatograph (SEC) or similar assays useful for determining the degree of aggregation in a sample.

Namely, the formulations of the invention have low to undetectable levels of aggregation, as defined herein, after the storage for the defined periods as set forth above. In one embodiment, no more than 20%, no more than 10%, no more than 5%, no more than 4%, no more than 3%, no more than 2%, no more than 1%, or no more than 0.5%, or no more than 0.4%, or no more than 0.2%, or no more than 0.1% (but in certain embodiments, at least 0.1%) of the Fc variant protein forms an aggregate as measured by SEC or similar assays useful for determining the degree of aggregation in a sample after the storage for the defined periods as set forth above.

Furthermore, formulations of the present invention exhibit almost no loss in biological activities of the Fc variant protein during the prolonged storage under the condition described above. The formulations of the present invention retain after the storage for the above-defined periods more than 80%, more than 85%, more than 90%, more than 95%, more than 98%, more than 99%, or more than 99.5% of the initial biological activities of the formulation prior to the storage.

It is contemplated that during storage, the formulations exhibit constant aggregation rates at temperatures, such as, but not limited to, 0-4° C., 2-8° C., 10-15° C., 20-24° C., 23-27° C., room temperature, or elevated temperatures 38-42° C., and extended periods, such as, but not limited to, one week, two weeks, one month, six months, one year, three years or five years. Thus, in one embodiment, an Fc variant protein formulation will increase in aggregate percentage relative to total protein, by not more than 1%/month to 10%/month at 38-42° C., or by not more than 0.2%/month to 1.0%/month at 20-24° C., or by not more than 0.2%/month at 4° C. (i.e. 2-8° C.).

In certain embodiments, after storage at 4° C. for at least one month, the formulations of the invention comprise (or consists of as the aggregate fraction) a particle profile of less than about 3.4 E+5 particles/ml of diameter 2-4 μm, less than about 4.0 E+4 particles/ml of diameter 4-10 μm, less than about 4.2 E+3 particles/ml of diameter 10-20 μm, less than about 5.0 E+2 particles/ml of diameter 20-30 μm, less than about 7.5 E+1 particles/ml of diameter 30-40 μm, and less than about 9.4 particles/ml of diameter 40-60 μm as determined by a particle multisizer. In certain embodiments, the formulations of the invention contain no detectable particles greater than 40 μm, or greater than 30 μm.

While the formulations of the present invention are particularly useful for stabilizing an Fc variant protein, it is contemplated that the formulations of the present application could be used to enhance the stability of numerous proteins prone to rapid aggregation. Accordingly, in one embodiment, the formulations of the invention reduce the aggregation of a protein prone to aggregation. In a specific embodiment, the formulations of the invention reduce the aggregation of a protein prone to aggregation by at least 5%, or at least 10% or at least 20%, or at least 30%, or at least 40%, or at least 50%, or at least 60%, or at least 70%, or at least 80%, or at least 90%, or at least 100% as compared to the same concentration of the protein in a formulation in which it is know to aggregate.

Furthermore, it is known in the art, that numerous proteins are more prone to aggregation when formulated at higher concentrations. Accordingly, the formulations of the present invention can be used to formulate high concentration formulations of a protein which is known to aggregate at high concentrations. In one specific embodiment, the formulations of the invention reduce the aggregation at high concentrations (e.g, 20 mg/mL or higher) of a protein prone to aggregation at high concentration to that of the protein formulated in another buffer at lower concentrations (e.g., less than 20 mg/mL). In a specific embodiment, the formulations of the invention allow a protein more prone to aggregation at high concentrations to be formulated at a concentration of at least 20 mg/mL, or at least 30 mg/mL, or at least 40 mg/mL, or at least 50 mg/mL, or at least 60 mg/mL, or at least 70 mg/mL, or at least 80 mg/mL, or at least 90 mg/mL, or at least 100 mg/mL, or at least 200 mg/mL, wherein no more than 20%, no more than 10%, no more than 5%, no more than 4%, no more than 3%, no more than 2%, no more than 1%, or no more than 0.5%, or no more than 0.4%, or no more than 0.2%, or no more than 0.1% of said protein forms an aggregate.

Numerous methods useful for determining the degree of aggregation, and/or types and/or sizes of aggregates present in a protein formulation (e.g., Fc variant protein formulation of the invention) are known in the art, including but not limited to, size exclusion chromatography (SEC), high performance size exclusion chromatography (HPSEC), static light scattering (SLS), Fourier Transform Infrared Spectroscopy (FTIR), circular dichroism (CD), urea-induced protein unfolding techniques, intrinsic tryptophan fluorescence, differential scanning calorimetry, and 1-anilino-8-naphthalenesulfonic acid (ANS) protein binding techniques. For example, size exclusion chromatography (SEC) may be performed to separate molecules on the basis of their size, by passing the molecules over a column packed with the appropriate resin, the larger molecules (e.g. aggregates) will elute before smaller molecules (e.g. monomers). The molecules are generally detected by UV absorbance at 280 nm and may be collected for further characterization. High pressure liquid chromatographic columns are often utilized for SEC analysis (HP-SEC). Specific SEC methods are detailed in the section entitled “Examples” infra. Alternatively, Analytical ultracentrifugation (AUC) may be utilized. AUC is an orthogonal technique which determines the sedimentation coefficients (reported in Svedberg, S) of macromolecules in a liquid sample. Like SEC, AUC is capable of separating and detecting antibody fragments/aggregates from monomers and is further able to provide information on molecular mass. Protein aggregation in the formulations may also be characterized by particle counter analysis using a coulter counter or by turbidity measurements using a turbidimeter. Turbidity is a measure of the amount by which the particles in a solution scatter light and, thus, may be used as a general indicator of protein aggregation. In addition, non-reducing polyacrylamide gel electrophoresis (PAGE) or capillary gel electrophoresis (CGE) may be used to characterize the aggregation and/or fragmentation state of the Fc variant proteins in the formulations of the invention. Specific examples of PAGE and CEG methods are detailed in the section entitled “Examples” infra.

### 6.3 Variant Fc Regions

The present invention provides formulation of proteins comprising a variant Fc region. That is, a non naturally occurring Fc region, for example an Fc region comprising one or more non naturally occurring amino acid residues. Also encompassed by the variant Fc regions of present invention are Fc regions which comprise amino acid deletions, additions and/or modifications.

It will be understood that Fc region as used herein includes the polypeptides comprising the constant region of an antibody excluding the first constant region immunoglobulin domain. Thus Fc refers to the last two constant region immunoglobulin domains of IgA, IgD, and IgG, and the last three constant region immunoglobulin domains of IgE and IgM, and the flexible hinge N-terminal to these domains. For IgA and IgM Fc may include the J chain. For IgG, Fc comprises immunoglobulin domains Cgamma2 and Cgamma3 (Cγ2 and Cγ3) and the hinge between Cgamma1 (Cγ1) and Cgamma2 (Cγ2). Although the boundaries of the Fc region may vary, the human IgG heavy chain Fc region is usually defined to comprise residues C226 or P230 to its carboxyl-terminus, wherein the numbering is according to the EU index as in Kabat et al. (1991, NIH Publication 91-3242, National Technical Information Service, Springfield, Va.). The “EU index as set forth in Kabat” refers to the residue numbering of the human IgG1 EU antibody as described in Kabat et al. supra. Fc may refer to this region in isolation, or this region in the context of an antibody, antibody fragment, or Fc fusion protein. An Fc variant protein may be an antibody, Fc fusion, or any protein or protein domain that comprises an Fc region. Particularly preferred are proteins comprising variant Fc regions, which are non naturally occurring variants of an Fc. Note: Polymorphisms have been observed at a number of Fc positions, including but not limited to Kabat 270, 272, 312, 315, 356, and 358, and thus slight differences between the presented sequence and sequences in the prior art may exist.

The present invention encompasses Fc variant proteins which have altered binding properties for an Fc ligand (e.g., an Fc receptor, C1q) relative to a comparable molecule (e.g., a protein having the same amino acid sequence except having a wild type Fc region). Examples of binding properties include but are not limited to, binding specificity, equilibrium dissociation constant (K_{D}), dissociation and association rates (K_{off} and Kₒₙ respectively), binding affinity and/or avidity. It is generally understood that a binding molecule (e.g., a Fc variant protein such as an antibody) with a low K_{D} is preferable to a binding molecule with a high K_{D}. However, in some instances the value of the kₒₙ or k_{off} may be more relevant than the value of the K_{D}. One skilled in the art can determine which kinetic parameter is most important for a given antibody application.

The affinities and binding properties of an Fc domain for its ligand, may be determined by a variety of in vitro assay methods (biochemical or immunological based assays) known in the art for determining Fc-FcγR interactions, i.e., specific binding of an Fc region to an FcγR including but not limited to, equilibrium methods (e.g., enzyme-linked immunoabsorbent assay (ELISA); see Example 3, or radioimmunoassay (RIA)), or kinetics (e.g., BIACORE® analysis), and other methods such as indirect binding assays, competitive inhibition assays, fluorescence resonance energy transfer (FRET), gel electrophoresis and chromatography (e.g., gel filtration). These and other methods may utilize a label on one or more of the components being examined and/or employ a variety of detection methods including but not limited to chromogenic, fluorescent, luminescent, or isotopic labels. A detailed description of binding affinities and kinetics can be found in Paul, W. E., ed., Fundamental Immunology, 4^{th} Ed., Lippincott-Raven, Philadelphia (1999), which focuses on antibody-immunogen interactions.

In one embodiment, the Fc variant protein has enhanced binding to one or more Fc ligand relative to a comparable molecule. In another embodiment, the Fc variant protein has an affinity for an Fc ligand that is at least 2 fold, or at least 3 fold, or at least 5 fold, or at least 7 fold, or a least 10 fold, or at least 20 fold, or at least 30 fold, or at least 40 fold, or at least 50 fold, or at least 60 fold, or at least 70 fold, or at least 80 fold, or at least 90 fold, or at least 100 fold, or at least 200 fold greater than that of a comparable molecule. In a specific embodiment, the Fc variant protein has enhanced binding to an Fc receptor. In another specific embodiment, the Fc variant protein has enhanced binding to the Fc receptor FcγRIIIA. In still another specific embodiment, the Fc variant protein has enhanced binding to the Fc receptor FcRn. In yet another specific embodiment, the Fc variant protein has enhanced binding to C1q relative to a comparable molecule.

The serum half-life of proteins comprising Fc regions may be increased by increasing the binding affinity of the Fc region for FcRn. In one embodiment, the Fc variant protein has enhanced serum half life relative to comparable molecule.

“Antibody-dependent cell-mediated cytotoxicity” or “ADCC” refers to a form of cytotoxicity in which secreted Ig bound onto Fc receptors (FcRs) present on certain cytotoxic cells (e.g., Natural Killer (NK) cells, neutrophils, and macrophages) enables these cytotoxic effector cells to bind specifically to an antigen-bearing target cell and subsequently kill the target cell with cytotoxins. Specific high-affinity IgG antibodies directed to the surface of target cells “arm” the cytotoxic cells and are absolutely required for such killing. Lysis of the target cell is extracellular, requires direct cell-to-cell contact, and does not involve complement. It is contemplated that, in addition to antibodies, other proteins comprising Fc regions, specifically Fc fusion proteins, having the capacity to bind specifically to an antigen-bearing target cell will be able to effect cell-mediated cytotoxicity. For simplicity, the cell-mediated cytotoxicity resulting from the activity of an Fc fusion protein is also referred to herein as ADCC activity.

The ability of any particular Fc variant protein to mediate lysis of the target cell by ADCC can be assayed. To assess ADCC activity an Fc variant protein of interest is added to target cells in combination with immune effector cells, which may be activated by the antigen antibody complexes resulting in cytolysis of the target cell. Cytolysis is generally detected by the release of label (e.g. radioactive substrates, fluorescent dyes or natural intracellular proteins) from the lysed cells. Useful effector cells for such assays include peripheral blood mononuclear cells (PBMC) and Natural Killer (NK) cells. Specific examples of in vitro ADCC assays are described in Wisecarver et al., 1985 79:277-282; Bruggemann et al., 1987, J Exp Med 166:1351-1361; Wilkinson et al., 2001, J Immunol Methods 258:183-191; Patel et al., 1995 J Immunol Methods 184:29-38 and herein (see Example 3). Alternatively, or additionally, ADCC activity of the Fc variant protein of interest may be assessed in vivo, e.g., in a animal model such as that disclosed in Clynes et al., 1998, PNAS USA 95:652-656.

In one embodiment, an Fc variant protein has enhanced ADCC activity relative to a comparable molecule. In a specific embodiment, an Fc variant protein has ADCC activity that is at least 2 fold, or at least 3 fold, or at least 5 fold or at least 10 fold or at least 50 fold or at least 100 fold greater than that of a comparable molecule. In another specific embodiment, an Fc variant protein has enhanced binding to the Fc receptor FcγRIIIA and has enhanced ADCC activity relative to a comparable molecule. In other embodiments, the Fc variant protein has both enhanced ADCC activity and enhanced serum half life relative to a comparable molecule.

“Complement dependent cytotoxicity” and “CDC” refer to the lysing of a target cell in the presence of complement. The complement activation pathway is initiated by the binding of the first component of the complement system (C1q) to a molecule, an antibody for example, complexed with a cognate antigen. To assess complement activation, a CDC assay, e.g. as described in Gazzano-Santoro et al., 1996, J. Immunol. Methods, 202:163, may be performed. In one embodiment, an Fc variant protein has enhanced CDC activity relative to a comparable molecule. In a specific embodiment, an Fc variant protein has CDC activity that is at least 2 fold, or at least 3 fold, or at least 5 fold or at least 10 fold or at least 50 fold or at least 100 fold greater than that of a comparable molecule. In other embodiments, the Fc variant protein has both enhanced CDC activity and enhanced serum half life relative to a comparable molecule.

In one embodiment, the present invention provides formulations, wherein the Fc region comprises a non naturally occurring amino acid residue at one or more positions selected from the group consisting of 222, 224, 234, 235, 236, 239, 240, 241, 243, 244, 245, 247, 248, 252, 254, 256, 258, 262, 263, 264, 265, 266, 267, 268, 269, 272, 274, 275, 278, 279, 280, 282, 290, 294, 295, 296, 297, 298, 299, 300, 312, 313, 318, 320, 325, 326, 327, 328, 329, 330, 332, 333, 334, 335, 339, 359, 360, 372, 377, 379, 396, 398, 400, 401, 430 and 436, as numbered by the EU index as set forth in Kabat. Optionally, the Fc region may comprise a non naturally occurring amino acid residue at additional and/or alternative positions known to one skilled in the art (see, e.g., U.S. Pat. Nos. 5,624,821; 6,277,375; 6,737,056; PCT Patent Publications WO 01/58957; WO 02/06919; WO 04/016750; WO 04/029207; WO 04/035752 and WO 05/040217).

In a specific embodiment, the present invention provides an Fc variant protein formulation, wherein the Fc region comprises at least one non naturally occurring amino acid residue selected from the group consisting of 222N, 224L, 234D, 234E, 234N, 234Q, 234T, 234H, 234Y, 234I, 234V, 234F, 235A, 235D, 235R, 235W, 235P, 235S, 235N, 235Q, 235T, 235H, 235Y, 235I, 235V, 235F, 236E, 239D, 239E, 239N, 239Q, 239F, 239T, 239H, 239Y, 240I, 240A, 240T, 240M, 241W, 241L, 241Y, 241E, 241R, 243W, 243L 243Y, 243R, 243Q, 244H, 245A, 247V, 247G, 248M, 252Y, 254T, 256E, 258D, 262I, 262A, 262T, 262E, 263I, 263A, 263T, 263M, 264L, 264I, 264W, 264T, 264R, 264F, 264M, 264Y, 264E, 265G, 265N, 265Q, 265Y, 265F, 265V, 265I, 265L, 265H, 265T, 266I, 266A, 266T, 266M, 267Q, 267L, 268D, 268N, 269H, 269Y, 269F, 269R, 296E, 272Y, 274E, 274R, 274T, 275Y, 278T, 279L, 280H, 280Q, 280Y, 282M, 290G, 290S, 290T, 290Y, 294N, 295K, 296Q, 296D, 296N, 296S, 296T, 296L, 296I, 296H, 269G, 297S, 297D, 297E, 298H, 298I, 298T, 298F, 299I, 299L, 299A, 299S, 299V, 299H, 299F, 299E, 300I, 300L, 312A, 313F, 318A, 318V, 320A, 320M, 325Q, 325L, 325I, 325D, 325E, 325A, 325T, 325V, 325H, 327G, 327W, 327N, 327L, 328S, 328M, 328D, 328E, 328N, 328Q, 328F, 328I, 328V, 328T, 328H, 328A, 329F, 329H, 329Q, 330K, 330G, 330T, 330C, 330L, 330Y, 330V, 330I, 330F, 330R, 330H, 332D, 332S, 332W, 332F, 332E, 332N, 332Q, 332T, 332H, 332Y, 332A, 335A, 335T, 335N, 335R, 335Y, 339T, 359A, 360A, 372Y, 377F, 379M, 396H, 396L, 398V, 400P, 401V, 430A, as numbered by the EU index as set forth in Kabat. Optionally, the Fc region may comprise additional and/or alternative non naturally occurring amino acid residues known to one skilled in the art (see, e.g., U.S. Pat. Nos. 5,624,821; 6,277,375; 6,737,056; PCT Patent Publications WO 01/58957; WO 02/06919; WO 04/016750; WO 04/029207; WO 04/035752 and WO 05/040217).

In one embodiment, the present invention provides an Fc variant protein formulation, wherein the Fc region comprises at least a non naturally occurring amino acid at one or more positions selected from the group consisting of 252, 254, and 256, as numbered by the EU index as set forth in Kabat. In a specific embodiment, the present invention provides an Fc variant protein formulation, wherein the Fc region comprises at least one non naturally occurring amino acid selected from the group consisting of 252Y, 254T and 256E, as numbered by the EU index as set forth in Kabat.

In another embodiment, the present invention provides an Fc variant protein formulation, wherein the Fc region comprises at least a non naturally occurring amino acid at one or more positions selected from the group consisting of 239, 330 and 332, as numbered by the EU index as set forth in Kabat. In a specific embodiment, the present invention provides an Fc variant protein formulation, wherein the Fc region comprises at least one non naturally occurring amino acid selected from the group consisting of 239D, 330L, 330Y and 332E, as numbered by the EU index as set forth in Kabat. Optionally, the Fc region may further comprise additional non naturally occurring amino acid at one or more positions selected from the group consisting of 252, 254, and 256, as numbered by the EU index as set forth in Kabat. In a specific embodiment, the present invention provides an Fc variant protein formulation, wherein the Fc region comprises at least one non naturally occurring amino acid selected from the group consisting of 239D, 330L, 330Y and 332E, as numbered by the EU index as set forth in Kabat and at least one non naturally occurring amino acid at one or more positions are selected from the group consisting of 252Y, 254T and 256E, as numbered by the EU index as set forth in Kabat.

In one embodiment, the Fc variants of the present invention may be combined with other known Fc variants such as those disclosed in Ghetie et al., 1997, Nat. Biotech. 15:637-40; Duncan et al, 1988, Nature 332:563-564; Lund et al., 1991, J. Immunol. 147:2657-2662; Lund et al, 1992, Mol Immunol 29:53-59; Alegre et al, 1994, Transplantation 57:1537-1543; Hutchins et al., 1995, Proc Natl. Acad Sci USA 92:11980-11984; Jefferis et al, 1995, Immunol Lett. 44:111-117; Lund et al., 1995, Faseb J 9:115-119; Jefferis et al, 1996, Immunol Lett 54:101-104; Lund et al, 1996, J Immunol 157:4963-4969; Armour et al., 1999, Eur J Immunol 29:2613-2624; Idusogie et al, 2000, J Immunol 164:4178-4184; Reddy et al, 2000, J Immunol 164:1925-1933; Xu et al., 2000, Cell Immunol 200:16-26; Idusogie et al, 2001, J Immunol 166:2571-2575; Shields et al., 2001, J Biol Chem 276:6591-6604; Jefferis et al, 2002, Immunol Lett 82:57-65; Presta et al., 2002, Biochem Soc Trans 30:487-490); U.S. Pat. Nos. 5,624,821; 5,885,573; 5,677,425; 6,165,745; 6,277,375; 5,869,046; 6,121,022; 5,624,821; 5,648,260; 6,528,624; 6,194,551; 6,737,056; 6,821,505; 6,277,375; U.S. Patent Publication Nos. 2004/0002587 and PCT Publications WO 94/29351; WO 99/58572; WO 00/42072; WO 02/060919; WO 04/029207; WO 04/099249; WO 04/063351. Also encompassed by the present invention are Fc regions which comprise deletions, additions and/or modifications. Still other modifications/substitutions/additions/deletions of the Fc domain will be readily apparent to one skilled in the art.

It is specifically contemplated that conservative amino acid substitutions may be made for any of the substitutions described supra. It is well known in the art that “conservative amino acid substitution” refers to amino acid substitutions that substitute functionally-equivalent amino acids. Conservative amino acid changes result in silent changes in the amino acid sequence of the resulting peptide. For example, one or more amino acids of a similar polarity act as functional equivalents and result in a silent alteration within the amino acid sequence of the peptide. Substitutions that are charge neutral and which replace a residue with a smaller residue may also be considered “conservative substitutions” even if the residues are in different groups (e.g., replacement of phenylalanine with the smaller isoleucine). Families of amino acid residues having similar side chains have been defined in the art. Several non-limiting families of conservative amino acid substitutions are shown in Table 1.

TABLE 1
Families of Conservative Amino Acid Substitutions

| Family | Amino Acids |
|---|---|
| non-polar | Trp, Phe, Met, Leu, Ile, Val, |
| | Ala, Pro |
| uncharged polar | Gly, Ser, Thr, Asn, Gln, Tyr, |
| | Cys |
| acidic/negatively charged | Asp, Glu |
| basic/positively charged | Arg, Lys, His |
| Beta-branched | Thr, Val, Ile |
| residues that influence chain orientation | Gly, Pro |
| aromatic | Trp, Tyr, Phe, His |

The term “conservative amino acid substitution” also refers to the use of amino acid analogs or variants. Guidance concerning how to make phenotypically silent amino acid substitutions is provided in Bowie et al., “Deciphering the Message in Protein Sequences: Tolerance to Amino Acid Substitutions,” (1990, Science 247:1306-1310).

Methods for generating non naturally occurring Fc regions are known in the art. For example, amino acid substitutions and/or deletions can be generated by mutagenesis methods, including, but not limited to, site-directed mutagenesis (Kunkel, Proc. Natl. Acad. Sci. USA 82:488-492 (1985)), PCR mutagenesis (Higuchi, in “PCR Protocols: A Guide to Methods and Applications”, Academic Press, San Diego, pp. 177-183 (1990)), and cassette mutagenesis (Wells et al., Gene 34:315-323 (1985)). Preferably, site-directed mutagenesis is performed by the overlap-extension PCR method, which is disclosed in the Examples (Higuchi, in “PCR Technology: Principles and Applications for DNA Amplification”, Stockton Press, New York, pp. 61-70 (1989)). Alternatively, the technique of overlap-extension PCR (Higuchi, ibid.) can be used to introduce any desired mutation(s) into a target sequence (the starting DNA). For example, the first round of PCR in the overlap-extension method involves amplifying the target sequence with an outside primer (primer 1) and an internal mutagenesis primer (primer 3), and separately with a second outside primer (primer 4) and an internal primer (primer 2), yielding two PCR segments (segments A and B). The internal mutagenesis primer (primer 3) is designed to contain mismatches to the target sequence specifying the desired mutation(s). In the second round of PCR, the products of the first round of PCR (segments A and B) are amplified by PCR using the two outside primers (primers 1 and 4). The resulting full-length PCR segment (segment C) is digested with restriction enzymes and the resulting restriction fragment is cloned into an appropriate vector. As the first step of mutagenesis, the starting DNA (e.g., encoding an Fc fusion protein, an antibody or simply an Fc region), is operably cloned into a mutagenesis vector. The primers are designed to reflect the desired amino acid substitution. Other methods useful for the generation of variant Fc regions are known in the art (see, e.g., U.S. Pat. Nos. 5,624,821; 5,885,573; 5,677,425; 6,165,745; 6,277,375; 5,869,046; 6,121,022; 5,624,821; 5,648,260; 6,528,624; 6,194,551; 6,737,056; 6,821,505; 6,277,375; U.S. Patent Publication Nos. 2004/0002587 and PCT Publications WO 94/29351; WO 99/58572; WO 00/42072; WO 02/060919; WO 04/029207; WO 04/099249; WO 04/063351).

In some embodiments, an Fc variant protein comprises one or more engineered glycoforms, i.e., a carbohydrate composition that is covalently attached to the molecule comprising an Fc region. Engineered glycoforms may be useful for a variety of purposes, including but not limited to enhancing or reducing effector function. Engineered glycoforms may be generated by any method known to one skilled in the art, for example by using engineered or variant expression strains, by co-expression with one or more enzymes, for example DI N-acetylglucosaminyltransferase III (GnTI11), by expressing a molecule comprising an Fc region in various organisms or cell lines from various organisms, or by modifying carbohydrate(s) after the molecule comprising Fc region has been expressed. Methods for generating engineered glycoforms are known in the art, and include but are not limited to those described in Umana et al, 1999, Nat. Biotechnol 17:176-180; Davies et al., 20017 Biotechnol Bioeng 74:288-294; Shields et al, 2002, J Biol Chem 277:26733-26740; Shinkawa et al., 2003, J Biol Chem 278:3466-3473) U.S. Pat. No. 6,602,684; U.S. Ser. No. 10/277,370; U.S. Ser. No. 10/113,929; PCT WO 00/61739A1; PCT WO 01/292246A1; PCT WO 02/311140A1; PCT WO 02/30954A1; Potillegent™ technology (Biowa, Inc. Princeton, N.J.); GlycoMAb™ glycosylation engineering technology (GLYCART biotechnology AG, Zurich, Switzerland). See, e.g., WO 00061739; EA01229125; US 20030115614; Okazaki et al., 2004, JMB, 336: 1239-49. Additional methods are described below in the section entitled “Antibodies”.

### 6.4 Fc Variant Proteins

As described above, an Fc variant protein is a protein comprising a variant Fc region or fragment thereof including, but are not limited to, antibodies and Fc fusion proteins. An Fc fusion combines an Fc region or fragment thereof, with a fusion partner, which in general can be any protein, polypeptide, peptide, including, but not limited to, the target-binding region of a receptor, an adhesion molecule, a ligand, an enzyme, or some other protein or protein domain. Also encompassed by the invention are Fc fusion proteins comprising an Fc region, or fragment thereof, fused to a small molecule. The role of the non-Fc part of an Fc fusion is to mediate target binding, and thus it is functionally analogous to the variable regions of an antibody. Accordingly, in one embodiment, an Fc variant protein is an antibody. In another embodiment, an Fc variant protein is an Fc fusion protein.

An variant Fc protein may be produced “de novo” by combining a protein or fragment thereof (e.g., a variable domain that immunospecifically binds an antigen of interest or the extracellular domain of a receptor of interest) with a variant Fc region or fragment thereof. Alternatively, may be produced by modifying an Fc region-containing protein (e.g., and antibody that binds an antigen of interest or an Fc fusion protein) by introducing one or more non naturally occurring residues into the Fc region.

### 6.4.1 Antibodies

Antibodies are immunological proteins that bind a specific antigen which comprise a variable region and may further comprise one or more constant regions. The constant regions show less sequence diversity, and are responsible for binding a number of natural proteins to elicit important biochemical events. The variable region of an antibody contains the antigen binding determinants of the molecule, and thus determines the specificity of an antibody for its target antigen. The variable region is so named because it is the most distinct in sequence from other antibodies within the same class. The majority of sequence variability occurs in the complementarity determining regions (CDRs). There are 6 CDRs total, three each per heavy and light chain, designated VH CDR1, VH CDR2, VH CDR3, VL CDR1, VL CDR2, and VL CDR3. The variable region outside of the CDRs is referred to as the framework (FR) region. Although not as diverse as the CDRs, sequence variability does occur in the FR region between different antibodies. It will be understood that the complementarity determining regions (CDRs) residue numbers referred to herein are those of Kabat et al. (1991, NIH Publication 91-3242, National Technical Information Service, Springfield, Va.). Specifically, residues 24-34 (CDR1), 50-56 (CDR2) and 89-97 (CDR3) in the light chain variable domain and 31-35 (CDR1), 50-65 (CDR2) and 95-102 (CDR3) in the heavy chain variable domain. Note that CDRs vary considerably from antibody to antibody (and by definition will not exhibit homology with the Kabat consensus sequences). Maximal alignment of framework residues frequently requires the insertion of “spacer” residues in the numbering system, to be used for the Fv region. It will be understood that the CDRs referred to herein are those of Kabat et al. supra. In addition, the identity of certain individual residues at any given Kabat site number may vary from antibody chain to antibody chain due to interspecies or allelic divergence.

As used herein, the terms “antibody” and “antibodies” refer to a protein consisting of one or more polypeptides substantially encoded by all or part of the recognized immunoglobulin genes and includes, but is not limited to, monoclonal antibodies, multispecific antibodies, human antibodies, humanized antibodies, camelised antibodies, chimeric antibodies, single-chain Fvs (scFv), disulfide-linked Fvs (sdFv), Fab fragments, F (ab′) fragments, and anti-idiotypic (anti-Id) antibodies (including, e.g., anti-Id antibodies to antibodies of the invention), and epitope-binding fragments of any of the above fused to an Fc region or fragment thereof. Antibodies used in the methods of the present invention include immunoglobulin molecules and immunologically active portions of immunoglobulin molecules, i.e., molecules that contain an antigen binding site. In specific embodiments, these fragments are fused to an Fc region or fragment thereof which may or may not be a variant Fc region. As outlined herein, the terms “antibody” and “antibodies” specifically include antibodies comprising a variant Fc region as described herein, full length antibodies and Fc-fusions comprising variant Fc regions, or fragments thereof, described herein fused to an immunologically active fragment of an immunoglobulin or to other proteins as described herein. Such Fc variant-fusions include but are not limited to, scFv-Fc fusions, variable region (e.g., VL and VH)—Fc fusions, scFv-scFv-Fc fusions. The immunoglobulin molecules of the invention can be of any type (e.g., IgG, IgE, IgM, IgD, IgA and IgY), class (e.g., IgG1, IgG2, IgG3, IgG4, IgA1 and IgA2) or subclass of immunoglobulin molecule.

Antibodies or antibody fragments may be from any animal origin including birds and mammals (e.g., human, murine, donkey, sheep, rabbit, goat, guinea pig, camel, horse, or chicken). In one embodiment, the antibodies are human or humanized monoclonal antibodies. As used herein, “human” antibodies include antibodies having the amino acid sequence of a human immunoglobulin and include antibodies isolated from human immunoglobulin libraries or from mice that express antibodies from human genes.

Antibodies like all polypeptides have an Isoelectric Point (pI), which is generally defined as the pH at which a polypeptide carries no net charge. It is known in the art that protein solubility is typically lowest when the pH of the solution is equal to the isoelectric point (pI) of the protein. It is possible to optimize solubility by altering the number and location of ionizable residues in the antibody to adjust the pI. For example the pI of a polypeptide can be manipulated by making the appropriate amino acid substitutions (e.g., by substituting a charged amino acid such as a lysine, for an uncharged residue such as alanine). Without wishing to be bound by any particular theory, amino acid substitutions of an antibody that result in changes of the pI of said antibody may improve solubility and/or the stability of the antibody. One skilled in the art would understand which amino acid substitutions would be most appropriate for a particular antibody to achieve a desired pI. The pI of a protein may be determined by a variety of methods including but not limited to, isoelectric focusing and various computer algorithms (see for example Bjellqvist et al., 1993, Electrophoresis 14:1023-1031). In one embodiment, the pI of an antibody utilized in accordance with the invention is higher then about 6.5, about 7.0, about 7.5, about 8.0, about 8.5, or about 9.0. In a specific embodiment, substitutions resulting in alterations in the pI of the antibody will not significantly diminish its binding affinity for its antigen. In another embodiment, the pI of an antibody utilized in accordance with the invention is higher than 6.5, 7.0, 7.5, 8.0, 8.5, or 9.0. It is specifically contemplated that the substitution(s) of the Fc region that result in altered binding to one or more Fc ligand (described supra) may also result in a change in the pI. In another embodiment, substitution(s) of the Fc region are specifically chosen to effect both the desired alteration in FcγR binding and any desired change in pI. As used herein the pI value is defined as the pI of the predominant charge form. The pI of a protein may be determined by a variety of methods including but not limited to, isoelectric focusing and various computer algorithms (see, e.g., Bjellqvist et al., 1993, Electrophoresis 14:1023).

The Tm of the Fab domain of an antibody can be a good indicator of the thermal stability of an antibody and may further provide an indication of the shelf-life. A lower Tm indicates more aggregation/less stability, whereas a higher Tm indicates less aggregation/more stability. Thus, antibodies having higher Tm are preferable. In one embodiment, the Fab domain of an antibody utilized in accordance with the invention has a Tm value higher than at least 50° C., 55° C., 60° C., 65° C., 70° C., 75° C., 80° C., 85° C., 90° C., 95° C., 100° C., 105° C., 110° C., 115° C. or 120° C. Thermal melting temperatures (Tm) of a protein domain (e.g., a Fab domain) can be measured using any standard method known in the art, for example, by differential scanning calorimetry (see, e.g., Vermeer et al., 2000, Biophys. J. 78:394-404; Vermeer et al., 2000, Biophys. J. 79: 2150-2154). In addition, the Tm of an antibody formulated in different buffer may be examined to determine the impact of the formulation of antibody stability.

Antibodies or antibody fragments used in accordance with the present invention may be monospecific, bispecific, trispecific or of greater multispecificity. Multispecific antibodies may immunospecifically bind to different epitopes of desired target molecule or may immunospecifically bind to both the target molecule as well as a heterologous epitope, such as a heterologous polypeptide or solid support material. See, e.g., International Publication Nos. WO 93/17715, WO 92/08802, WO 91/00360, and WO 92/05793; Tutt, et al., 1991, J. Immunol. 147:60-69; U.S. Pat. Nos. 4,474,893, 4,714,681, 4,925,648, 5,573,920, and 5,601,819; and Kostelny et al., 1992, J. Immunol. 148:1547-1553.

Multispecific antibodies have binding specificities for at least two different antigens. While such molecules normally will only bind two antigens (i.e. bispecific antibodies, BsAbs), antibodies with additional specificities such as trispecific antibodies are encompassed by the instant invention. Examples of BsAbs include without limitation those with one arm directed against a first antigen and the other arm directed against a second antigen. Methods for making bispecific antibodies are known in the art. Traditional production of full-length bispecific antibodies is based on the coexpression of two immunoglobulin heavy chain-light chain pairs, where the two chains have different specificities (Millstein et al., 1983, Nature, 305:537-539). Because of the random assortment of immunoglobulin heavy and light chains, these hybridomas (quadromas) produce a potential mixture of different antibody molecules, of which only one has the correct bispecific structure. Purification of the correct molecule, which is usually done by affinity chromatography steps, is rather cumbersome, and the product yields are low. Similar procedures are disclosed in WO 93/08829, and in Traunecker et al., 1991, EMBO J., 10:3655-3659. A more directed approach is the generation of a Di-diabody a tetravalent bispecific antibody. Methods for producing a Di-diabody are known in the art (see e.g., Lu et al., 2003, J Immunol Methods 279:219-32; Marvin et al., 2005, Acta Pharmacolical Sinica 26:649).

According to a different approach, antibody variable domains with the desired binding specificities (antibody-antigen combining sites) are fused to immunoglobulin constant domain sequences. The fusion preferably is with an immunoglobulin heavy chain constant domain, comprising at least part of the hinge, CH2, and CH3 regions. It is preferred to have the first heavy-chain constant region (CH1) containing the site necessary for light chain binding, present in at least one of the fusions. DNAs encoding the immunoglobulin heavy chain fusions and, if desired, the immunoglobulin light chain, are inserted into separate expression vectors, and are co-transfected into a suitable host organism. This provides for great flexibility in adjusting the mutual proportions of the three polypeptide fragments in embodiments when unequal ratios of the three polypeptide chains used in the construction provide the optimum yields. It is, however, possible to insert the coding sequences for two or all three polypeptide chains in one expression vector when, the expression of at least two polypeptide chains in equal ratios results in high yields or when the ratios are of no particular significance.

In one embodiment of this approach, the bispecific antibodies are composed of a hybrid immunoglobulin heavy chain with a first binding specificity in one arm, and a hybrid immunoglobulin heavy chain-light chain pair (providing a second binding specificity) in the other arm. It was found that this asymmetric structure facilitates the separation of the desired bispecific compound from unwanted immunoglobulin chain combinations, as the presence of an immunoglobulin light chain in only one half of the bispecific molecule provides for a facile way of separation. This approach is disclosed in WO 94/04690. For further details of generating bispecific antibodies see, for example, Suresh et al., 1986, Methods in Enzymology, 121:210. According to another approach described in WO96/27011, a pair of antibody molecules can be engineered to maximize the percentage of heterodimers which are recovered from recombinant cell culture. The preferred interface comprises at least a part of the CH3 domain of an antibody constant domain. In this method, one or more small amino acid side chains from the interface of the first antibody molecule are replaced with larger side chains (e.g. tyrosine or tryptophan). Compensatory “cavities” of identical or similar size to the large side chain(s) are created on the interface of the second antibody molecule by replacing large amino acid side chains with smaller ones (e.g. alanine or threonine). This provides a mechanism for increasing the yield of the heterodimer over other unwanted end-products such as homodimers.

Bispecific antibodies include cross-linked or “heteroconjugate” antibodies. For example, one of the antibodies in the heteroconjugate can be coupled to avidin, the other to biotin. Such antibodies have, for example, been proposed to target immune system cells to unwanted cells (U.S. Pat. No. 4,676,980), and for treatment of HIV infection (WO 91/00360, WO 92/200373, and EP 03089). Heteroconjugate antibodies may be made using any convenient cross-linking methods. Suitable cross-linking agents are well known in the art, and are disclosed in U.S. Pat. No. 4,676,980, along with a number of cross-linking techniques. Antibodies with more than two valencies are contemplated. For example, trispecific antibodies can be prepared. See, e.g., Tutt et al. J. Immunol. 147: 60 (1991).

Other antibodies specifically contemplated are “oligoclonal” antibodies. As used herein, the term “oligoclonal” antibodies” refers to a predetermined mixture of distinct monoclonal antibodies. See, e.g., PCT publication WO 95/20401; U.S. Pat. Nos. 5,789,208 and 6,335,163. In one embodiment, oligoclonal antibodies consist of a predetermined mixture of antibodies against one or more epitopes are generated in a single cell. In another embodiment, oligoclonal antibodies comprise a plurality of heavy chains, having non naturally occurring amino acids, capable of pairing with a common light chain to generate antibodies with multiple specificities (e.g., PCT publication WO 04/009618). Oligoclonal antibodies are particularly useful when it is desired to target multiple epitopes on a single target molecule. Those skilled in the art will know or can determine what type of antibody or mixture of antibodies is applicable for an intended purpose and desired need.

The present invention may also be practiced with single domain antibodies, including camelized single domain antibodies (see e.g., Muyldermans et al., 2001, Trends Biochem. Sci. 26:230; Nuttall et al., 2000, Cur. Pharm. Biotech. 1:253; Reichmann and Muyldermans, 1999, J. Immunol. Meth. 231:25; International Publication Nos. WO 94/04678 and WO 94/25591; U.S. Pat. No. 6,005,079).

Antibodies which may be utilized in accordance with the invention also encompasses those that have half-lives (e.g., serum half-lives) in a mammal, (e.g., a human), of greater than 5 days, greater than 10 days, greater than 15 days, greater than 20 days, greater than 25 days, greater than 30 days, greater than 35 days, greater than 40 days, greater than 45 days, greater than 2 months, greater than 3 months, greater than 4 months, or greater than 5 months. The increased half-lives of an antibodies in a mammal, (e.g., a human), results in a higher serum titer of said antibodies or antibody fragments in the mammal, and thus, reduces the frequency of the administration of said antibodies or antibody fragments and/or reduces the concentration of said antibodies or antibody fragments to be administered. Antibodies having increased in vivo half-lives can be generated by techniques known to those of skill in the art. For example, as described above antibodies with increased in vivo half-lives can be generated by modifying (e.g., substituting, deleting or adding) amino acid residues identified as involved in the interaction between the Fc domain and the FcRn receptor (see, e.g., International Publication Nos. WO 97/34631; WO 04/029207; U.S. Pat. No. 6,737,056 and U.S. Patent Publication No. 2003/0190311).

In still another embodiment, the glycosylation of antibodies utilized in accordance with the invention is modified. For example, an aglycoslated antibody can be made (i.e., the antibody lacks glycosylation). Glycosylation can be altered to, for example, increase the affinity of the antibody for a target antigen. Such carbohydrate modifications can be accomplished by, for example, altering one or more sites of glycosylation within the antibody sequence. For example, one or more amino acid substitutions can be made that result in elimination of one or more variable region framework glycosylation sites to thereby eliminate glycosylation at that site. Such aglycosylation may increase the affinity of the antibody for antigen. Such an approach is described in further detail in U.S. Pat. Nos. 5,714,350 and 6,350,861. Alternatively, one or more amino acid substitutions can be made that result in elimination of a glycosylation site present in the Fc region (e.g., Asparagine 297 of IgG). Furthermore, a glycosylated antibodies may be produced in bacterial cells which lack the necessary glycosylation machinery.

Additionally or alternatively, an antibody can be made that has an altered type of glycosylation, such as a hypofucosylated antibody having reduced amounts of fucosyl residues or an antibody having increased bisecting GlcNAc structures. Such altered glycosylation patterns have been demonstrated to increase the ADCC ability of antibodies. Such carbohydrate modifications can be accomplished by, for example, expressing the antibody in a host cell with altered glycosylation machinery. Cells with altered glycosylation machinery have been described in the art and can be used as host cells in which to express recombinant antibodies of the invention to thereby produce an antibody with altered glycosylation. See, for example, Shields, R. L. et al. (2002) J. Biol. Chem. 277:26733-26740; Umana et al. (1999) Nat. Biotech. 17:176-1, as well as, European Patent No: EP 1,176,195; PCT Publications WO 03/035835; WO 99/54342.

Also encompassed by the present invention are “antibody-like” and “antibody-domain fusion” proteins. An antibody-like molecule is any molecule that has been generated with a desired binding property, see, e.g., PCT Publication Nos. WO 04/044011; WO 04/058821; WO 04/003019 and WO 03/002609. Antibody-domain fusion proteins may incorporate one or more antibody domains such as the variable domain with an Fc region. For example, the heterologous polypeptides may be fused or conjugated to a Fab fragment, Fd fragment, Fv fragment, F(ab)₂ fragment, a VH domain, a VL domain, a VH CDR, a VL CDR, or fragment thereof which is then fused to an Fc region, such as a variant Fc region and formulated according to the present invention. A large number of antibody-domain molecules are known in the art including, but not limited to, diabodies (dsFv)₂ (Bera et al., 1998, J. Mol. Biol. 281:475-83); minibodies (homodimers of scFv-CH3 fusion proteins)(Pessi et al., 1993, Nature 362:367-9), tetravalent di-diabody (Lu et al., 2003 J. Immunol. Methods 279:219-32), tetravalent bi-specific antibodies called Bs(scFv)₄-IgG (Zuo et al., 2000, Protein Eng. 13:361-367). These molecules may be fused to a variant Fc region or may be modified to comprise non naturally occurring amino acid residues in existing Fc regions. Methods for fusing or conjugating polypeptides to antibody portions are well known in the art. See, e.g., U.S. Pat. Nos. 5,336,603, 5,622,929, 5,359,046, 5,349,053, 5,447,851, and 5,112,946; European Patent Nos. EP 307,434 and EP 367,166; PCT Publication Nos. WO 96/04388 and WO 91/06570; Ashkenazi et al., 1991, Proc. Natl. Acad. Sci. USA 88: 10535-10539; Zheng et al., 1995, J. Immunol. 154:5590-5600; and Vil et al., 1992, Proc. Natl. Acad. Sci. USA 89:11337-11341. Other molecules specifically contemplated are small, engineered protein domains such as, for example, immuno-domains and/or monomer domains (see for example, U.S. Patent Publication Nos. 2003082630 and 2003157561). Immuno-domains contain at least one complementarity determining region (CDR) of an antibody while monomer domains are based upon known naturally-occurring, non-antibody domain families, specifically protein extracellular domains, which contain conserved scaffold and variable binding sites, an example is the LDL receptor extracellular domain, a domain which is involved in ligand binding. Such protein domains can correctly fold independently or with limited assistance from, for example, a chaperonin or the presence of a metal ion. This ability avoids mis-folding of the domain when it is inserted into a new protein environment, thereby preserving the protein domain's binding affinity for a particular target. The variable binding sites of the protein domains are randomized using various diversity generation methods such as, for example, random mutagenesis, site-specific mutagenesis, as well as by directed evolution methods, such as, for example, recursive error-prone PCR, recursive recombination and the like. For details of various diversity generation methods see U.S. Pat. Nos. 5,811,238; 5,830,721; 5,834,252; PCT Publication Nos. WO 95/22625; WO 96/33207; WO 97/20078; WO 97/35966; WO 99/41368; WO 99/23107; WO 00/00632; WO 00/42561; and WO 01/23401. The mutagenized protein domains are then expressed using a display system such as, for example, phage display, which can generate a library of at least 10¹⁰ variants and facilitate isolation of those protein domains with improved affinity and potency for an intended target by subsequent panning and screening. Such methods are described in PCT publication Nos. WO 91/17271; WO 91/18980; WO 91/19818; WO 93/08278. Examples of additional display systems are described in U.S. Pat. Nos. 6,281,344; 6,194,550; 6,207,446; 6,214,553 and 6,258,558. Utilizing these methods a high diversity of engineered protein domains having sub-nM binding affinity (Kd) and blocking function (IC50) can be rapidly generated. Once identified two to ten such engineered protein domains can be linked together, using natural protein linkers of about 4-15 amino acids in length, to form a binding protein. The individual domains can target a single type of protein or several, depending upon the use/disease indication. The engineered protein domains can then be linked to a variant Fc region to generate an Fc variant protein.

### 6.4.2 Fc Fusion Proteins

As described above the formulations of the invention encompasses formulations of Fc fusion proteins. Fc fusion proteins combine the Fc region or fragment thereof of an immunoglobulin with a fusion partner which in general can be an protein, including, but not limited to, an antigen binding portion of an antibody, a ligand, an enzyme, the ligand portion of a receptor, an adhesion protein, or some other protein or domain. See, e.g., Chamow et al., 1996, Trends Biotechnol 14:52-60; Ashkenazi et al., 1997, Curr Opin Immunol 9:195-200; Heidaran et al., 1995, FASEB J. 9:140-5. Methods for fusing or conjugating polypeptides to Fc regions are well known in the art. See, e.g., U.S. Pat. Nos. 5,336,603; 5,349,053; 5,447,851; 5,783,181; European Patent No. EP 367,166; International publication Nos. WO 91/06570; Ashkenazi et al., 1991, Proc. Natl. Acad. Sci. USA 88: 10535-10539; Zheng et al., 1995, J. Immunol. 154:5590-5600; and Vie et al., 1992, Proc. Natl. Acad. Sci. USA 89:11337-11341. It is contemplated that an Fc fusion protein comprising a variant Fc region may be formulated according to the present invention to improve stability (e.g., reduce aggregation). An Fc fusion protein comprising a variant Fc region may be generated, for example, by fusing or conjugating a heterologous polypeptide to an Fc region or fragment thereof, which comprises one or more non naturally occurring amino acid residues (i.e., a variant Fc region). Alternatively, the Fc region of an Fc fusion protein may be modified by introducing one or more non naturally occurring residues into the Fc region to generate a variant Fc region.

In one embodiment, an Fc fusion protein that binds to a molecule (i.e., target) comprises a fusion partner fused to a variant Fc region including, but not limited to, those disclosed herein. In accordance with these embodiments, the fusion partner binds to a molecule (i.e., target). Fusion partners that may be fused to a variant Fc region include, but are not limited to, peptides, polypeptides, proteins, small molecules, mimetic agents, synthetic drugs, inorganic molecules, and organic molecules. In one embodiment, a fusion partner is a polypeptide comprising at least 5, at least 10, at least 20, at least 30, at least 40, at least 50, at least 60, at least 70, at least 80, at least 90 or at least 100 contiguous amino acid residues, and is heterologous to the amino acid sequence of the variant Fc region.

### 6.4.3 Antigens, Fusion Partners and Antibodies

Virtually any molecule may be targeted by and/or incorporated into an Fc variant protein (e.g., antibodies, Fc fusion proteins) including, but not limited to, the following list of proteins, as well as subunits, domains, motifs and epitopes belonging to the following list of proteins: renin; a growth hormone, including human growth hormone and bovine growth hormone; growth hormone releasing factor; parathyroid hormone; thyroid stimulating hormone; lipoproteins; alpha-1-antitrypsin; insulin A-chain; insulin B-chain; proinsulin; follicle stimulating hormone; calcitonin; luteinizing hormone; glucagon; clotting factors such as factor VII, factor VIIIC, factor IX, tissue factor (TF), and von Willebrands factor; anti-clotting factors such as Protein C; atrial natriuretic factor; lung surfactant; a plasminogen activator, such as urokinase or human urine or tissue-type plasminogen activator (t-PA); bombesin; thrombin; hemopoietic growth factor; tumor necrosis factor (TNF) proteins such as TNF-alpha, TNF-beta, TNFbeta2, TNFα, TNFalphabeta, 4-1BBL as well as members of the TNF superfamily members such as, TNF-like weak inducer of apoptosis (TWEAK), and LIGHT, B lymphocyte stimulator (BlyS); members of the TNF receptor superfamily including TNF-RI, TNF-RII, TRAIL receptor-1, CD137, Transmembrane activator and CAML interactor (TACI) and OX40L; Fas ligand (FasL); enkephalinase; RANTES (regulated on activation normally T-cell expressed and secreted); human macrophage inflammatory protein (MIP-1-alpha); a serum albumin such as human serum albumin; Muellerian-inhibiting substance; relaxin A-chain; relaxin B-chain; prorelaxin; mouse gonadotropin-associated peptide; a microbial protein, such as beta-lactamase; DNase; IgE; a cytotoxic T-lymphocyte associated antigen (CTLA), such as CTLA-4; inhibin; activin; vascular endothelial growth factor (VEGF); receptors for hormones or growth factors such as, for example, EGFR (ErbB-1), VGFR, CTGF (connective tissue growth factor); interferons such as alpha interferon (α-IFN), beta interferon (β-IFN) and gamma interferon (γ-IFN); interferon alpha receptor (IFNAR) subunits 1 and/or 2 and other receptors such as, A1, Adenosine Receptor, Lymphotoxin Beta Receptor, BAFF-R, endothelin receptor; protein A or D; rheumatoid factors; a neurotrophic factor such as bone-derived neurotrophic factor (BDNF), neurotrophin-3, -4, -5, or -6 (NT-3, NT-4, NT-5, or NT-6), or a nerve growth factor; platelet-derived growth factor (PDGF); fibroblast growth factor such as αFGF and βFGF; epidermal growth factor (EGF); transforming growth factor (TGF) such as TGF-alpha and TGF-beta, including TGF-1, TGF-2, TGF-3, TGF-4, or TGF-5; insulin-like growth factor-I and -II (IGF-I and IGF-II); des (1-3)-IGF-I (brain IGF-I), insulin-like growth factor binding proteins, keratinocyte growth factor; growth factor receptors such as, FGFR-3, IGFR, PDGFRα; CD proteins such as CD2, CD3, CD3E, CD4, CD 8, CD11, CD11a, CD14, CD16, CD18, CD19, CD20, CD22, CD23, CD25, CD27, CD27L, CD28, CD29, CD30, CD30L, CD32, CD33 (p67 protein), CD34, CD38, CD40, CD40L, CD44, CD45, CD52, CD54, CD55, CD56, CD63, CD64, CD80; CD137 and CD147; IL-2R/IL-15R Beta Subunit (CD 122); erythropoietin; osteoinductive factors; immunotoxins; a bone morphogenetic protein (BMP); an interferon such as interferon-alpha, -beta, and -gamma; colony stimulating factors (CSFs), such as M-CSF, GM-CSF, and G-CSF; interleukins (ILs), e.g., IL-1 to IL-13 and IL-15, IL-18, IL-23; EPO; superoxide dismutase; T-cell receptors alpha/beta; surface membrane proteins; decay accelerating factor; viral antigen such as, for example, a portion of the AIDS envelope, e.g., gp120; transport proteins; homing receptors; addressins; regulatory proteins; chemokine family members such as the eotaxins, the MIPs, MCP-1, RANTES; cell adhesion molecules such as selectins (L-selectin, P-selectin, E-selectin) LFA-1, LFA-3, Mac 1, p150.95, VLA-1, VLA-4, ICAM-1, ICAM-3, EpCAM and VCAM, a4/p7 integrin, and Xv/p3 integrin, integrin alpha subunits such as CD49a, CD49b, CD49c, CD49d, CD49e, CD49f, alpha7, alpha8, alpha9, alphaD, CD11a, CD11b, CD51, CD11c, CD41, alphaIIb, alphaIELb; integrin beta subunits such as, CD29, CD 18, CD61, CD104, beta5, beta6, beta7 and beta8; Integrin subunit combinations including but not limited to, αVβ3, αVβ5 and α4β7; cellular ligands such as, TNF-related apoptosis-inducing ligand (TRAIL), A proliferation-inducing ligand (APRIL), B Cell Activating Factor (BAFF), a member of an apoptosis pathway; IgE; blood group antigens; flk2/flt3 receptor; obesity (OB) receptor; mpl receptor; CTLA-4; protein C; an Eph receptor such as EphA2, EphA4, EphB2, etc.; immune system markers, receptors and ligands such as CTLA-4, T cell receptor, B7-1, B7-2, IgE, Human Leukocyte Antigen (HLA) such as HLA-DR, CBL; complement proteins such as complement receptor CR1, C1Rq and other complement factors such as C3, and C5; blood factors including tissue factor, factor VII; a glycoprotein receptor such as GpIba, GPIIb/IIIa and CD200; and fragments of any of the above-listed polypeptides.

Also contemplated are cancer related proteins including, but not limited to, ALK receptor (pleiotrophin receptor), pleiotrophin; KS ¼ pan-carcinoma antigen; ovarian carcinoma antigen (CA125); prostatic acid phosphate; prostate specific antigen (PSA); prostate specific membrane antigen (PSMA); melanoma-associated antigen p97; melanoma antigen gp75; high molecular weight melanoma antigen (HMW-MAA); prostate specific membrane antigen; carcinoembryonic antigen (CEA); carcinoembryonic antigen-related cell adhesion molecule (CEACAM1); cytokeratin tumor-associated antigen; human milk fat globule (HMFG) antigen; CanAg antigen; tumor-associated antigen expressing Lewis Y related carbohydrate; colorectal tumor-associated antigens such as: CEA, tumor-associated glycoprotein-72 (TAG-72), CO17-1A, GICA 19-9, CTA-1 and LEA; Burkitt's lymphoma antigen-38.13; CD19; human B-lymphoma antigen-CD20; CD22; CD33; melanoma specific antigens such as ganglioside GD2, ganglioside GD3, ganglioside GM2 and ganglioside GM3; tumor-specific transplantation type cell-surface antigen (TSTA); virally-induced tumor antigens including T-antigen, DNA tumor viruses and Envelope antigens of RNA tumor viruses; oncofetal antigen-alpha-fetoprotein such as CEA of colon, 5T4 oncofetal trophoblast glycoprotein and bladder tumor oncofetal antigen; differentiation antigen such as human lung carcinoma antigens L6 and L20; antigens of fibrosarcoma; human leukemia T cell antigen-Gp37; neoglycoprotein; sphingolipids; breast cancer antigens such as EGFR (Epidermal growth factor receptor); NY-BR-16; NY-BR-16 and HER2 antigen (p185^{HER2}); Her2/neu (ErbB-2), Her3 (ErbB-3), Her4 (ErbB-4), polymorphic epithelial mucin (PEM) antigen; epithelial membrane antigen (EMA); Melanoma-associated antigen MUC18; MUC1; malignant human lymphocyte antigen-APO-1; differentiation antigen such as I antigen found in fetal erythrocytes; primary endoderm I antigen found in adult erythrocytes; preimplantation embryos; I(Ma) found in gastric adenocarcinomas; M18, M39 found in breast epithelium; SSEA-1 found in myeloid cells; VEP8; VEP9; Myl; VIM-D5; D₁56-22 found in colorectal cancer; TRA-1-85 (blood group H); SCP-1 found in testis and ovarian cancer; C14 found in colonic adenocarcinoma; F3 found in lung adenocarcinoma; AH6 found in gastric cancer; Y hapten; Le^{y} found in embryonal carcinoma cells; Colonocyte differentiation antigen found in colorectal tumors, Carbonic anhydrase IX found in renal cell carcinoma, FAPa in the stroma around numerous tumor types, Folate binding protein found in ovarian tumors, PD1; death receptor proteins, DR5; TL5 (blood group A); EGF receptor found in A431 cells; E₁ series (blood group B) found in pancreatic cancer; FC10.2 found in embryonal carcinoma cells; gastric adenocarcinoma antigen; CO-514 (blood group Le^{a}) found in Adenocarcinoma; NS-10 found in adenocarcinomas; CO-43 (blood group Le^{b}); G49 found in EGF receptor of A431 cells; MH2 (blood group ALe^{b}/Le^{y}) found in colonic adenocarcinoma; 19.9 found in colon cancer; gastric cancer mucins; T₅A₇ found in myeloid cells; R₂₄ found in melanoma; 4.2, G_{D3}, D1.1, OFA-1, GM2, OFA-2, G_{D2}, and M1:22:25:8 found in embryonal carcinoma cells and SSEA-3 and SSEA-4 found in 4 to 8-cell stage embryos; Cutaneous T cell Lymphoma antigen; MART-1 antigen; Sialy Tn (STn) antigen; Anaplastic lymphoma kinase (ALK) found in large cell lymphoma; Colon cancer antigen NY-CO-45; Lung cancer antigen NY-LU-12 variant A; Adenocarcinoma antigen ART1; Paraneoplastic associated brain-testis-cancer antigen (onconeuronal antigen MA2; paraneoplastic neuronal antigen); Neuro-oncological ventral antigen 2 (NOVA2); Hepatocellular carcinoma antigen gene 520; TUMOR-ASSOClATED ANTIGEN CO-029; Tumor-associated antigens MAGE-C1 (cancer/testis antigen CT7), MAGE-B1 (MAGE-XP antigen), MAGE-B2 (DAM6), MAGE-2, MAGE-4a, MAGE-4b and MAGE-X2; Cancer-Testis Antigen (NY-EOS-1); placental alkaline phosphatase (PLAP) and testicular PLAP-like alkaline phosphatase, transferrin receptor; Heparanase I; EphA2 associated with numerous cancers; DNA/histone H1 complexes that are found in the necrotic cores of many tumor types; amino phospholipids such as phosphatidylserine; Placental Alkaline Phosphatase (PALP); cell surface glycoproteins such as CS1, gp-3, gp4 and gp9 that are associated with numerous tumor types and fragments of any of the above-listed polypeptides.

Other exemplary proteins which may be targeted by and/or incorporated into Fc variant proteins include but not limited to the following list of proteins, as well as subunits, domains, motifs, and epitopes belonging to the following list of microbial proteins: B. anthracis proteins or toxins; human cytomegalovirus (HCMV) proteins such as, envelope glycoprotein, gB, internal matrix proteins of the virus, pp 65 and pp 150, immediate early (1E) proteins; human immunodeficiency virus (HIV) proteins such as, Gag, Pol, Vif and Nef (Vogt et al., 1995, Vaccine 13: 202-208); HIV antigens gp120 and gp160 (Achour et al., 1995, Cell Mol. Biol. 41: 395-400; Hone et al., 1994, Dev. Biol Stand. 82: 159-162); gp41 epitope of human immunodeficiency virus (Eckhart et al., 1996, J. Gen. Virol. 77: 2001-2008); hepatitis C virus (HCV) proteins such as, nucleocapsid protein in a secreted or a nonsecreted form, core protein (pC); E1 (pEL), E2 (pE2) (Saito et al., 1997, Gastroenterology 112: 1321-1330), NS3, NS4a, NS4b and NS5 (Chen et al., 1992, Virology 188:102-113); severe acute respiratory syndrome (SARS) corona virus proteins include but are not limited to, the S (spike) glycoprotein, small envelope protein E (the E protein), the membrane glycoprotein M (the M protein), the hemagglutinin esterase protein (the HE protein), and the nucleocapsid protein (the N-protein) See, e.g., Marra et al., “The Genome Sequence of the SARS-Associated Coronavirus,” Science Express, May 2003); Mycobacterium tuberculosis proteins such as the 30-35 kDa (a.k.a. antigen 85, alpha-antigen) that is normally a lipoglycoprotein on the cell surface, a 65-kDa heat shock protein, and a 36-kDa proline-rich antigen (Tascon et al. (1996) Nat. Med. 2: 888-92), Ag85A, Ag85b (Huygen et al., 1996, Nat. Med. 2: 893-898), 65-kDa heat shock protein, hsp65 (Tascon et al., 1996, Nat. Med. 2: 888-892), MPB/MPT51 (Miki et al., 2004, Infect. Immun. 72:2014-21), MTSP11, MTSP17 (Lim et al., 2004, FEMS Microbiol. Lett. 232:51-9 and supra); Herpes simplex virus (HSV) proteins such as gD glycoprotein, gB glycoprotein; proteins from intracellular parasites such as Leishmania include LPG, gp63 (Xu and Liew, 1994, Vaccine 12: 1534-1536; Xu and Liew, 1995, Immunology 84: 173-176), P-2 (Nylen et al., 2004, Scand. J. Immunol. 59:294-304), P-4 (Kar et al. 2000, J Biol. Chem. 275:37789-97), LACK (Kelly et al., 2003, J Exp. Med. 198:1689-98); microbial toxin proteins such as Clostridium perfringens toxin; C. difficile toxin A and B; in addition, exemplary antigen peptides of human respiratory syncytial virus (hRSV), human metapneumovirus (HMPV) and Parainfluenza virus (PIV) are detailed in: Young et al., in Patent publication WO04010935A2.

One skilled in the art will appreciate that the aforementioned lists of proteins refers not only to specific proteins and biomolecules, but the biochemical pathway or pathways that comprise them. For example, reference to CTLA-4 as a target antigen and/or fusion partner implies that the ligands and receptors that make up the T cell co-stimulatory pathway, including CTLA-4, B7-1, B7-2, CD28, and any other undiscovered ligands or receptors that bind these proteins, are also useful as target antigens and/or fusion partners. Thus, the present invention encompasses not only a specific biomolecule, but the set of proteins that interact with said biomolecule and the members of the biochemical pathway to which said biomolecule belongs. One skilled in the art will also appreciate that antibodies and/or antigen binding fragments thereof, which bind to a protein, the ligands or receptors that bind them, or other members of their corresponding biochemical pathway, may be derived by methods will known in the art, such as those described below, and that such antibodies and/or antigen binding fragments may be engineered to comprise a variant Fc region or fragment thereof including, but not limited to, those described herein. One skilled in the art will further appreciate that any of the aforementioned proteins, the ligands or receptors that bind them, or other members of their corresponding biochemical pathway, may be operably linked to a variant Fc region or fragment thereof including, but not limited to, those described herein in order to generate an Fc fusion. Thus for example, an Fc fusion that targets EGFR could be constructed by operably linking a variant Fc region to EGF, TGFα, or any other ligand, discovered or undiscovered, that binds EGFR. Accordingly, a variant Fc region could be operably linked to EGFR in order to generate an Fc fusion that binds EGF, TGFα, or any other ligand, discovered or undiscovered, that binds EGFR. Thus virtually any polypeptide, whether a ligand, receptor, or some other protein or protein domain, including but not limited to the aforementioned targets and the proteins that compose their corresponding biochemical pathways, may be utilized as a fusion partner to generate an Fc variant protein. It is contemplated that the resulting Fc variant proteins (e.g., antibodies, Fc fusions) targeting and/or incorporating one or more of the molecules listed supra are formulated in accordance with the present invention.

A number of specific multidomain proteins, namely antibodies and antibody domain fusion proteins (e.g., Fc fusions) that are approved for use, in clinical trials, or in development may be modified using methods known in the art to comprise a variant Fc region thereby generating an Fc variant protein. Accordingly, such Fc variant proteins would benefit from the formulations of the present invention. Said antibodies and antibody domain fusion proteins (e.g., Fc fusions) are herein referred to as “clinical products and candidates”. Thus in one embodiment, the formulations of the invention may comprise a range of clinical products and candidates which have been modified to comprise a variant Fc region.

In other embodiments, the formulations of the invention may comprise an Fc variant protein that is derived from a clinical product and/or candidate described herein. For example, the formulations of the invention may comprise an Fc variant protein that comprises at least one, or at least two, or at least three, or at least four, or at least five, or six CDRs from a clinical product and/or candidate. It will be understood by one of skill in the art that a clinical product and/or candidate may be optimized, for example by CDR optimization, to generate a molecule with improved characteristics. Accordingly, other embodiments, the formulations of the invention may comprise an Fc variant protein comprising an amino acid sequence of one or more CDRs that is at least about 80%, or at least about 85%, or at least about 90%, or at least about 92%, or at least about 94%, or at least about 96%, or at least about 98%, or at least about 99%, identical to the amino acid sequence of one or more CDRs from a clinical product and/or candidate. The determination of percent identity of two amino acid sequences can be determined by any method known to one skilled in the art, and described herein, including BLAST protein searches.

In still other embodiments, the Fc variant protein formulations of the invention comprise an Fc variant protein which binds the same antigen as a clinical product and/or candidate. In yet another embodiment, the Fc variant protein formulations of the invention comprise an Fc variant protein which competes for binding to the same antigen as a clinical product and/or candidate. In a specific embodiment, the Fc variant protein present in the formulations of the present invention has binding and functional characteristics substantially similar to a clinical product and/or candidate and comprises, in the Fc region, at least one non naturally occurring amino acid selected from the group consisting of 239D, 330L, 330Y and 332E, as numbered by the EU index as set forth in Kabat For example the formulations of the invention may find use in stabilizing (e.g., reducing aggregation) of an antibody or Fc fusion protein comprising a variant Fc region that has binding and functional characteristics substantially similar to a clinical product and/or candidate including, but not limited to, rituximab (Rituxan®, IDEC/Genentech/Roche) (see for example U.S. Pat. No. 5,736,137), a chimeric anti-CD20 IgG1 antibody approved to treat Non-Hodgkin's lymphoma; zanolimumab (HuMax-CD20, Genmab), an anti-CD20 (see for example PCT WO 04/035607); an anti-CD20 antibody described in U.S. Pat. No. 5,500,362; AME-133 (Applied Molecular Evolution) humanized and optimized anti-CD20 Mab; hA20 (Immunomedics, Inc.) a humanized anti-CD20 Mab; HumaLYM™ (Intracel) a fully human anti-CD20 Mab; anti-CD19 antibodies described in U.S. Pat. Pub. Nos. 2006-0233791, 2006-0263357 and 2006-0280738; anti-CD20 antibodies described in PCT Pat. Pub. Nos. WO 05/000901; anti-CD22 antibodies described in U.S. Pat. No. 5,484,892 and in U.S. Pat. Pub. No. 2003-0202975; trastuzumab (Herceptin®, Genentech) a humanized anti-Her2/neu antibody approved to treat breast cancer (see for example U.S. Pat. No. 5,677,171); pertuzumab (rhuMab-2C4, Omnitarg™, Genentech); an anti-Her2 antibody described in U.S. Pat. No. 4,753,894; cetuximab (Erbitux®, Imclone) (U.S. Pat. No. 4,943,533; PCT WO 96/40210), a chimeric anti-EGFR antibody in clinical trials for a variety of cancers; IMC-3G3 (ImClone), a fully human anti-PDGFRα antibody; panitumumab (Vectibx™, ABX-EGF, Abgenix/Immunex/Amgen), a fully human anti-EGFR antibody described in U.S. Pat. No. 6,235,883; zalutumumab (HuMax-EGFr, Genmab) described in U.S. patent application Ser. No. 10/172,317; EMD55900, EMD62000, and matuzumab (EMD72000, humanized EMD55900) (Merck KGaA) (U.S. Pat. No. 5,558,864), anti-EFGR antibodies; ICR62 (Institute of Cancer Research) (PCT WO 95/20045); nimotuzumab (TheraCIM hR3, YM Biosciences, Canada and Centro de Immunologia Molecular, Cuba) (U.S. Pat. Nos. 5,891,996; 6,506,883); ch806 (humanized mAb-806, Ludwig Institute for Cancer Research, Memorial Sloan-Kettering) (Jungbluth et al. 2003, Proc Natl Acad Sci USA. 100(2):639-44) an anti-EGFR antibody; KSB-102 (KS Biomedix); MR1-1 (IVAX, National Cancer Institute) (PCT WO 01/62931), an affinity optimized anti-EGFR Fvs; and SC100 (Scancell) (PCT WO 01/88138), a deimmunised anti-EGFR antibody; SC101 (Scancell), an anti-Lewis^{y/b} antibody; SC103 (Scancell), an anti-PALP antibody; alemtuzumab (Campath®, Genzyme), a humanized monoclonal anti CD52 IgG1 antibody currently approved for treatment of B-cell chronic lymphocytic leukemia; muromonab-CD3 (Orthoclone OKT3®, Ortho Biotech/Johnson & Johnson), an anti-CD3 antibody; OrthoClone OKT4A (Ortho Biotech), a humanized anti-CD4 IgG antibody; ibritumomab tiuxetan (Zevalin®, IDEC/Schering AG), a radiolabeled anti-CD20 antibody; gemtuzumab ozogamicin (Mylotarg®, (formally, AVE9633, huMy9-6-DM4), Celltech/Wyeth), an anti-CD33 (p67 protein) antibody; alefacept (Amevive®, Biogen), an anti-LFA-3 Fc fusion; abciximab (ReoPro®, Centocor/Lilly), a anti-glycoprotein IIb/IIIa receptor on the platelets for the prevention of clot formation; basiliximab (Simulect®, Novartis) an anti-CD25 antibody; palivizumab (Synagis®, MedImmune), a humanized neutralizing anti-RSV antibody; motavizumab (Numax™, MedImmune), a humanized neutralizing anti-RSV antibody; infliximab (Remicade®, Centocor), an anti-TNFalpha antibody; adalimumab (Humira®, Abbott), an anti-TNFalpha antibody; Humicade™ (CDP-571, CellTech), an anti-TNFalpha antibody; etanercept (Enbrel®, Immunex/Amgen), an anti-TNFalpha Fc fusion; ABX-CBL (Abgenix), an anti-CD147 antibody; ABX-IL8 (Abgenix), an anti-1L8 antibody; ABX-MA1 (Abgenix), an anti-MUC18 antibody; pemtumomab (R1549, ⁹⁰Y-muHMFG1, Antisoma), an anti-MUC1 antibody; Therex (R1550, Antisoma), an anti-MUC1 antibody; AngioMab (AS1405, HuBC-1, Antisoma), an anti-oncofoetal fibronectin antibody and Thioplatin (AS1407) being developed by Antisoma; natalizumab (Antegren®, Biogen), an anti-alpha-4-beta-1 (VLA-4) and alpha-4-beta-7 antibody; ANTOVA™ (IDEC-131, Biogen), a humanized anti-CD40L IgG antibody; VLA-1 mAb (Biogen), an anti-VLA-1 integrin antibody; LTBR mAb (Biogen), an anti-lymphotoxin beta receptor (LTBR) antibody; CAT-152 (Cambridge Antibody Technology), an anti-TGFβ2 antibody; J695 (Cambridge Antibody Technology/Abbott), an anti-IL-12 antibody; CAT-192 (Cambridge Antibody Technology/Genzyme), an anti-TGFβ1 antibody; CAT-213 (Cambridge Antibody Technology), an anti-Eotaxinl antibody; BR3-Fc (BiogenIdec) a soluble BAFF Antagonist; LymphoStat-B™ an anti-Blys antibody and TRAIL-R1mAb, an anti-TRAIL-R1 antibody both being developed by Cambridge Antibody Technology and Human Genome Sciences, Inc.; bevacizumab (Avastin™, rhuMAb-VEGF, Genentech) an anti-VEGF antibody; ranibizumab (Lucentis®, Genentech) an anti-VEGF antibody fragment; an anti-HER receptor family antibody (Genentech); Anti-Tissue Factor antibody (Genentech); omalizumab (Xolair™, Genentech) an anti-IgE antibody; efalizumab (Raptiva™, Genentech/Xoma), an anti-CD11a antibody; MLN-02 Antibody (formerly LDP-02, Genentech/Millenium Pharmaceuticals), a humanized anti-α4β7 antibody; zanolimumab (HuMax CD4, Genmab), an anti-CD4 antibody being; HuMax-IL 15 (Genmab and Amgen), an anti-IL15 antibody; HuMax-Inflam (Genmab/Medarex); HuMax-Cancer (Genmab/Medarex/Oxford GcoSciences), an anti-Heparanase I antibody; HuMax-Lymphoma (Genmab/Amgen); HuMax-TAC (Genmab); clenoliximab (IDEC-151, IDEC Pharmaceuticals), an anti-CD4 antibody; lumiliximab (IDEC-152, IDEC Pharmaceuticals), an anti-CD23; anti-macrophage migration factor (MIF) antibodies being developed by IDEC Pharmaceuticals; BEC2 (Imclone, see U.S. Pat. No. 5,792,455), an anti-idiotypic antibody that mimics GD3; IMC-1C11 (Imclone), an anti-KDR antibody; DC101 (Imclone), an anti-flk-I antibody; anti-VE cadherin antibodies being developed by Imclone; labetuzumab (CEA-Cide™, Immunomedics), an anti-carcinoembryonic antigen (CEA) antibody; arcitumomab (CEAScan®, Immunomedics), an anti-carcinoembryonic antigen (CEA) antibody); epratuzumab (LymphoCide™, Immunomedics), an anti-CD22 antibody; tacatuzumab (AFP-Cide, Immunomedics), a humanized anti-alpha-fetoprotein antibody; MyelomaCide (Immunomedics); LkoCide (Immunomedics); ProstaCide (Immunomedics); ipilimumab (MDX-010, Medarex), an anti-CTLA4 antibody; MDX-060 (Medarex), an anti-CD30 antibody; MDX-070 (Medarex); MDX-018 (Medarex); Valortim™ (MDX-1303, Medarex), an anti-B. anthracis antibody; MDX-1103 (MEDI-545, Medarex/MedImmune) an anti-IFNa antibody; MDX-1333 (MEDI-546, Medarex/MedImmune) an anti-IFNAR antibody; MDX-1106 (ONO-4538, Medarex/Ono Pharmaceutical), an anti-PD1 antibody; MDX-CD4 (Medarex/Eisai/Genmab), a human anti-CD4 IgG antibody; MDX-1388 (MBL/Medarex) a human anti-C. difficile Toxin B antibody; MDX-066 (CDAI, MBL/Medarex), an anti-C. difficile Toxin A antibody; MDX-1307 (Medarex/Celldex), an anti-Mannose Rector (hCGβ) antibody; MDX-214 (Medarex), an anti-EGFR (CD89) antibody; MDX-1100 (Medarex), an anti-IP10 antibody; FG-3019 (Medarex/Fibrogen) an anti-CTGF antibody; HGS-TR2J (Medarex/Kirin) anti-TRAIL-R2; BMS-66513 (Medarex/Bristol-Myers Squibb) an anti-CD137 antibody; SGN-30 (Seattle Genetics) a chimeric anti-CD30 antibody; SGN-40 (Seattle Genetics) a humanized anti-CD40 antibody; tocilizumab (Actemra™, Roche) a humanized anti-IL-6 antibody; CS-1008 (Daiichi Sankyo), a humanized anti-DR5 antibody; Osidemm (IDM-1, Medarex/Immuno-Designed Molecules), an anti-Her2 antibody; golimumab, (CNTO 148, Medarex/Centocor/J&J), an anti-TNFα antibody; CNTO 1275 (Centocor/J&J), an anti-cytokine antibody; CNTO 95 (Centocor/J&J), a human Integrin αᵥ antibody (PCT publication WO 02/12501); CNTO 328 (Centocor/J&J) an anti-IL-6 antibody; mepolizumab (GlaxoSmithKline), a humanized anti-IL-5 antibody; CNTO 528 (Centocor/J&J) an erythropoietic mimetic antibody fusion protein; MOR101 and MOR102 (MorphoSys), anti-intercellular adhesion molecule-1 (ICAM-1) (CD54) antibodies; MOR201 (MorphoSys), an anti-fibroblast growth factor receptor 3 (FGFR-3) antibody; visilizumab (Nuvion®, Protein Design Labs), an anti-CD3 antibody; HuZAF™ (Protein Design Labs), an anti-gamma interferon antibody; volocixmab (M200, Protein Design Labs) a chimeric anti-αVβ3 integrin antibody; anti-IL-12 (Protein Design Labs); HuLuc63 (Protein Design Labs) a humanized anti-CS1 glycoprotein antibody; ING-1 (Xoma), an anti-Ep-CAM antibody; MLN2201 (MLN01, Xoma), an anti-Beta2 integrin antibody; HCD122 (CHIR-12.12, Xoma/Chiron), a fully human anti CD40 antibody; daclizumab (ZENAPAX®, Roche Pharmaceuticals) an immunosuppressive, humanized anti-CD25 monoclonal antibody for the prevention of acute renal allograft rejection; CDP860 (Celltech, UK), a humanized, PEGylated anti-CD18 F(ab′)₂; PRO542 (Progenics/Genzyme Transgenics), an anti-HIV gp120 antibody fused with CD4; C14 (ICOS Pharm), an anti-CD14 antibody; oregovomab (OVAREX™, Altarex), a murine anti-CA 125 antibody; edrecolomab (PANOREX™, Glaxo Wellcome/Centocor), a murine anti-17-IA cell surface antigen IgG2a antibody; etaracizumab (VITAXIN™, MedImmune, PCT publication No. WO 2003/075957), a humanized anti-αVβ3 integrin antibody; siplizumab (MEDI-507, MedImmune), a humanized form of the murine monoclonal anti-CD2 antibody, BTI-322; lintuzumab, (Zamyl™, Smart M195, Protein Design Lab/Kanebo), a humanized anti-CD33 IgG antibody; Remitogen™ (Hu1D10, Protein Design Lab/Kanebo) which is a humanized anti-HLA antibody; ONCOLYM™ (Lym-1, Techniclone) is a radiolabelled murine anti-HLA DR antibody; efalizumab (Genetech/Xoma), a humanized monoclonal anti-CD11a antibody; ICM3 (ICOS Pharm), a humanized anti-ICAM3 antibody; galiximab (IDEC-114, IDEC Pharm/Mitsubishi), a primatized anti-CD80 antibody; eculizumab (5G1.1, Alexion Pharm) a humanized anti-complement factor 5 (C5) antibody; pexelizumab (5G1.1-SC, Alexion Pharm) a fully humanized single chain monoclonal antibody; LDP-01 (Millennium/Xoma), a humanized anti-β₂-integrin IgG antibody; huA33, a fully humanized anti-colonocyte differentiation antigen antibody; Rencarex® (WX-G250, Wilex AG), a murine-human chimeric anti-carbonic anhydrase IX antibody; sibrotuzumab (BIBH 1), a humanized anti-FAPα antibody; Chimeric KW-2871, an anti-GD3 antibody; hu3S193, a humanized anti-Lewis^{Y} blood group antigen antibody; huLK26, a humanized anti-folate binding protein antibody; bivatuzumab (Boehringer/Immunogen) an anti-CD44v6 antibody; ch14.18, a chimeric anti-GD2 antibody; 3F8, a murine anti-GD2 antibody; BC8 a murine anti-CD45 antibody; huHMFG1 a humanized anti-human milk fat globule antibody; MORAb-003 (Morphotek), a humanized anti-GP-3 monoclonal antibody; MORAb-004 (Morphotek), a humanized anti-GP-4 monoclonal antibody; MORAb-009 (Morphotek), a humanized anti-GP-9 monoclonal antibody; denosumab (AMG 162, Amgen) a full human anti RANK ligand antibody; PRO-140 (Progenics) an anti-CCR5 antibody; 1D09C3 (GPC Biotech/Morphosys) a fully human anti-HLA-DR IgG4 antibody; huMikbeta-1 a humanized anti-IL-2R/IL-15R beta subunit (CD122) antibody; NI-0401 (NovImmune) an anti-CD3 antibody; NI-501 (NovImmune) an anti-IFN-gamma antibody; cantuzumab mertansine (HuC242, ImmunoGen Inc) anti-CanAg antigen antibody; HuN901 (ImmunoGen Inc) anti-CD56 antibody; 8H9 antibody as described in U.S. Patent Publication 2003/0103963; chTNT-1/B (Peregrine) a chimeric anti-DNA/histone H1 complex antibody; bavituximab (Peregrine) a chimeric anti-phosphatidylserine antibody; huJ591, a de-immunized anti-PSMA antibody; HeFi-1, a mouse anti-CD30 antibody; Pentacea™ (IBC Pharmaceuticals) an anti-CEA×anti-DTPA-indium bispecific antibody; abagovomab (MEL-1 and MEL-2, MELIMMUNE™), a combination of murine anti-idiotype antibody against CA125; 105AD7 (Onyvac-P, CRC Technology/Oncovac) idiotypic antibody which mimics CD55 (Gp72); tositumomab (BEXXAR™, Corixa/GSK); GMA161 (Macrogenics) an anti-FcγRIIIA (CD16A) antibody and GMA321 (Macrogenics) an anti-FcγRIIB (CD32B) antibody; anti-CD16A/CD32B diabody-Fc fusion molecule described in U.S. Pat. Pub. 2007/0004909.

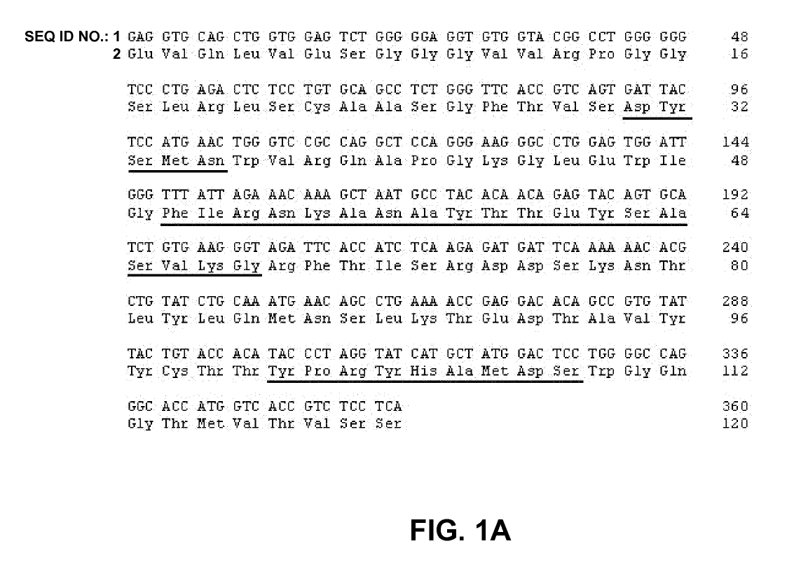

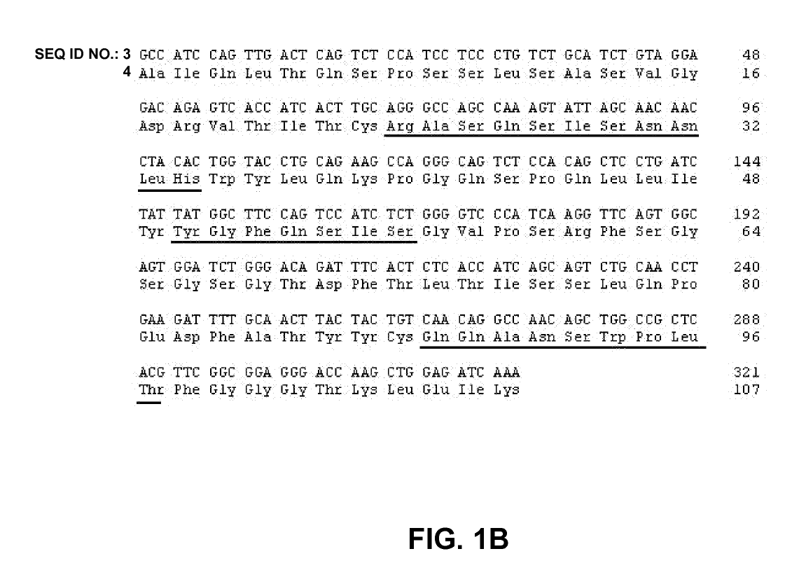

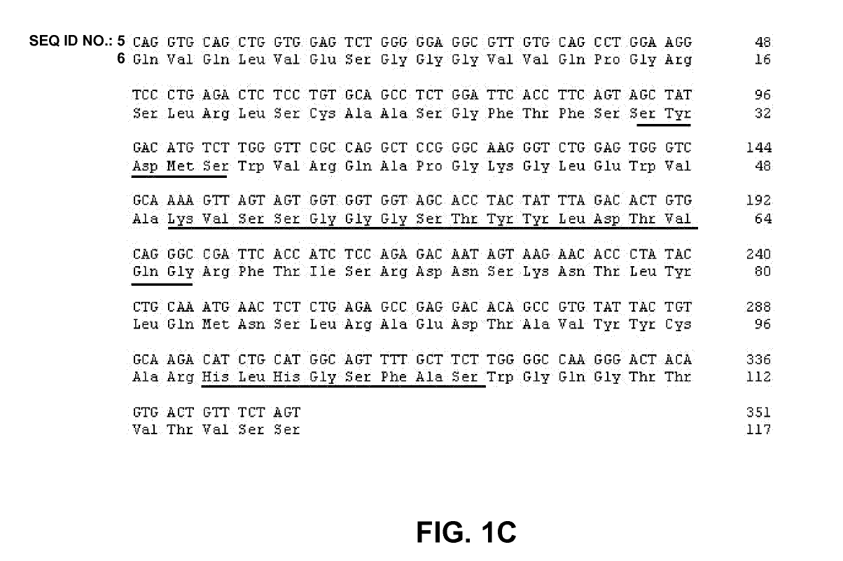

In one embodiment, the Fc variant protein formulations of the invention comprise an Fc variant protein derived from an antibody or other protein (e.g., Fc fusion protein) that binds to a member of the receptor tyrosine kinase family or a ligand thereof. Members of the receptor tyrosine kinase family include but are not limited to, members of the Eph family of receptors (e.g., EphA1, EphA2, EphA3, EphA4, EphA5, EphA6, EphA7, EphA8, EphB1, EphB2, EphB3, EphB4, EphB5, EphB6), ALK. Ligands of the receptor tyrosine kinase family include, but are not limited to, member of the ephrin ligands (e.g., ephrinA1, ephrinA2, ephrinA4, ephrinA5, ephrinB1, ephrinB2, ephrinB3 and pleotropin). In certain embodiments, the antibody or other protein binds EphA2, EphA4, EphB4 or ALK. In other embodiments, the antibody or other protein binds a ligand of EphA2, EphA4, EphB4 or ALK. Exemplary antibodies and other proteins which bind EphA2, EphA4, EphB4, ALK or ligands thereof are disclosed in U.S. patent application Ser. No. 11/203,251, PCT Patent Application No. PCT/US2006/044637 and Patent Publication Nos. WO 06/034456 and WO 06/034455. In a specific embodiment, the Fc variant protein formulations of the invention comprise an Fc variant protein that binds EphA2, wherein said Fc variant protein comprises at least 1, at least 2, at least 3, at least 4, at least 5, or at least 6 CDRs of the Medi3 variable domain (see, FIGS. 1A-1B). In another specific embodiment, the Fc variant protein that binds EphA2, comprises in the Fc region, at least one non naturally occurring amino acid selected from the group consisting of 239D, 330L, 330Y and 332E, as numbered by the EU index as set forth in Kabat.

In one embodiment, the Fc variant protein formulations of the invention comprise an Fc variant protein derived from an antibody or other protein (e.g., Fc fusion protein) that binds to an integrin subunit and/or combination thereof. Members of the integrin subunits include, but are not limited to, integrin alpha subunits such as CD49a, CD49b, CD49c, CD49d, CD49e, CD49f, alpha7, alpha8, alpha9, alphaD, CD11a, CD11b, CD51, CD11c, CD41, alphaIIb, alphaIELb; integrin beta subunits such as, CD29, CD 18, CD61, CD104, beta5, beta6, beta7 and beta8. Exemplary, integrin subunit combinations include, but not are limited to, αVβ3, αVβ5 and α4β7. In a specific embodiment, the antibody or other protein binds αV, β3 and/or αVβ3. Exemplary antibodies and other proteins which bind αV, β3 and/or αVβ3 are disclosed in U.S. patent application Ser. No. 11/203,253. In a specific embodiment, the Fc variant protein formulations of the invention comprise an Fc variant protein that binds integrin αVβ3, wherein said Fc variant protein comprises at least 1, at least 2, at least 3, at least 4, at least 5, or at least 6 CDRs of the Medi2 variable domain (see, FIGS. 1C-1D). In another specific embodiment, the Fc variant protein that binds integrin αVβ3, comprises in the Fc region, at least one non naturally occurring amino acid selected from the group consisting of 239D, 330L, 330Y and 332E, as numbered by the EU index as set forth in Kabat.

The percent identity of two amino acid sequences (or two nucleic acid sequences) can be determined, for example, by aligning the sequences for optimal comparison purposes (e.g., gaps can be introduced in the sequence of a first sequence). The amino acids or nucleotides at corresponding positions are then compared, and the percent identity between the two sequences is a function of the number of identical positions shared by the sequences (i.e., % identity=# of identical positions/total # of positions×100). The actual comparison of the two sequences can be accomplished by well-known methods, for example, using a mathematical algorithm. A specific, non-limiting example of such a mathematical algorithm is described in Karlin et al., Proc. Natl. Acad. Sci. USA, 90:5873-5877 (1993). Such an algorithm is incorporated into the BLASTN and BLASTX programs (version 2.2) as described in Schaffer et al., Nucleic Acids Res., 29:2994-3005 (2001). When utilizing BLAST and Gapped BLAST programs, the default parameters of the respective programs (e.g., BLASTN) can be used. See http://www.ncbi.nlm.nih.gov, as available on Apr. 10, 2002. In one embodiment, the database searched is a non-redundant (NR) database, and parameters for sequence comparison can be set at: no filters; Expect value of 10; Word Size of 3; the Matrix is BLOSUM62; and Gap Costs have an Existence of 11 and an Extension of 1.

Another, non-limiting example of a mathematical algorithm utilized for the comparison of sequences is the algorithm of Myers and Miller, CABIOS (1989). Such an algorithm is incorporated into the ALIGN program (version 2.0), which is part of the GCG (Accelrys) sequence alignment software package. When utilizing the ALIGN program for comparing amino acid sequences, a PAM 120 weight residue table, a gap length penalty of 12, and a gap penalty of 4 can be used. Additional algorithms for sequence analysis are known in the art and include ADVANCE and ADAM as described in Torellis and Robotti, Comput. Appl. Biosci., 10: 3-5 (1994); and FASTA described in Pearson and Lipman, Proc. Natl. Acad. Sci. USA, 85: 2444-8 (1988).

In another embodiment, the percent identity between two amino acid sequences can be accomplished using the GAP program in the GCG software package (available at http://www.accelrys.com, as available on Aug. 31, 2001) using either a Blossom 63 matrix or a PAM250 matrix, and a gap weight of 12, 10, 8, 6, or 4 and a length weight of 2, 3, or 4. In yet another embodiment, the percent identity between two nucleic acid sequences can be accomplished using the GAP program in the GCG software package (available at http://www.cgc.com), using a gap weight of 50 and a length weight of 3.

### 6.4.4 Fc Variant Protein Conjugates And Derivatives

Also encompassed by the formulations the invention are Fc variant protein derivatives which are Fc variant proteins that are modified by the attachment of any type of molecule, including but not limited to, peptides, polypeptides, proteins, fusion proteins, nucleic acid molecules, small molecules, mimetic agents, synthetic drugs, inorganic molecules, and organic molecules. For example, but not by way of limitation, the Fc variant protein derivatives include Fc variant proteins that have been modified, e.g., by glycosylation, acetylation, pegylation, phosphorylation, amidation, derivatization by known protecting/blocking groups, proteolytic cleavage, linkage to a cellular ligand or other protein, etc. Any of numerous chemical modifications may be carried out by known techniques, including, but not limited to, specific chemical cleavage, acetylation, formylation, metabolic synthesis of tunicamycin, etc. Additionally, the derivative may contain one or more non-classical amino acids.

Fc variant proteins with increased in vivo half-lives can be generated by attaching to said antibodies or antibody fragments polymer molecules such as high molecular weight polyethyleneglycol (PEG). PEG can be attached to said antibodies or antibody fragments with or without a multifunctional linker either through site-specific conjugation of the PEG to the N- or C-terminus of said antibodies or antibody fragments or via epsilon-amino groups present on lysine residues. Linear or branched polymer derivatization that results in minimal loss of biological activity will be used. The degree of conjugation will be closely monitored by SDS-PAGE and mass spectrometry to ensure proper conjugation of PEG molecules to the antibodies. Unreacted PEG can be separated from antibody-PEG conjugates by, e.g., size exclusion or ion-exchange chromatography.

In one embodiment, the present invention encompasses formulations comprising Fc variant proteins recombinantly fused or chemically conjugated (including both covalent and non-covalent conjugations) to a heterologous protein or polypeptide (or fragment thereof, preferably to a polypeptide of at least 10, at least 20, at least 30, at least 40, at least 50, at least 60, at least 70, at least 80, at least 90 or at least 100 amino acids). For example, Fc variant proteins may be used to target heterologous polypeptides to particular cell types, either in vitro or in vivo, by fusing or conjugating the Fc variant proteins to antibodies specific for particular cell surface receptors. Fc variant proteins fused or conjugated to heterologous polypeptides may also be used in in vitro immunoassays and purification methods using methods known in the art. See e.g., International publication No. WO 93/21232; European Patent No. EP 439,095; Naramura et al., 1994, Immunol. Lett. 39:91-99; U.S. Pat. No. 5,474,981; Gillies et al., 1992, PNAS 89:1428-1432; and Fell et al., 1991, J. Immunol. 146:2446-2452.

Further, Fc variant proteins can be conjugated to albumin in order to make the Fc variant protein more stable in vivo or have a longer half life in vivo. The techniques are well known in the art, see e.g., International Publication Nos. WO 93/15199, WO 93/15200, and WO 01/77137; and European Patent No. EP 413, 622.

Moreover, Fc variant proteins can be fused to marker sequences, such as a peptide to facilitate purification. In certain embodiments, the marker amino acid sequence is a hexa-histidine peptide, such as the tag provided in a pQE vector (QIAGEN, Inc., 9259 Eton Avenue, Chatsworth, Calif., 91311), among others, many of which are commercially available. As described in Gentz et al., 1989, Proc. Natl. Acad. Sci. USA 86:821-824, for instance, hexa-histidine provides for convenient purification of the fusion protein. Other peptide tags useful for purification include, but are not limited to, the hemagglutinin “HA” tag, which corresponds to an epitope derived from the influenza hemagglutinin protein (Wilson et al., 1984, Cell 37:767) and the “flag” tag.

In other embodiments, Fc variant proteins or analogs or derivatives thereof are conjugated to a diagnostic or detectable agent. Such Fc variant proteins can be useful for monitoring or prognosing the development or progression of a disease as part of a clinical testing procedure, such as determining the efficacy of a particular therapy. Such diagnosis and detection can be accomplished by coupling the Fc variant protein to detectable substances including, but not limited to various enzymes, such as but not limited to horseradish peroxidase, alkaline phosphatase, beta-galactosidase, or acetylcholinesterase; prosthetic groups, such as but not limited to streptavidin/biotin and avidin/biotin; fluorescent materials, such as but not limited to, umbelliferone, fluorescein, fluorescein isothiocynate, rhodamine, dichlorotriazinylamine fluorescein, dansyl chloride or phycoerythrin; luminescent materials, such as but not limited to, luminol; bioluminescent materials, such as but not limited to, luciferase, luciferin, and aequorin; radioactive materials, such as but not limited to iodine (131I, 125I, 123I, 121I), carbon (14C), sulfur (35S), tritium (3H), indium (115In, 113In, 112In, 111In), and technetium (99Tc), thallium (201Ti), gallium (68Ga, 67Ga), palladium (103Pd), molybdenum (99Mo), xenon (133Xe), fluorine (18F), 153Sm, 177Lu, 159Gd, 149 Pm, 140La, 175Yb, 166Ho, 90Y, 47Sc, 186Re, 188Re, 142 Pr, 105Rh, 97Ru, 68Ge, 57Co, 65Zn, 85Sr, 32P, 153Gd, 169Yb, 51Cr, 54Mn, 75Se, 113Sn, and 117Tin; positron emitting metals using various positron emission tomographies, noradioactive paramagnetic metal ions, and molecules that are radiolabelled or conjugated to specific radioisotopes.

The present invention further encompasses Fc variant proteins conjugated to a therapeutic agent. An Fc variant protein may be conjugated to a therapeutic moiety such as a cytotoxin, e.g., a cytostatic or cytocidal agent, a therapeutic agent or a radioactive metal ion, e.g., alpha-emitters. A cytotoxin or cytotoxic agent includes any agent that is detrimental to cells. Examples include paclitaxel, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, puromycin, epirubicin, and cyclophosphamide and analogs or homologs thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BCNU) and lomustine (CCNU), cyclothosphamide, busulfan, dibromomannitol, streptozotocin, mitomycin C, and cisdichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine). A more extensive list of therapeutic moieties can be found in PCT publications WO 03/075957.

Further, an Fc variant protein may be conjugated to a therapeutic agent or drug moiety that modifies a given biological response. Therapeutic agents or drug moieties are not to be construed as limited to classical chemical therapeutic agents. For example, the drug moiety may be a protein or polypeptide possessing a desired biological activity. Such proteins may include, for example, a toxin such as abrin, ricin A, Onconase (or another cytotoxic RNase), pseudomonas exotoxin, cholera toxin, or diphtheria toxin; a protein such as tumor necrosis factor, α-interferon, β-interferon, nerve growth factor, platelet derived growth factor, tissue plasminogen activator, an apoptotic agent, e.g., TNF-α, TNF-β, AIM I (see, International Publication No. WO 97/33899), AIM II (see, International Publication No. WO 97/34911), Fas Ligand (Takahashi et al., 1994, J. Immunol., 6:1567), and VEGI (see, International Publication No. WO 99/23105), a thrombotic agent or an anti-angiogenic agent, e.g., angiostatin or endostatin; or, a biological response modifier such as, for example, a lymphokine (e.g., interleukin-1 (“IL-1”), interleukin-2 (“IL-2”), interleukin-6 (“IL-6”), granulocyte macrophage colony stimulating factor (“GM-CSF”), and granulocyte colony stimulating factor (“G-CSF”)), or a growth factor (e.g., growth hormone (“GH”)).

Moreover, an Fc variant protein can be conjugated to therapeutic moieties such as radioactive materials or macrocyclic chelators useful for conjugating radiometal ions (see above for examples of radioactive materials). In certain embodiments, the macrocyclic chelator is 1,4,7,10-tetraazacyclododecane-N,N′,N″,N″-tetraacetic acid (DOTA) which can be attached to the Fc variant protein via a linker molecule. Such linker molecules are commonly known in the art and described in Denardo et al., 1998, Clin Cancer Res. 4:2483; Peterson et al., 1999, Bioconjug. Chem. 10:553; and Zimmerman et al., 1999, Nucl. Med. Biol. 26:943.

Techniques for conjugating therapeutic moieties to antibodies are well known. Moieties can be conjugated to antibodies (e.g., Fc variant protein) by any method known in the art, including, but not limited to aldehyde/Schiff linkage, sulphydryl linkage, acid-labile linkage, cis-aconityl linkage, hydrazone linkage, enzymatically degradable linkage (see generally Garnett, 2002, Adv Drug Deliv Rev 53:171). Methods for fusing or conjugating antibodies to polypeptide moieties are known in the art. See, e.g., U.S. Pat. Nos. 5,336,603; 5,622,929; 5,359,046; 5,349,053; 5,447,851, and 5,112,946; EP 307,434; EP 367,166; PCT Publications WO 96/04388 and WO 91/06570; Ashkenazi et al., 1991, PNAS USA 88:10535; Zheng et al., 1995, J Immunol 154:5590; and Vil et al., 1992, PNAS USA 89:11337. The fusion of an antibody to a moiety does not necessarily need to be direct, but may occur through linker sequences. Such linker molecules are commonly known in the art and described in Denardo et al., 1998, Clin Cancer Res 4:2483; Peterson et al., 1999, Bioconjug Chem 10:553; Zimmerman et al., 1999, Nucl Med Biol 26:943; Garnett, 2002, Adv Drug Deliv Rev 53:171. These methods may also be utilized for conjugation of therapeutic moieties to Fc fusion proteins.

### 6.5 Methods of Generating Fc Variant Proteins

### 6.5.1 Generating Antibodies

The formulations of the invention are useful for antibodies produced by any method known in the art for the synthesis of antibodies, in particular, by chemical synthesis or by recombinant expression techniques. In certain embodiments the formulations of the present invention comprise antibodies, wherein said antibodies comprise variant Fc regions.

Polyclonal antibodies recognizing a particular antigen can be produced by various procedures well known in the art. For example, an antigen or immunogenic fragments thereof can be administered to various host animals including, but not limited to, rabbits, mice, rats, etc. to induce the production of sera containing polyclonal antibodies specific for an antigen. Various adjuvants may be used to increase the immunological response, depending on the host species, and include but are not limited to, Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and corynebacterium parvum. Such adjuvants are also well known in the art.

Monoclonal antibodies can be prepared using a wide variety of techniques known in the art including the use of hybridoma, recombinant, and phage display technologies, or a combination thereof. For example, monoclonal antibodies can be produced using hybridoma techniques including those known in the art and taught, for example, in Harlow et al., Antibodies: A Laboratory Manual, (Cold Spring Harbor Laboratory Press, 2nd ed. 1988); Hammerling, et al., in: Monoclonal Antibodies and T-Cell Hybridomas 563-681 (Elsevier, N.Y., 1981). The term “monoclonal antibody” as used herein is not limited to antibodies produced through hybridoma technology. The term “monoclonal antibody” refers to an antibody that is derived from a single clone, including any eukaryotic, prokaryotic, or phage clone, and not the method by which it is produced.

Methods for producing and screening for specific antibodies using hybridoma technology are routine and well known in the art. Briefly, mice can be immunized with an antigen or immunogenic fragment thereof and once an immune response is detected, e.g., antibodies specific for the administered antigen are detected in the mouse serum, the mouse spleen is harvested and splenocytes isolated. The splenocytes are then fused by well known techniques to any suitable myeloma cells, for example cells from cell line SP20 available from the ATCC. Additionally, a RIMMS (repetitive immunization, multiple sites) technique can be used to immunize an animal (Kilpatrick et al., 1997, Hybridoma 16:381-9). Hybridomas are selected and cloned by limited dilution. The hybridoma clones are then assayed by methods known in the art for cells that secrete antibodies capable of binding a polypeptide of the invention. Ascites fluid, which generally contains high levels of antibodies, can be generated by immunizing mice with positive hybridoma clones.

Accordingly, monoclonal antibodies can be generated by culturing a hybridoma cell secreting an antibody wherein, the hybridoma may be generated by fusing splenocytes isolated from a mouse immunized with an antigen or immunogenic fragments thereof, with myeloma cells and then screening the hybridomas resulting from the fusion for hybridoma clones that secrete an antibody able to bind the administered antigen.

The formulations of the present invention are useful for stabilizing antibodies comprising variant Fc regions or fragments thereof. Antibodies comprising variant Fc regions can be generated by numerous methods well known to one skilled in the art. Non-limiting examples include, isolating antibody coding regions (e.g., from hybridoma) and introducing one or more modifications into the Fc region of the isolated antibody coding region. Alternatively, the variable regions may be subcloned into a vector encoding a variant Fc region or fragment thereof including, but not limited to, those described herein. Additional methods and details are provided below.

Antibody fragments that recognize specific an antigen may be generated by any technique known to those of skill in the art. For example, Fab and F(ab′)2 fragments of the invention may be produced by proteolytic cleavage of immunoglobulin molecules, using enzymes such as papain (to produce Fab fragments) or pepsin (to produce F(ab′)2 fragments). F(ab′)2 fragments contain the variable region, the light chain constant region and the CH1 domain of the heavy chain. Further, the antibodies of the present invention can also be generated using various phage display methods known in the art.

In phage display methods, functional antibody domains are displayed on the surface of phage particles that carry the polynucleotide sequences encoding them. In particular, DNA sequences encoding VH and VL domains are amplified from animal cDNA libraries (e.g., human or murine cDNA libraries of lymphoid tissues). The DNA encoding the VH and VL domains are recombined together with an scFv linker by PCR and cloned into a phagemid vector (e.g., p CANTAB 6 or pComb 3 HSS). The vector is electroporated in E. coli and the E. coli is infected with helper phage. Phage used in these methods are typically filamentous phage including fd and M13 and the VH and VL domains are usually recombinantly fused to either the phage gene III or gene VIII. Phage expressing an antigen binding domain that binds to the an Antigen epitope of interest can be selected or identified with antigen, e.g., using labeled antigen or antigen bound or captured to a solid surface or bead. Examples of phage display methods that can be used to make the antibodies of the present invention include those disclosed in Brinkman et al., 1995, J. Immunol. Methods 182:41-50; Ames et al., 1995, J. Immunol. Methods 184:177-186; Kettleborough et al., 1994, Eur. J. Immunol. 24:952-958; Persic et al., 1997, Gene 187:9-18; Burton et al., 1994, Advances in Immunology 57:191-280; PCT Publication Nos. WO 90/02809, WO 91/10737, WO 92/01047, WO 92/18619, WO 93/11236, WO 95/15982, WO 95/20401, and WO97/13844; and U.S. Pat. Nos. 5,698,426, 5,223,409, 5,403,484, 5,580,717, 5,427,908, 5,750,753, 5,821,047, 5,571,698, 5,427,908, 5,516,637, 5,780,225, 5,658,727, 5,733,743 and 5,969,108.

As described in the above references, after phage selection, the antibody coding regions from the phage can be isolated and used to generate whole antibodies, including human antibodies, or any other desired antigen binding fragment, and expressed in any desired host, including mammalian cells, insect cells, plant cells, yeast, and bacteria, e.g., as described below. Techniques to recombinantly produce Fab, Fab′ and F(ab′)2 fragments can also be employed using methods known in the art such as those disclosed in International Publication No. WO 92/22324; Mullinax et al., 1992, BioTechniques 12(6): 864-869; Sawai et al., 1995, AJRI 34:26-34; and Better et al., 1988, Science 240:1041-1043.

To generate whole antibodies, PCR primers including VH or VL nucleotide sequences, a restriction site, and a flanking sequence to protect the restriction site can be used to amplify the VH or VL sequences in scFv clones. Utilizing cloning techniques known to those of skill in the art, the PCR amplified VH domains can be cloned into vectors expressing a VH constant region, e.g., the human gamma constant, and the PCR amplified VL domains can be cloned into vectors expressing a VL constant region, e.g., human kappa or lamba constant regions. It is contemplated that the heavy chain constant region comprises or alternatively consists of a variant Fc region including, but not limited to, those disclosed herein. In certain embodiments, the vectors for expressing the VH or VL domains comprise a promoter, a secretion signal, a cloning site for both the variable and constant domains, as well as a selection marker such as neomycin. The VH and VL domains may also be cloned into one vector expressing the desired constant regions. The heavy chain conversion vectors and light chain conversion vectors are then co-transfected into cell lines to generate stable or transient cell lines that express full-length antibodies, e.g., IgG, using techniques known to those of skill in the art.

Phage display technology can also be utilized to select antibody genes with binding activities towards an antigen either from repertoires of PCR amplified v-genes of lymphocytes from humans screened for possessing antigen binding antibodies or from naive libraries (McCafferty et al., Nature 348:552-554, 1990; and Marks, et al., Biotechnology 10:779-783, 1992). The affinity of these antibodies can also be improved by chain shuffling (Clackson et al., Nature 352: 624-628, 1991). Related techniques have been described for antibody optimization (see, e.g., Wu & An, 2003, Methods Mol. Biol., 207, 213-233; Wu, 2003, Methods Mol. Biol., 207, 197-212; and Kunkel et al., 1987, Methods Enzymol. 154, 367-382).

A chimeric antibody is a molecule in which different portions of the antibody are derived from different immunoglobulin molecules. Methods for producing chimeric antibodies are known in the art. See e.g., Morrison, 1985, Science 229:1202; Oi et al., 1986, BioTechniques 4:214; Gillies et al., 1989, J. Immunol. Methods 125:191-202; and U.S. Pat. Nos. 5,807,715, 4,816,567, 4,816,397, and 6,311,415.

For some uses, including in vivo use of antibodies in humans and in vitro detection assays, it may be preferable to use human or chimeric antibodies. Completely human antibodies are particularly desirable for therapeutic treatment of human subjects. Human antibodies can be made by a variety of methods known in the art including phage display methods described above using antibody libraries derived from human immunoglobulin sequences. See also U.S. Pat. Nos. 4,444,887 and 4,716,111; and PCT Publication Nos. WO 05/042743; WO 98/46645; WO 98/50433; WO 98/24893; WO98/16654; WO 96/34096, WO 96/33735, and WO 91/10741.

If the antibody is used therapeutically in in vivo applications, the antibody is preferably modified to make it less immunogenic in the individual. For example, if the individual is human the antibody is preferably “humanized”; where the complementarity determining region(s) of the antibody is transplanted into a human antibody (for example, as described in Jones et al., Nature 321:522-525, 1986; and Tempest et al., Biotechnology 9:266-273, 1991).

A humanized antibody is an antibody or its variant or fragment thereof which is capable of binding to a predetermined antigen and which comprises a framework region having substantially the amino acid sequence of a human immunoglobulin and a CDR having substantially the amino acid sequence of a non-human immunoglobulin. A humanized antibody comprises substantially all of at least one, and typically two, variable domains (Fab, Fab′, F(ab′)₂, Fabc, Fv) in which all or substantially all of the CDR regions correspond to those of a non-human immunoglobulin (i.e., donor antibody) and all or substantially all of the framework regions are those of a human immunoglobulin consensus sequence. In a specific embodiment, a humanized antibody also comprises at least a portion of an immunoglobulin constant region (Fc), typically that of a human immunoglobulin. Ordinarily, the antibody will contain both the light chain as well as at least the variable domain of a heavy chain. The antibody also may include the CH1, hinge, CH2, CH3, and CH4 regions of the heavy chain. The humanized antibody can be selected from any class of immunoglobulins, including IgM, IgG, IgD, IgA and IgE, and any isotype, including IgG1, IgG2, IgG3 and IgG4. Usually the constant domain is a complement fixing constant domain where it is desired that the humanized antibody exhibit cytotoxic activity, and the class is typically IgG.sub. 1. Where such cytotoxic activity is not desirable, the constant domain may be of the IgG.sub.2 class. The humanized antibody may comprise sequences from more than one class or isotype. Furthermore, as described herein, selecting particular constant domain comprising variant Fc regions to optimize desired effector functions is within the ordinary skill in the art. The framework and CDR regions of a humanized antibody need not correspond precisely to the parental sequences, e.g., the donor CDR or the consensus framework may be mutagenized by substitution, insertion or deletion of at least one residue so that the CDR or framework residue at that site does not correspond to either the consensus or the import antibody. Such mutations, however, will not be extensive. Usually, at least 75% of the humanized antibody residues will correspond to those of the parental framework region (FR) and CDR sequences, more often 90%, or greater than 95%. Humanized antibody can be produced using variety of techniques known in the art, including but not limited to, CDR-grafting (European Patent No. EP 239,400; International Publication No. WO 91/09967; and U.S. Pat. Nos. 5,225,539, 5,530,101, and 5,585,089), veneering or resurfacing (European Patent Nos. EP 592,106 and EP 519,596; Padlan, 1991, Molecular Immunology 28(4/5): 489-498; Studnicka et al., 1994, Protein Engineering 7(6): 805-814; and Roguska et al., 1994, PNAS 91:969-973), chain shuffling (U.S. Pat. No. 5,565,332), framework shuffling (International Publication No. WO 05/042743) and techniques disclosed in, e.g., U.S. Pat. No. 6,407,213, U.S. Pat. No. 5,766,886, WO 9317105, Tan et al., J. Immunol. 169:1119-25 (2002), Caldas et al., Protein Eng. 13(5): 353-60 (2000), Morea et al., Methods 20(3): 267-79 (2000), Baca et al., J. Biol. Chem. 272(16): 10678-84 (1997), Roguska et al., Protein Eng. 9(10): 895-904 (1996), Couto et al., Cancer Res. 55 (23 Supp): 5973s-5977s (1995), Couto et al., Cancer Res. 55(8): 1717-22 (1995), Sandhu J S, Gene 150(2): 409-10 (1994), and Pedersen et al., J. Mol. Biol. 235(3): 959-73 (1994). Often, framework residues in the framework regions will be substituted with the corresponding residue from the CDR donor antibody to alter, preferably improve, antigen binding. These framework substitutions are identified by methods well known in the art, e.g., by modeling of the interactions of the CDR and framework residues to identify framework residues important for antigen binding and sequence comparison to identify unusual framework residues at particular positions. (See, e.g., Queen et al., U.S. Pat. No. 5,585,089; and Riechmann et al., 1988, Nature 332:323).

Human antibodies can also be produced using transgenic mice which are incapable of expressing functional endogenous immunoglobulins, but which can express human immunoglobulin genes. For example, the human heavy and light chain immunoglobulin gene complexes may be introduced randomly or by homologous recombination into mouse embryonic stem cells. Alternatively, the human variable region, constant region, and diversity region may be introduced into mouse embryonic stem cells in addition to the human heavy and light chain genes. The mouse heavy and light chain immunoglobulin genes may be rendered non-functional separately or simultaneously with the introduction of human immunoglobulin loci by homologous recombination. In particular, homozygous deletion of the JH region prevents endogenous antibody production. The modified embryonic stem cells are expanded and microinjected into blastocysts to produce chimeric mice. The chimeric mice are then bred to produce homozygous offspring that express human antibodies. The transgenic mice are immunized in the normal fashion with a selected antigen or immunogenic fragments thereof. Monoclonal antibodies directed against the antigen can be obtained from the immunized, transgenic mice using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA, IgM and IgE antibodies. For an overview of this technology for producing human antibodies, see Lonberg and Huszar (1995, Int. Rev. Immunol. 13:65-93). For a detailed discussion of this technology for producing human antibodies and human monoclonal antibodies and protocols for producing such antibodies, see, e.g., International Publication Nos. WO 98/24893, WO 96/34096, and WO 96/33735; and U.S. Pat. Nos. 5,413,923, 5,625,126, 5,633,425, 5,569,825, 5,661,016, 5,545,806, 5,814,318, and 5,939,598. In addition, companies such as Abgenix, Inc. (Freemont, Calif.), Genpharm (San Jose, Calif.) and Medarex (Princeton, N.J.) can be engaged to provide human antibodies directed against a selected antigen using technology similar to that described above.

Completely human antibodies which recognize a selected epitope can be generated using a technique referred to as “guided selection.” In this approach a selected non-human monoclonal antibody, e.g., a mouse antibody, is used to guide the selection of a completely human antibody recognizing the same epitope. (Jespers et al., Bio/technology 12:899-903 (1988)).

Further, the antibodies of the invention can, in turn, be utilized to generate anti-idiotype antibodies that “mimic” a polypeptide using techniques well known to those skilled in the art. (See, e.g., Greenspan & Bona, 1989, FASEB J. 7(5): 437-444; and Nissinoff, 1991, J. Immunol. 147(8): 2429-2438). For example, antibodies of the invention which bind to and competitively inhibit the binding of a polypeptide (as determined by assays well known in the art and disclosed infra) to a binding partner (e.g., a ligand or receptor) can be used to generate anti-idiotypes that “mimic” the polypeptide and, as a consequence, bind to and neutralize binding partner (e.g., the receptor and/or its ligands). Such neutralizing anti-idiotypes or Fab fragments of such anti-idiotypes can be used in therapeutic regimens to neutralize a binding partner of a polypeptide.

In one embodiment, the nucleotide sequence encoding an antibody that specifically binds an antigen is obtained and used to generate the Fc variant proteins of the invention. The nucleotide sequence can be obtained from sequencing hybridoma clone DNA. If a clone containing a nucleic acid encoding a particular antibody or an epitope-binding fragment thereof is not available, but the sequence of the antibody molecule or epitope-binding fragment thereof is known, a nucleic acid encoding the immunoglobulin may be chemically synthesized or obtained from a suitable source (e.g., an antibody cDNA library, or a cDNA library generated from, or nucleic acid, preferably poly A+RNA, isolated from any tissue or cells expressing the antibody, such as hybridoma cells selected to express an antibody) by PCR amplification using synthetic primers that hybridize to the 3′ and 5′ ends of the sequence or by cloning using an oligonucleotide probe specific for the particular gene sequence to identify, e.g., a cDNA clone from a cDNA library that encodes the antibody. Amplified nucleic acids generated by PCR may then be cloned into replicable cloning vectors using any method well known in the art.

Once the nucleotide sequence of the antibody is determined, the nucleotide sequence of the antibody may be manipulated using methods well known in the art for the manipulation of nucleotide sequences, e.g., recombinant DNA techniques, site directed mutagenesis, PCR, etc. (see, for example, the techniques described in Current Protocols in Molecular Biology, F. M. Ausubel et al., ed., John Wiley & Sons (Chichester, England, 1998); Molecular Cloning: A Laboratory Manual, 3rd Edition, J. Sambrook et al., ed., Cold Spring Harbor Laboratory Press (Cold Spring Harbor, N.Y., 2001); Antibodies: A Laboratory Manual, E. Harlow and D. Lane, ed., Cold Spring Harbor Laboratory Press (Cold Spring Harbor, N.Y., 1988); and Using Antibodies: A Laboratory Manual, E. Harlow and D. Lane, ed., Cold Spring Harbor Laboratory (Cold Spring Harbor, N.Y., 1999)), to generate antibodies having a different amino acid sequence by, for example, introducing deletions, and/or insertions into desired regions of the antibodies.

In one embodiment, one or more modification is made within the Fc region (e.g. supra) of an antibody able to specifically bind an antigen. It is specifically contemplated that the modification alters binding to at least one Fc ligand (e.g., FcγRs and/or C1q) and/or alters ADCC and/or CDC function.

In a specific embodiment, one or more of the CDRs is inserted within framework regions using routine recombinant DNA techniques. The framework regions may be naturally occurring or consensus framework regions, including, but not limited to, human framework regions (see, e.g., Chothia et al., 1998, J. Mol. Biol. 278: 457-479 for a listing of human framework regions). It is contemplated that the polynucleotide generated by the combination of the framework regions and CDRs encodes an antibody that specifically binds to an antigen. In one embodiment, as discussed supra, one or more amino acid substitutions may be made within the framework regions, and, in certain embodiments, the amino acid substitutions improve binding of the antibody to its antigen. Techniques for humanization and optimization of antibodies are known in the art (see, e.g., Wu & An, 2003, Methods Mol. Biol., 207, 213-233; Wu, 2003, Methods Mol. Biol., 207, 197-212; Dall'Acqua et al. 2005, Methods, 36: 43-60 and U.S. Patent Publication No. 2006/0228350). Additionally, such methods may be used to make amino acid substitutions or deletions of one or more variable region cysteine residues participating in an intrachain disulfide bond to generate antibody molecules lacking one or more intrachain disulfide bonds. Other alterations to the polynucleotide are encompassed by the present invention and within the skill of the art.

The Fc region of antibodies identified from screening methods including, but not limited to, those described herein can be modified as described supra to generate an antibody incorporating a variant Fc region. It is further contemplated that the Fc variant proteins of the newly identified antibodies are useful for the prevention, management and treatment of a disease, disorder, infection, including but not limited to inflammatory diseases, autoimmune diseases, bone metabolism related disorders, angiogenic related disorders, infection, and cancer. Such antibodies are stabilized (e.g., will have reduced aggregation) by the formulations of the present invention.

### 6.5.2 Generating Fc Fusion Proteins

An Fc fusion protein combines an Fc region of an immunoglobulin or fragment thereof, with a fusion partner, which in general can be any protein, polypeptide, peptide, or small molecule. The role of the non-Fc part of the Fc fusion protein, i.e., the fusion partner, is often but not always to mediate target binding, and thus is functionally analogous to the variable regions of an antibody. Exemplary fusion partners are detailed supra (see, section entitled “Antigens, Fusion Partners and Antibodies”. A variety of linkers, defined and described herein, may be used to covalent link and Fc region to a fusion partner to generate an Fc fusion protein. Alternatively, Fc-fusion proteins may be produced by standard recombinant DNA techniques or by protein synthetic techniques, (e.g., by use of a peptide synthesizer). For example, a nucleic acid molecule encoding a fusion protein can be synthesized by conventional techniques including automated DNA synthesizers. Optionally, PCR amplification of gene fragments can be carried out using anchor primers which give rise to complementary overhangs between two consecutive gene fragments which can subsequently be annealed and reamplified to generate a chimeric gene sequence (see, e.g., Current Protocols in Molecular Biology, F. M. Ausubel et al., ed., John Wiley & Sons (Chichester, England, 1998); Molecular Cloning: A Laboratory Manual, 3rd Edition, J. Sambrook et al., ed., Cold Spring Harbor Laboratory Press (Cold Spring Harbor, N.Y., 2001)). Moreover, a nucleic acid encoding a fusion partner can be cloned into an expression vector containing the Fc region or a fragment thereof such that the fusion partner is linked in-frame to the constant domain or fragment thereof (e.g., Fc region).

Methods for fusing or conjugating polypeptides to the constant domains of antibodies are known in the art. See, e.g., U.S. Pat. Nos. 5,336,603; 5,622,929; 5,359,046; 5,349,053; 5,447,851; 5,723,125; 5,783,181; 5,908,626; 5,844,095; and 5,112,946; European Patent publications, EP 0 307 434; EP 0 367 166; EP 0 394 827; PCT publications WO 91/06570; WO 96/04388; WO 96/22024, WO 97/34631; and WO 99/04813; Ashkenazi et al., Proc. Natl. Acad. Sci. USA 88:10535-10539 (1991); Traunecker et al., Nature 331:84-86 (1988); Zheng et al., J. Immunol. 154:5590-5600 (1995); and Vil et al., Proc. Natl. Acad. Sci. USA 89:11337-11341 (1992).

Nucleotide sequences encoding protein molecules which may be used as fusion partners may be obtained from any information available to those of skill in the art (e.g., from Genbank, the literature, or by routine cloning), and the nucleotide sequence encoding an Fc region or a fragment thereof may be obtained from Genbank or the literature. The Fc region or a fragment thereof may be a naturally occurring domain or may be a variant Fc region including, but not limited to, those described herein. In the event that a naturally occurring Fc region is utilized, variants may be generated using methods known in the art including but not limited to those disclosed herein. The nucleotide sequence coding for a fusion protein can be inserted into an appropriate expression vector, i.e., a vector that contains the necessary elements for the transcription and translation of the inserted protein-coding sequence. A variety of host-vector systems may be utilized in the present invention to express the protein-coding sequence. These include but are not limited to mammalian cell systems infected with virus (e.g., vaccinia virus, adenovirus, etc.); insect cell systems infected with virus (e.g., baculovirus); microorganisms such as yeast containing yeast vectors; or bacteria transformed with bacteriophage, DNA, plasmid DNA, or cosmic DNA. The expression elements of vectors vary in their strengths and specificities. Depending on the host-vector system utilized, any one of a number of suitable transcription and translation elements may be used.

### 6.5.3 Recombinant Expression of Fc Variant Proteins

Recombinant expression of an Fc variant protein, derivative, analog or fragment thereof, (e.g., an antibody or Fc fusion protein), requires construction of an expression vector containing a polynucleotide that encodes the Fc variant protein. Once a polynucleotide encoding an Fc variant protein has been obtained, the vector for the production of the Fc variant protein may be produced by recombinant DNA technology using techniques well known in the art. Thus, methods for preparing a protein by expressing a polynucleotide containing a variant Fc region encoding nucleotide sequence are described herein. Methods that are well known to those skilled in the art can be used to construct expression vectors containing Fc variant protein coding sequences and appropriate transcriptional and translational control signals. These methods include, for example, in vitro recombinant DNA techniques, synthetic techniques, and in vivo genetic recombination. The invention, thus, provides replicable vectors comprising a nucleotide sequence encoding an Fc variant protein operably linked to a promoter. Such vectors may include the nucleotide sequence encoding the constant region of the antibody molecule (see, e.g., International Publication No. WO 86/05807; International Publication No. WO 89/01036; and U.S. Pat. No. 5,122,464) and the variable domain of the antibody, or a polypeptide for generating an Fc fusion protein may be cloned into such a vector for expression of the full length antibody chain (e.g. heavy or light chain), or complete Fc fusion protein comprising a fusion of a non-antibody derived polypeptide and a variant Fc region.

The expression vector is transferred to a host cell by conventional techniques and the transfected cells are then cultured by conventional techniques to produce an Fc variant protein. Thus, the invention includes host cells containing a polynucleotide encoding an Fc variant protein operably linked to a heterologous promoter. In specific embodiments for the expression of Fc variant proteins comprising double-chained antibodies, vectors encoding both the heavy and light chains may be co-expressed in the host cell for expression of the entire immunoglobulin molecule, as detailed below.

A variety of host-expression vector systems may be utilized to express the Fc variant proteins (e.g., antibody or Fc fusion protein) (see, e.g., U.S. Pat. No. 5,807,715). Such host-expression systems represent vehicles by which the coding sequences of interest may be produced and subsequently purified, but also represent cells which may, when transformed or transfected with the appropriate nucleotide coding sequences, express an Fc variant protein in situ. These include but are not limited to microorganisms such as bacteria (e.g., E. coli and B. subtilis) transformed with recombinant bacteriophage DNA, plasmid DNA or cosmid DNA expression vectors containing Fc variant protein coding sequences; yeast (e.g., Saccharomyces Pichia) transformed with recombinant yeast expression vectors containing Fc variant protein coding sequences; insect cell systems infected with recombinant virus expression vectors (e.g., baculovirus) containing Fc variant protein coding sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing Fc variant protein coding sequences; or mammalian cell systems (e.g., COS, CHO, BHK, 293, NS0, and 3T3 cells) harboring recombinant expression constructs containing promoters derived from the genome of mammalian cells (e.g., metallothionein promoter) or from mammalian viruses (e.g., the adenovirus late promoter; the vaccinia virus 7.5K promoter). In certain embodiments, bacterial cells such as Escherichia coli, or eukaryotic cells, are used for the expression of an Fc variant protein which is a recombinant antibody or an Fc fusion protein. For example, mammalian cells such as Chinese hamster ovary cells (CHO), in conjunction with a vector such as the major intermediate early gene promoter element from human cytomegalovirus are an effective expression system for antibodies (Foecking et al., 1986, Gene 45:101; and Cockett et al., 1990, Bio/Technology 8:2). In a specific embodiment, the expression of nucleotide sequences encoding an Fc variant protein is regulated by a constitutive promoter, inducible promoter or tissue specific promoter.

In bacterial systems, a number of expression vectors may be advantageously selected depending upon the use intended for the Fc variant protein (e.g., antibody or Fc fusion protein) being expressed. For example, when a large quantity of such a protein is to be produced, for the generation of formulations of an Fc variant protein for pharmaceutical use, vectors that direct the expression of high levels of fusion protein products that are readily purified may be desirable. Such vectors include, but are not limited to, the E. coli expression vector pUR278 (Ruther et al., 1983, EMBO 12:1791), in which the Fc variant protein coding sequence may be ligated individually into the vector in frame with the lac Z coding region so that a lac Z-fusion protein is produced; pIN vectors (Inouye & Inouye, 1985, Nucleic Acids Res. 13:3101-3109; Van Heeke & Schuster, 1989, J. Biol. Chem. 24:5503-5509); and the like. pGEX vectors may also be used to express foreign polypeptides as fusion proteins with glutathione 5-transferase (GST). In general, such fusion proteins are soluble and can easily be purified from lysed cells by adsorption and binding to matrix glutathione agarose beads followed by elution in the presence of free glutathione. The pGEX vectors are designed to include thrombin or factor Xa protease cleavage sites so that the cloned target gene product can be released from the GST moiety.

In an insect system, Autographa californica nuclear polyhedrosis virus (AcNPV) is used as a vector to express foreign genes. The virus grows in Spodoptera frugiperda cells. The Fc variant protein coding sequence may be cloned individually into non-essential regions (for example the polyhedrin gene) of the virus and placed under control of an AcNPV promoter (for example the polyhedrin promoter).

In mammalian host cells, a number of viral-based expression systems may be utilized. In cases where an adenovirus is used as an expression vector, the Fc variant protein coding sequence of interest may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing the Fc variant protein in infected hosts (e.g., see Logan & Shenk, 1984, Proc. Natl. Acad. Sci. USA 8 1:355-359). Specific initiation signals may also be required for efficient translation of inserted antibody coding sequences. These signals include the ATG initiation codon and adjacent sequences. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see, e.g., Bittner et al., 1987, Methods in Enzymol. 153:516-544).

The expression of an Fc variant protein may be controlled by any promoter or enhancer element known in the art. Promoters which may be used to control the expression of the gene encoding an Fc variant protein include, but are not limited to, the SV40 early promoter region (Bemoist and Chambon, 1981, Nature 290:304-310), the promoter contained in the 3′ long terminal repeat of Rous sarcoma virus (Yamamoto, et al., 1980, Cell 22:787-797), the herpes thymidine kinase promoter (Wagner et al., 1981, Proc. Natl. Acad. Sci. U.S.A. 78:1441-1445), the regulatory sequences of the metallothionein gene (Brinster et al., 1982, Nature 296:39-42), the tetracycline (Tet) promoter (Gossen et al., 1995, Proc. Nat. Acad. Sci. USA 89:5547-5551); prokaryotic expression vectors such as the β-lactamase promoter (VIIIa-Kamaroff et al., 1978, Proc. Natl. Acad. Sci. U.S.A. 75:3727-3731), or the tac promoter (DeBoer et al., 1983, Proc. Natl. Acad. Sci. U.S.A. 80:21-25; see also “Useful proteins from recombinant bacteria” in Scientific American, 1980, 242:74-94); plant expression vectors comprising the nopaline synthetase promoter region (Herrera-Estrella et al., Nature 303:209-213) or the cauliflower mosaic virus 35S RNA promoter (Gardner et al., 1981, Nucl. Acids Res. 9:2871), and the promoter of the photosynthetic enzyme ribulose biphosphate carboxylase (Herrera-Estrella et al., 1984, Nature 310:115-120); promoter elements from yeast or other fungi such as the Gal 4 promoter, the ADC (alcohol dehydrogenase) promoter, PGK (phosphoglycerol kinase) promoter, alkaline phosphatase promoter, and the following animal transcriptional control regions, which exhibit tissue specificity and have been utilized in transgenic animals: elastase I gene control region which is active in pancreatic acinar cells (Swift et al., 1984, Cell 38:639-646; Ornitz et al., 1986, Cold Spring Harbor Symp. Quant. Biol. 50:399-409; MacDonald, 1987, Hepatology 7:425-515); insulin gene control region which is active in pancreatic beta cells (Hanahan, 1985, Nature 315:115-122), immunoglobulin gene control region which is active in lymphoid cells (Grosschedl et al., 1984, Cell 38:647-658; Adames et al., 1985, Nature 318:533-538; Alexander et al., 1987, Mol. Cell. Biol. 7:1436-1444), mouse mammary tumor virus control region which is active in testicular, breast, lymphoid and mast cells (Leder et al., 1986, Cell 45:485-495), albumin gene control region which is active in liver (Pinkert et al., 1987, Genes and Devel. 1:268-276), alpha-fetoprotein gene control region which is active in liver (Krumlauf et al., 1985, Mol. Cell. Biol. 5:1639-1648; Hammer et al., 1987, Science 235:53-58; alpha 1-antitrypsin gene control region which is active in the liver (Kelsey et al., 1987, Genes and Devel. 1: 161-171), beta-globin gene control region which is active in myeloid cells (Mogram et al., 1985, Nature 315:338-340; Kollias et al., 1986, Cell 46:89-94; myelin basic protein gene control region which is active in oligodendrocyte cells in the brain (Readhead et al., 1987, Cell 48:703-712); myosin light chain-2 gene control region which is active in skeletal muscle (Sani, 1985, Nature 314:283-286); neuronal-specific enolase (NSE) which is active in neuronal cells (Morelli et al., 1999, Gen. Virol. 80:571-83); brain-derived neurotrophic factor (BDNF) gene control region which is active in neuronal cells (Tabuchi et al., 1998, Biochem. Biophysic. Res. Com. 253:818-823); glial fibrillary acidic protein (GFAP) promoter which is active in astrocytes (Gomes et al., 1999, Braz J Med Biol Res 32(5): 619-631; Morelli et al., 1999, Gen. Virol. 80:571-83) and gonadotropic releasing hormone gene control region which is active in the hypothalamus (Mason et al., 1986, Science 234:1372-1378).

Expression vectors containing inserts of a gene encoding an Fc variant protein can be identified by three general approaches: (a) nucleic acid hybridization, (b) presence or absence of “marker” gene functions, and (c) expression of inserted sequences. In the first approach, the presence of a gene encoding a peptide, polypeptide, protein or a fusion protein in an expression vector can be detected by nucleic acid hybridization using probes comprising sequences that are homologous to an inserted gene encoding the peptide, polypeptide, protein or the fusion protein, respectively. In the second approach, the recombinant vector/host system can be identified and selected based upon the presence or absence of certain “marker” gene functions (e.g., thymidine kinase activity, resistance to antibiotics, transformation phenotype, occlusion body formation in baculovirus, etc.) caused by the insertion of a nucleotide sequence encoding an antibody or fusion protein in the vector. For example, if the nucleotide sequence encoding the Fc variant protein is inserted within the marker gene sequence of the vector, recombinants containing the gene encoding the antibody or fusion protein insert can be identified by the absence of the marker gene function. In the third approach, recombinant expression vectors can be identified by assaying for the gene product (e.g., antibody or Fc fusion protein) expressed by the recombinant. Such assays can be based, for example, on the physical or functional properties of the fusion protein in in vitro assay systems, e.g., binding with anti-bioactive molecule antibody.

In addition, a host cell strain may be chosen which modulates the expression of the inserted sequences, or modifies and processes the gene product in the specific fashion desired. Expression from certain promoters can be elevated in the presence of certain inducers; thus, expression of the genetically engineered fusion protein may be controlled. Furthermore, different host cells have characteristic and specific mechanisms for the translational and post-translational processing and modification (e.g., glycosylation, phosphorylation of proteins). Appropriate cell lines or host systems can be chosen to ensure the desired modification and processing of the foreign protein expressed. For example, expression in a bacterial system will produce an unglycosylated product and expression in yeast will produce a glycosylated product. Eukaryotic host cells that possess the cellular machinery for proper processing of the primary transcript (e.g., glycosylation, and phosphorylation) of the gene product may be used. Such mammalian host cells include, but are not limited to, CHO, VERY, BHK, Hela, COS, MDCK, 293, 3T3, WI38, NSO, and in particular, neuronal cell lines such as, for example, SK-N-AS, SK-N-FI, SK-N-DZ human neuroblastomas (Sugimoto et al., 1984, J. Natl. Cancer Inst. 73: 51-57), SK-N-SH human neuroblastoma (Biochim. Biophys. Acta, 1982, 704: 450-460), Daoy human cerebellar medulloblastoma (He et al., 1992, Cancer Res. 52: 1144-1148) DBTRG-05MG glioblastoma cells (Kruse et al., 1992, In Vitro Cell. Dev. Biol. 28A: 609-614), IMR-32 human neuroblastoma (Cancer Res., 1970, 30: 2110-2118), 1321N1 human astrocytoma (Proc. Natl. Acad. Sci. USA, 1977, 74: 4816), MOG-G-CCM human astrocytoma (Br. J. Cancer, 1984, 49: 269), U87MG human glioblastoma-astrocytoma (Acta Pathol. Microbiol. Scand., 1968, 74: 465-486), A172 human glioblastoma (Olopade et al., 1992, Cancer Res. 52: 2523-2529), C6 rat glioma cells (Benda et al., 1968, Science 161: 370-371), Neuro-2a mouse neuroblastoma (Proc. Natl. Acad. Sci. USA, 1970, 65: 129-136), NB41A3 mouse neuroblastoma (Proc. Natl. Acad. Sci. USA, 1962, 48: 1184-1190), SCP sheep choroid plexus (Bolin et al., 1994, J. Virol. Methods 48: 211-221), G355-5, PG-4 Cat normal astrocyte (Haapala et al., 1985, J. Virol. 53: 827-833), Mpf ferret brain (Trowbridge et al., 1982, In Vitro 18: 952-960), and normal cell lines such as, for example, CTX TNA2 rat normal cortex brain (Radany et al., 1992, Proc. Natl. Acad. Sci. USA 89: 6467-6471) such as, for example, CRL7030 and Hs578Bst. Furthermore, different vector/host expression systems may effect processing reactions to different extents.

For long-term, high-yield production of recombinant proteins, stable expression is often preferred. For example, cell lines which stably express an Fc variant protein may be engineered. Rather than using expression vectors that contain viral origins of replication, host cells can be transformed with DNA controlled by appropriate expression control elements (e.g., promoter, enhancer, sequences, transcription terminators, polyadenylation sites, etc.), and a selectable marker. Following the introduction of the foreign DNA, engineered cells may be allowed to grow for 1-2 days in an enriched medium, and then are switched to a selective medium. The selectable marker in the recombinant plasmid confers resistance to the selection and allows cells to stably integrate the plasmid into their chromosomes and grow to form foci that in turn can be cloned and expanded into cell lines. This method may advantageously be used to engineer cell lines that express an Fc variant protein that specifically binds to an Antigen. Such engineered cell lines may be particularly useful in screening and evaluation of compounds that affect the activity of an Fc protein that specifically binds to an antigen.

A number of selection systems may be used, including but not limited to the herpes simplex virus thymidine kinase (Wigler et al., 1977, Cell 11:223), hypoxanthine-guanine phosphoribosyltransferase (Szybalska & Szybalski, 1962, Proc. Natl. Acad. Sci. USA 48:2026), and adenine phosphoribosyltransferase (Lowy et al., 1980, Cell 22:817) genes can be employed in tk−, hgprt− or aprt− cells, respectively. Also, antimetabolite resistance can be used as the basis of selection for dhfr, which confers resistance to methotrexate (Wigler et al., 1980, Natl. Acad. Sci. USA 77:3567; O'Hare et al., 1981, Proc. Natl. Acad. Sci. USA 78:1527); gpt, which confers resistance to mycophenolic acid (Mulligan & Berg, 1981, Proc. Natl. Acad. Sci. USA 78:2072); neo, which confers resistance to the aminoglycoside G-418 (Colberre-Garapin et al., 1981, J. Mol. Biol. 150:1); and hygro, which confers resistance to hygromycin (Santerre et al., 1984, Gene 30:147) genes.

The expression levels of an Fc variant protein can be increased by vector amplification (for a review, see Bebbington and Hentschel, The use of vectors based on gene amplification for the expression of cloned genes in mammalian cells in DNA cloning, Vol. 3. (Academic Press, New York, 1987)). For example, when a marker in the vector system expressing an antibody or Fc fusion protein is amplifiable, increase in the level of inhibitor present in culture of host cell will increase the number of copies of the marker gene. Since the amplified region is associated with the antibody gene, production of the antibody or fusion protein will also increase (Crouse et al., 1983, Mol. Cell. Biol. 3:257).

The host cell may be co-transfected with two expression vectors of the invention. For example, the first vector encoding a heavy chain derived polypeptide and the second vector encoding a light chain derived polypeptide. The two vectors may contain identical selectable markers, which enable equal expression of heavy and light chain polypeptides. Alternatively, a single vector may be used which encodes, and is capable of expressing, a fusion protein or both heavy and light chain polypeptides. The coding sequences for the fusion protein or heavy and light chains may comprise cDNA or genomic DNA.

### 6.5.4 Purification of Fc Variant Proteins

Once an Fc protein has been produced by recombinant expression, it may be purified by any method known in the art for purification of a protein, for example, by chromatography (e.g., ion exchange, affinity, particularly by affinity for the specific antigen after Protein A, and sizing column chromatography), centrifugation, differential solubility, or by any other standard technique for the purification of proteins.

An “isolated” or “purified” Fc variant protein is substantially free of cellular material or other contaminating proteins from the cell or tissue source from which the protein is derived, or substantially free of chemical precursors or other chemicals when chemically synthesized. The language “substantially free of cellular material” includes preparations of an Fc variant protein in which the Fc variant protein is separated from cellular components of the cells from which it is isolated or recombinantly produced. Thus, an Fc variant protein that is substantially free of cellular material includes preparations of Fc variant protein having less than about 30%, 20%, 10%, or 5% (by dry weight) of heterologous protein (also referred to herein as a “contaminating protein”). When the Fc variant protein is recombinantly produced, it is also preferably substantially free of culture medium, i.e., culture medium represents less than about 20%, 10%, or 5% of the volume of the protein preparation. When the Fc variant protein is produced by chemical synthesis, it is preferably substantially free of chemical precursors or other chemicals, i.e., it is separated from chemical precursors or other chemicals which are involved in the synthesis of the protein. Accordingly such preparations of the Fc variant protein have less than about 30%, 20%, 10%, 5% (by dry weight) of chemical precursors or compounds other than the antibody of interest. In certain embodiments, Fc variant proteins are isolated or purified prior to or concurrently with being formulated according to the present invention.

Generally, the expression of an Fc variant protein is first confirmed, for example, by gel electrophoresis using SDS-PAGE reducing or non-reducing protein gel analysis, or any other techniques known in the art. ELISA can also be used to detect both the expression of an Fc variant protein and the quantity of that Fc variant protein present. The modified Fc-fusion proteins described herein may be produced intracellularly, in the periplasmic space, or directly secreted into the medium. In one embodiment, the Fc variant proteins are secreted into culture media. The media of the host cell culture producing Fc variant proteins are collected and cell debris is spun down by centrifugation. The supernatants are collected and subjected to the protein purification methods. Methods of preparation and purification of monoclonal and polyclonal antibodies are known in the art and e.g., are described in Harlow and Lane, Antibodies: A Laboratory Manual (New York: Cold Spring Harbor Laboratory Press, 1988). It may be desirable to concentrate the purified Fc variant proteins. Methods to concentrate proteins are well known in the art and include using a semipermeable membrane with an appropriate molecular weight (MW) cutoff (e.g., 30 kD cutoff for whole antibody molecules). Numerous methods may be utilized to formulate the purified Fc variant proteins into the formulations of the invention. For example, difiltration, may be utilized for buffer exchange, this method may be used for both concentration and buffer exchange. It will be understood by one skilled in the art that Fc variant proteins may be first formulated into a base buffer comprising some but not all the components of a formulation of the invention, for example by difiltration, and afterwards the remaining components of the formulation are added to generate a final formulation comprising all the desired components at the preferred concentrations. Generally, the minimum acceptable purity of an Fc variant protein for use in pharmaceutical formulation will be 90%, with 95% preferred, 98% more preferred and 99% or higher the most preferred.

### 6.6 Methods of Using

The present invention encompasses administering the formulations of the invention comprising one or more Fc variant protein (e.g., antibodies comprising a variant Fc region) to an animal, preferably a mammal, and most preferably a human, for preventing, treating, or ameliorating one or more symptoms associated with a disease, disorder, or infection. Fc variant proteins are particularly useful for the treatment or prevention of a disease or disorder where an altered efficacy of effector cell function (e.g., ADCC, CDC) is desired. The formulations of the invention comprising Fc variant protein are particularly useful for the treatment or prevention of primary or metastatic neoplastic disease (i.e., cancer), and infectious diseases. Formulations of the invention comprising pharmaceutically acceptable components maybe generated as described herein. As detailed below, the formulations of the invention can be used in methods of treating or preventing cancer (particularly in passive immunotherapy), autoimmune disease, inflammatory disorders or infectious diseases.

The formulations of the invention may also be advantageously utilized in combination with other therapeutic agents known in the art for the treatment or prevention of a cancer, autoimmune disease, inflammatory disorders or infectious diseases. In a specific embodiment, formulations of the invention may be used in combination with monoclonal or chimeric antibodies, lymphokines, or hematopoietic growth factors (such as, e.g., IL-2, IL-3 and IL-7), which, for example, serve to increase the number or activity of effector cells which interact with the molecules and, increase immune response. The formulations of the invention may also be advantageously utilized in combination with one or more drugs used to treat a disease, disorder, or infection such as, for example anti-cancer agents, anti-inflammatory agents or anti-viral agents.

The invention further encompasses administering the formulations of the invention in combination with other therapies known to those skilled in the art for the treatment or prevention of cancer, including but not limited to, current standard and experimental chemotherapies, hormonal therapies, biological therapies, immunotherapies, radiation therapies, or surgery. In some embodiments, the formulations of the invention may be administered in combination with a therapeutically or prophylactically effective amount of one or more anti-cancer agents, therapeutic antibodies or other agents known to those skilled in the art for the treatment and/or prevention of cancer. Examples of dosing regimes and therapies which can be used in combination with the formulations of the invention are well known in the art and have been described in detail elsewhere (see for example, PCT publications WO 02/070007 and WO 03/075957).

Cancers and related disorders that can be treated or prevented by methods and compositions of the present invention include, but are not limited to, the following: Leukemias, lymphomas, multiple myelomas, bone and connective tissue sarcomas, brain tumors, breast cancer, adrenal cancer, thyroid cancer, pancreatic cancer, pituitary cancers, eye cancers, vaginal cancers, vulvar cancer, cervical cancers, uterine cancers, ovarian cancers, esophageal cancers, stomach cancers, colon cancers, rectal cancers, liver cancers, gallbladder cancers, cholangiocarcinomas, lung cancers, testicular cancers, prostate cancers, penal cancers; oral cancers, salivary gland cancers pharynx cancers, skin cancers, kidney cancers, bladder cancers (for a review of such disorders, see Fishman et al., 1985, Medicine, 2d Ed., J.B. Lippincott Co., Philadelphia and Murphy et al., 1997, Informed Decisions: The Complete Book of Cancer Diagnosis, Treatment, and Recovery, Viking Penguin, Penguin Books U.S.A., Inc., United States of America).

In a specific embodiment, a formulation of the invention alone or in combination with other anti-cancer agents or treatments inhibits or reduces the growth of primary tumor or metastasis of cancerous cells by at least 99%, at least 95%, at least 90%, at least 85%, at least 80%, at least 75%, at least 70%, at least 60%, at least 50%, at least 45%, at least 40%, at least 45%, at least 35%, at least 30%, at least 25%, at least 20%, or at least 10% relative to the growth of primary tumor or metastasis in the absence of said formulation of the invention.

The present invention encompasses the use of one or more formulation of the invention for preventing, treating, or managing one or more symptoms associated with an inflammatory disorder in a subject.

The invention further encompasses administering the formulations of the invention in combination with a therapeutically or prophylactically effective amount of one or more anti-inflammatory agents. The invention also provides methods for preventing, treating, or managing one or more symptoms associated with an autoimmune disease further comprising, administering to said subject a formulation of the invention in combination with a therapeutically or prophylactically effective amount of one or more immunomodulatory agents. Examples of autoimmune disorders that may be treated by administering the formulations of the invention include, but are not limited to, alopecia greata, ankylosing spondylitis, antiphospholipid syndrome, autoimmune Addison's disease, autoimmune diseases of the adrenal gland, autoimmune hemolytic anemia, autoimmune hepatitis, autoimmune oophoritis and orchitis, autoimmune thrombocytopenia, Behcet's disease, bullous pemphigoid, cardiomyopathy, celiac sprue-dermatitis, chronic fatigue immune dysfunction syndrome (CFIDS), chronic inflammatory demyelinating polyneuropathy, Churg-Strauss syndrome, cicatrical pemphigoid, CREST syndrome, cold agglutinin disease, Crohn's disease, discoid lupus, essential mixed cryoglobulinemia, fibromyalgia-fibromyositis, glomerulonephritis, Graves' disease, Guillain-Barre, Hashimoto's thyroiditis, idiopathic pulmonary fibrosis, idiopathic thrombocytopenia purpura (ITP), IgA neuropathy, juvenile arthritis, lichen planus, lupus erthematosus, Meniere's disease, mixed connective tissue disease, multiple sclerosis, type 1 or immune-mediated diabetes mellitus, myasthenia gravis, pemphigus vulgaris, pernicious anemia, polyarteritis nodosa, polychrondritis, polyglandular syndromes, polymyalgia rheumatica, polymyositis and dermatomyositis, primary agammaglobulinemia, primary biliary cirrhosis, psoriasis, psoriatic arthritis, Raynauld's phenomenon, Reiter's syndrome, Rheumatoid arthritis, sarcoidosis, scleroderma, Sjogren's syndrome, stiff-man syndrome, systemic lupus erythematosus, lupus erythematosus, takayasu arteritis, temporal arteristis/giant cell arteritis, ulcerative colitis, uveitis, vasculitides such as dermatitis herpetiformis vasculitis, vitiligo, and Wegener's granulomatosis. Examples of inflammatory disorders include, but are not limited to, asthma, encephilitis, inflammatory bowel disease, chronic obstructive pulmonary disease (COPD), allergic disorders, septic shock, pulmonary fibrosis, undifferentitated spondyloarthropathy, undifferentiated arthropathy, arthritis, inflammatory osteolysis, and chronic inflammation resulting from chronic viral or bacteria infections. Some autoimmune disorders are associated with an inflammatory condition, thus, there is overlap between what is considered an autoimmune disorder and an inflammatory disorder. Therefore, some autoimmune disorders may also be characterized as inflammatory disorders. Examples of inflammatory disorders which can be prevented, treated or managed in accordance with the methods of the invention include, but are not limited to, asthma, encephilitis, inflammatory bowel disease, chronic obstructive pulmonary disease (COPD), allergic disorders, septic shock, pulmonary fibrosis, undifferentitated spondyloarthropathy, undifferentiated arthropathy, arthritis, inflammatory osteolysis, and chronic inflammation resulting from chronic viral or bacteria infections.

Formulation of the invention can also be used to reduce the inflammation experienced by animals, particularly mammals, with inflammatory disorders. In a specific embodiment, a formulation of the invention along or in combination with another anti-inflammatory agent or therapy reduces the inflammation in an animal by at least 99%, at least 95%, at least 90%, at least 85%, at least 80%, at least 75%, at least 70%, at least 60%, at least 50%, at least 45%, at least 40%, at least 45%, at least 35%, at least 30%, at least 25%, at least 20%, or at least 10% relative to the inflammation in an animal, which is not administered the formulation of the invention.

The invention also encompasses methods for treating or preventing an infectious disease in a subject comprising administering a therapeutically or prophylatically effective amount of a formulation of the invention. Infectious diseases that can be treated or prevented by the formulations of the invention are caused by infectious agents including but not limited to viruses, bacteria, fungi, protozae, and viruses.

Viral diseases that can be treated or prevented using the formulations of the invention in conjunction with the methods of the present invention include, but are not limited to, those caused by hepatitis type A, hepatitis type B, hepatitis type C, influenza, varicella, adenovirus, herpes simplex type I (HSV-T), herpes simplex type II (HSV-II), rinderpest, rhinovirus, echovirus, rotavirus, respiratory syncytial virus, papilloma virus, papova virus, cytomegalovirus, echinovirus, arbovirus, huntavirus, coxsackie virus, mumps virus, measles virus, rubella virus, polio virus, small pox, Epstein Barr virus, human immunodeficiency virus type I (HIV-I), human immunodeficiency virus type II (HIV-II), and agents of viral diseases such as viral miningitis, encephalitis, dengue or small pox.

Bacterial diseases that can be treated or prevented using the formulations of the invention in conjunction with the methods of the present invention, that are caused by bacteria include, but are not limited to, mycobacteria rickettsia, mycoplasma, neisseria, S. pneumonia, Borrelia burgdorferi (Lyme disease), Bacillus antracis (anthrax), tetanus, streptococcus, staphylococcus, mycobacterium, tetanus, pertissus, cholera, plague, diptheria, chlamydia, S. aureus and legionella. Protozoal diseases that can be treated or prevented using the molecules of the invention in conjunction with the methods of the present invention, that are caused by protozoa include, but are not limited to, leishmania, kokzidioa, trypanosoma or malaria. Parasitic diseases that can be treated or prevented using the formulations of the invention in conjunction with the methods of the present invention, that are caused by parasites include, but are not limited to, chlamydia and rickettsia.

In some embodiments, the formulations of the invention may be administered in combination with a therapeutically or prophylactically effective amount of one or additional therapeutic agents known to those skilled in the art for the treatment and/or prevention of an infectious disease. The invention contemplates the use of the molecules of the invention in combination with other molecules known to those skilled in the art for the treatment and or prevention of an infectious disease including, but not limited to, antibiotics, antifungal agents and anti-viral agents.

Accordingly, the present invention provides methods for preventing, treating, or ameliorating one or more symptoms associated with disease, disorder, or infection by administering to a subject an effective amount of a formulation of the invention. In a one aspect, the formulation comprises an Fc variant protein that is substantially purified (i.e., substantially free from substances that limit its effect or produce undesired side-effects). In a specific embodiment, the subject is an animal, such as a mammal including non-primates (e.g., cows, pigs, horses, cats, dogs, rats etc.) and primates (e.g., monkey such as, a cynomolgous monkey and a human). In a specific embodiment, the subject is a human. In yet another specific embodiment, the Fc variant protein is derived from the same species as the subject.

The invention provides methods for preventing, treating, or ameliorating one or more symptoms associated with a disease, disorder, or infection, said method comprising: (a) administering to a subject in need thereof a dose of a prophylactically or therapeutically effective amount of a formulation of the invention and (b) administering one or more subsequent doses of said formulation, to maintain a plasma concentration of the Fc variant protein at a desirable level (e.g., about 0.1 to about 100 μg/ml), which continuously binds to an antigen or target molecule. In a specific embodiment, the plasma concentration of the Fc variant protein is maintained at 10 μg/ml, 15 μg/ml, 20 μg/ml, 25 μg/ml, 30 μg/ml, 35 μg/ml, 40 μg/ml, 45 μg/ml or 50 μg/ml. In a specific embodiment, said effective amount of Fc variant protein to be administered is between at least 1 mg/kg and 8 mg/kg per dose. In another specific embodiment, said effective amount of Fc variant protein to be administered is between at least 4 mg/kg and 8 mg/kg per dose. In yet another specific embodiment, said effective amount of Fc variant protein to be administered is between 50 mg and 250 mg per dose. In still another specific embodiment, said effective amount of Fc variant protein to be administered is between 100 mg and 200 mg per dose.

The present invention also encompasses protocols for preventing, treating, or ameliorating one or more symptoms associated with a disease, disorder, or infection which a formulation of the invention is used in combination with a therapy (e.g., prophylactic or therapeutic agent) other than a formulation of the invention. The invention is based, in part, on the recognition that the components of a formulations the invention, specifically the Fc variant proteins, potentiate and synergize with, enhance the effectiveness of, improve the tolerance of, and/or reduce the side effects caused by, other therapies, including current standard and experimental therapies. The combination therapies of the invention have additive potency, an additive therapeutic effect or a synergistic effect. The combination therapies of the invention enable lower dosages of the therapy (e.g., prophylactic or therapeutic agents) utilized in conjunction with the formulations of the invention for preventing, treating, or ameliorating one or more symptoms associated with a disease, disorder, or infection and/or less frequent administration of such prophylactic or therapeutic agents to a subject with a disease disorder, to improve the quality of life of said subject and/or to achieve a prophylactic or therapeutic effect. Further, the combination therapies described herein can reduce or avoid unwanted or adverse side effects associated with the administration of current single agent therapies and/or existing combination therapies, which in turn improves patient compliance with the treatment protocol. Numerous molecules which can be utilized in combination with the formulations of the invention are well known in the art. See for example, PCT publications WO 02/070007; WO 03/075957 and U.S. Patent Publication 2005/064514.

The route of administration of the composition depends on the condition to be treated. For example, intravenous injection may be preferred for treatment of a systemic disorder such as a lymphatic cancer or a tumor which has metastasized. The dosage of the compositions to be administered can be determined by the skilled artisan without undue experimentation in conjunction with standard dose-response studies. Relevant circumstances to be considered in making those determinations include the condition or conditions to be treated, the choice of composition to be administered, the age, weight, and response of the individual patient, and the severity of the patient's symptoms. Depending on the condition, the composition can be administered orally, parenterally, intranasally, vaginally, rectally, lingually, sublingually, buccally, intrabuccally and/or transdermally to the patient.

Accordingly, the formulations of the invention may be designed for oral, lingual, sublingual, buccal and intrabuccal administration can be made without undue experimentation by means well known in the art, for example, with an inert diluent or with an edible carrier. The formulations of the invention may be enclosed in gelatin capsules or compressed into tablets. For the purpose of oral therapeutic administration, the formulations of the present invention may be incorporated with excipients and used in the form of tablets, troches, capsules, elixirs, suspensions, syrups, wafers, chewing gums, and the like.

Tablets, pills, capsules, troches and the like may also contain binders, recipients, disintegrating agent, lubricants, sweetening agents, and/or flavoring agents. Some examples of binders include microcrystalline cellulose, gum tragacanth and gelatin. Examples of excipients include starch and lactose. Some examples of disintegrating agents include alginic acid, cornstarch, and the like. Examples of lubricants include magnesium stearate and potassium stearate. An example of a glidant is colloidal silicon dioxide. Some examples of sweetening agents include sucrose, saccharin, and the like. Examples of flavoring agents include peppermint, methyl salicylate, orange flavoring, and the like. Materials used in preparing these various compositions should be pharmaceutically pure and non-toxic in the amounts used.

The formulations of the present invention can be administered parenterally, such as, for example, by intravenous, intramuscular, intrathecal and/or subcutaneous injection. Parenteral administration can be accomplished by incorporating the formulations of the present invention into a solution or suspension. Such solutions or suspensions may also include sterile diluents, such as water for injection, saline solution, fixed oils, polyethylene glycols, glycerine, propylene glycol and/or other synthetic solvents. Parenteral formulations may also include antibacterial agents, such as, for example, benzyl alcohol and/or methyl parabens, antioxidants, such as, for example, ascorbic acid and/or sodium bisulfite, and chelating agents, such as EDTA. Buffers, such as acetates, citrates and phosphates, and agents for the adjustment of tonicity, such as sodium chloride and dextrose, may also be added. The parenteral preparation can be enclosed in ampules, disposable syringes and/or multiple dose vials made of glass or plastic. Rectal administration includes administering the composition into the rectum and/or large intestine. This can be accomplished using suppositories and/or enemas. Suppository formulations can be made by methods known in the art. Transdermal administration includes percutaneous absorption of the composition through the skin. Transdermal formulations include patches, ointments, creams, gels, salves, and the like. The formulations of the present invention can be administered nasally to a patient. As used herein, nasally administering or nasal administration includes administering the compositions to the mucous membranes of the nasal passage and/or nasal cavity of the patient.

### 7. SPECIFIC EMBODIMENTS

1. A formulation comprising an Fc variant protein, a buffering agent at a concentration between about 1 mM to about 100 mM and further comprising one or more component selected from the group consisting of:
   (a) a carbohydrate excipient at a concentration between about 1% to about 20% weight to volume;
   (b) a cationic amino acid at a concentration between about 1 mM to about 400 mM;
   (c) an anion at a concentration between about 1 mM to about 200 mM; and
   (d) a polysorbate at a concentration between about 0.001% to about 0.1%, wherein, said formulation has a pH of about 5.5 to about 8.0.
2. The formulation of embodiment 1, comprising component (a), but not (b), (c) or (d).
3. The formulation of embodiment 1, comprising component (a) and (b), but not (c) or (d).
4. The formulation of embodiment 1, comprising component (a) and (c), but not (b) or (d).
5. The formulation of embodiment 1, comprising component (a) and (d), but not (b) or (c).
6. The formulation of embodiment 1, comprising component (b) and (c), but not (a) or (d).
7. The formulation of embodiment 1, comprising component (b) and (d), but not (a) or (c).
8. The formulation of embodiment 1, comprising component (c) and (d), but not (a) or (b).
9. The formulation of embodiment 1, comprising component (b) and (c) and (d) but not (a).
10. The formulation of embodiment 1, comprising component (a) and (c) and (d) but not (b).
11. The formulation of embodiment 1, comprising component (a) and (b) and (d) but not (c).
12. The formulation of embodiment 1, comprising component (a) and (b) and (c) but not (d).
13. The formulation of embodiment 1, comprising component (a) and (b) and (c) and (d).
14. The formulation of any of the preceding embodiments, wherein the buffer is an anion.
15. The formulation of any of the preceding embodiments, wherein the Fc variant protein has at least 10% less aggregation when compared to the aggregation when the same Fc variant protein is formulated in 10 mM Histidine pH 6.0.
16. The formulation of any of the preceding embodiments, wherein the Fc variant protein has no more than about 2% aggregate, relative to total Fc variant protein at the temperature range of 2° C. to 8° C. for at least 90 days, as assessed by sized exclusion chromatograph (SEC).
17. The formulation of any of the preceding embodiment, wherein the formulation does not comprise cysteine as an excipient and/or additive.
18. The formulation of any of the preceding embodiments, wherein the Fc variant protein concentration is between about 10 mg/mL and about 200 mg/mL.
19. The formulation of any of the preceding embodiments, wherein the Fc variant protein is an antibody.
20. The formulation of any of the embodiments 1 to 18, wherein the Fc variant protein is an Fc fusion protein.
21. The formulation of embodiment 19 or 20, wherein the Fc variant protein binds the same antigen as a clinical product or candidate selected from the group consisting of: rituximab, zanolimumab, hA20, AME-133, HumaLYM™, trastuzumab, pertuzumab, cetuximab, IMC-3G3, panitumumab, zalutumumab, nimotuzumab, matuzumab, ch806, KSB-102, MR1-1, SC100, SC101, SC103, alemtuzumab, muromonab-CD3, OKT4A, ibritumomab, gemtuzumab, alefacept, abciximab, basiliximab, palivizumab, motavizumab, infliximab, adalimumab, CDP-571, etanercept, ABX-CBL, ABX-IL8, ABX-MA1 pemtumomab, Therex, AS1405, natalizumab, HuBC-1, natalizumab, IDEC-131, VLA-1; CAT-152; J695, CAT-192, CAT-213, BR3-Fc, LymphoStat-B™, TRAIL-R1mAb, bevacizumab, ranibizumab, omalizumab, efalizumab, MLN-02, zanolimumab, HuMax-IL 15, HuMax-Inflam, HuMax-Cancer, HuMax-Lymphoma, HuMax-TAC, clenoliximab, lumiliximab, BEC2, IMC-1C11, D 101, labetuzumab, arcitumomab, epratuzumab, tacatuzumab, MyelomaCide™, LkoCide™, ProstaCide™, ipilimumab, MDX-060, MDX-070, MDX-018, MDX-1106, MDX-1103, MDX-1333, MDX-214, MDX-1100, MDX-CD4, MDX-1388, MDX-066, MDX-1307, HGS-TR2J, FG-3019, BMS-66513, SGN-30, SGN-40, tocilizumab, CS-1008, IDM-1, golimumab, CNTO 1275, CNTO 95, CNTO 328, mepolizumab, MOR101, MOR102, MOR201, visilizumab, HuZAF™, volocixmab, ING-1, MLN2201, daclizumab, HCD122, CDP860, PRO542, C14, oregovomab, edrecolomab, etaracizumab, siplizumab, lintuzumab, Hu1D10, Lym-1, efalizumab, ICM3, galiximab, eculizumab, pexelizumab, LDP-01, huA33, WX-G250, sibrotuzumab, Chimeric KW-2871, hu3S193, huLK26; bivatuzumab, ch14.18, 3F8, BC8, huHMFG1, MORAb-003, MORAb-004, MORAb-009, denosumab, PRO-140, 1D09C3, huMikbeta-1, NI-0401, NI-501, cantuzumab, HuN901, 8H9, chTNT-1/B, bavituximab, huJ591, HeFi-1, Pentacea™, abagovomab, tositumomab, 105AD7, GMA161 and GMA321.
22. The formulation of embodiment 19, wherein the Fc variant protein competes for binding to the same antigen as a clinical product or candidate selected from the group consisting of: rituximab, zanolimumab, hA20, AME-133, HumaLYM™, trastuzumab, pertuzumab, cetuximab, IMC-3G3, panitumumab, zalutumumab, nimotuzumab, matuzumab, ch806, KSB-102, MR1-1, SC100, SC101, SC103, alemtuzumab, muromonab-CD3, OKT4A, ibritumomab, gemtuzumab, alefacept, abciximab, basiliximab, palivizumab, motavizumab, infliximab, adalimumab, CDP-571, etanercept, ABX-CBL, ABX-IL8, ABX-MA1 pemtumomab, Therex, AS1405, natalizumab, HuBC-1, natalizumab, IDEC-131, VLA-1; CAT-152; J695, CAT-192, CAT-213, BR3-Fc, LymphoStat-B™, TRAIL-R1mAb, bevacizumab, ranibizumab, omalizumab, efalizumab, MLN-02, zanolimumab, HuMax-IL 15, HuMax-Inflam, HuMax-Cancer, HuMax-Lymphoma, HuMax-TAC, clenoliximab, lumiliximab, BEC2, IMC-1C11, DC101, labetuzumab, arcitumomab, epratuzumab, tacatuzumab, MyelomaCide™, LkoCide™, ProstaCide™, ipilimumab, MDX-060, MDX-070, MDX-018, MDX-1106, MDX-1103, MDX-1333, MDX-214, MDX-1100, MDX-CD4, MDX-1388, MDX-066, MDX-1307, HGS-TR2J, FG-3019, BMS-66513, SGN-30, SGN-40, tocilizumab, CS-1008, IDM-1, golimumab, CNTO 1275, CNTO 95, CNTO 328, mepolizumab, MOR101, MOR102, MOR201, visilizumab, HuZAF™, volocixmab, ING-1, MLN2201, daclizumab, HCD122, CDP860, PRO542, C14, oregovomab, edrecolomab, etaracizumab, siplizumab, lintuzumab, Hu1D10, Lym-1, efalizumab, ICM3, galiximab, eculizumab, pexelizumab, LDP-01, huA33, WX-G250, sibrotuzumab, Chimeric KW-2871, hu3S193, huLK26; bivatuzumab, ch14.18, 3F8, BC8, huHMFG1, MORAb-003, MORAb-004, MORAb-009, denosumab, PRO-140, 1D09C3, huMikbeta-1, NI-0401, NI-501, cantuzumab, HuN901, 8H9, chTNT-1/B, bavituximab, huJ591, HeFi-1, Pentacea™, abagovomab, tositumomab, 105AD7, GMA161 and GMA321.
23. The formulation of embodiment 19, wherein the Fc variant proteincomprises at least one CDR from a clinical product or candidate selected from the group consisting of: rituximab, zanolimumab, hA20, AME-133, HumaLYM™, trastuzumab, pertuzumab, cetuximab, IMC-3G3, panitumumab, zalutumumab, nimotuzumab, matuzumab, ch806, KSB-102, MR1-1, SC100, SC101, SC103, alemtuzumab, muromonab-CD3, OKT4A, ibritumomab, gemtuzumab, alefacept, abciximab, basiliximab, palivizumab, motavizumab, infliximab, adalimumab, CDP-571, ABX-CBL, ABX-IL8, ABX-MA1 pemtumomab, Therex, AS1405, natalizumab, HuBC-1, natalizumab, IDEC-131, VLA-1; CAT-152; J695, CAT-192, CAT-213, LymphoStat-B™, TRAIL-R1mAb, bevacizumab, ranibizumab, omalizumab, efalizumab, MLN-02, zanolimumab, HuMax-IL 15, HuMax-Inflam, HuMax-Cancer, HuMax-Lymphoma, HuMax-TAC, clenoliximab, lumiliximab, BEC2, IMC-1C11, DC101, labetuzumab, arcitumomab, epratuzumab, tacatuzumab, MyelomaCide™, LkoCide™, ProstaCide™, ipilimumab, MDX-060, MDX-070, MDX-018, MDX-1106, MDX-1103, MDX-1333, MDX-214, MDX-1100, MDX-CD4, MDX-1388, MDX-066, MDX-1307, HGS-TR2J, FG-3019, BMS-66513, SGN-30, SGN-40, tocilizumab, CS-1008, IDM-1, golimumab, CNTO 1275, CNTO 95, CNTO 328, mepolizumab, MOR101, MOR102, MOR201, visilizumab, HuZAF™, volocixmab, ING-1, MLN2201, daclizumab, HCD122, CDP860, PRO542, C14, oregovomab, edrecolomab, etaracizumab, siplizumab, lintuzumab, Hu1D10, Lym-1, efalizumab, ICM3, galiximab, eculizumab, pexelizumab, LDP-01, huA33, WX-G250, sibrotuzumab, Chimeric KW-2871, hu3S193, huLK26; bivatuzumab, ch14.18, 3F8, BC8, huHMFG1, MORAb-003, MORAb-004, MORAb-009, denosumab, PRO-140, 1D09C3, huMikbeta-1, NI-0401, NI-501, cantuzumab, HuN901, 8H9, chTNT-1/B, bavituximab, huJ591, HeFi-1, Pentacea™, abagovomab, tositumomab, 105AD7, GMA161 and GMA321.
24. The formulation of any of the preceding embodiments, wherein the Fc variant protein comprises at least one of the amino acids sequences selected from the group consisting of: SEQ ID NOS: 2, 4, 6 and 8-20.
25. The formulation of any of embodiments 1 to 13, wherein the concentration of buffering agent is between about 10 mM to about 50 mM.
26. The formulation of any of embodiments 1 to 13, wherein the buffering agent is between about 50 mM to about 100 mM.
27. The formulation of any of embodiments 1 to 13, wherein the buffering agent is selected from the group consisting of histidine, phosphate and citrate.
28. The formulation of embodiment 27, wherein the buffering agent is histidine.
29. The formulation of embodiment 27, wherein the buffering agent is phosphate.
30. The formulation of embodiment 27, wherein the buffering agent is citrate.
31. The formulation of any of embodiments 1 to 13, wherein the concentration of carbohydrate excipient is between about 5% to about 20% weight to volume.
32. The formulation of any of embodiments 1 to 13, wherein the carbohydrate excipient is selected from the group consisting of trehalose, sucrose, mannitol, maltose, and raffinose.
33. The formulation of embodiment 32, wherein the carbohydrate excipient is trehalose.
34. The formulation of embodiment 32, wherein the carbohydrate excipient is sucrose.
35. The formulation of any of embodiments 1, 3, 6, 7, 9, 11, 12 or 13, wherein the concentration of cationic amino acid is between about 35 mM to about 200 mM.
36. The formulation of any of embodiments 1, 3, 6, 7, 9, 11, 12 or 13, wherein the cationic amino acid is selected from the group consisting of lysine, arginine and histidine.
37. The formulation of embodiment 36, wherein the cationic amino acid is lysine.
38. The formulation of embodiment 36, wherein the cationic amino acid is arginine.
39. The formulation of embodiment 36, wherein the cationic amino acid is histidine.
40. The formulation of any of embodiments 1, 3, 6, 7, 9, 11, 12 or 13, wherein the concentration of anion is between about 10 mM to about 50 mM.
41. The formulation of any of embodiments 1, 3, 6, 7, 9, 11, 12 or 13, wherein the concentration of anion is between about 50 mM to about 100 mM.
42. The formulation of any of embodiments 1, 3, 6, 7, 9, 11, 12 or 13, wherein the concentration of anion is between about 100 mM to about 200 mM.
43. The formulation of any of embodiments 1, 3, 6, 7, 9, 11, 12 or 13, wherein the anion is selected from the group consisting of citrate, succinate and phosphate.
44. The formulation of embodiment 43, wherein the anion is citrate.
45. The formulation of embodiment 43, wherein the anion is succinate.
46. The formulation of embodiment 43, wherein the anion is phosphate.
47. The formulation of embodiment 43, wherein the anion is citrate and the buffer is citrate, and wherein the total concentration of citrate is between about 100 mM and 300 mM.
48. The formulation of embodiment 47, wherein the total concentration of citrate is about 100 mM.
49. The formulation of embodiment 47, wherein the total concentration of citrate is about 200 mM.
50. The formulation of any of embodiments 1, 5, 7, 8, 9, 10, 11 or 13, wherein the concentration of polysorbate is between about 0.01% to about 0.05%.
51. The formulation of any of embodiments 1, 5, 7, 8, 9, 10, 11 or 13 wherein the polysorbate is selected from the group consisting of polysorbate-20, polysorbate-60, and polysorbate-80.
52. The formulation of any of embodiments 1 to 13, wherein the pH is between about 5.5 to about 7.0.
53. The formulation of embodiment 52, wherein the pH is between about 6.0 to about 7.0.
54. The formulation of any of embodiments 1 to 13, wherein the Fc variant protein comprises an Fc region with enhanced binding to an Fc receptor relative to a protein having the same amino acid sequence except having a naturally occurring Fc region.
55. The formulation of embodiment 54, wherein the Fc receptor is FcγRIIIA.
56. The formulation of embodiment 54, wherein the Fc receptor is FcRn
57. The formulation of any of embodiments 1 to 13, wherein the Fc variant protein comprises an Fc region with enhanced ADCC activity relative to a protein having the same amino acid sequence except having a naturally occurring Fc region.
58. The formulation of any of embodiments 1 to 13, wherein the Fc variant protein comprises an Fc region with enhanced serum half life relative to a protein having the same amino acid sequence except having a naturally occurring Fc region.
59. The formulation of any of embodiments 1 to 13, wherein the Fc variant protein comprises an Fc region having a non naturally occurring amino acid residue at one or more positions selected from the group consisting of: 222, 224, 234, 235, 236, 239, 240, 241, 243, 244, 245, 247, 248, 252, 254, 256, 258, 262, 263, 264, 265, 266, 267, 268, 269, 272, 274, 275, 278, 279, 280, 282, 290, 294, 295, 296, 297, 298, 299, 300, 312, 313, 318, 320, 325, 326, 327, 328, 329, 330, 332, 333, 334, 335, 339, 359, 360, 372, 377, 379, 396, 398, 400, 401, 430 and 436, as numbered by the EU index as set forth in Kabat.
60. The formulation of any of embodiments 1 to 13, wherein the Fc variant protein comprises an Fc region having at least one non naturally occurring amino acid residue selected from the group consisting of: 222N, 224L, 234D, 234E, 234N, 234Q, 234T, 234H, 234Y, 234I, 234V, 234F, 235A, 235D, 235R, 235W, 235P, 235S, 235N, 235Q, 235T, 235H, 235Y, 235I, 235V, 235F, 236E, 239D, 239E, 239N, 239Q, 239F, 239T, 239H, 239Y, 240I, 240A, 240T, 240M, 241W, 241L, 241Y, 241E, 241R, 243W, 243L, 243Y, 243R, 243Q, 244H, 245A, 247V, 247G, 248M, 252Y, 254T, 256E, 258D, 262I, 262A, 262T, 262E, 263I, 263A, 263T, 263M, 264L, 264I, 264W, 264T, 264R, 264F, 264M, 264Y, 264E, 265G, 265N, 265Q, 265Y, 265F, 265V, 265I, 265L, 265H, 265T, 266I, 266A, 266T, 266M, 267Q, 267L, 268D, 268N, 269H, 269Y, 269F, 269R, 296E, 272Y, 274E, 274R, 274T, 275Y, 278T, 279L, 280H, 280Q, 280Y, 282M, 290G, 290S, 290T, 290Y, 294N, 295K, 296Q, 296D, 296N, 296S, 296T, 296L, 296I, 296H, 269G, 297S, 297D, 297E, 298H, 298I, 298T, 298F, 299I, 299L, 299A, 299S, 299V, 299H, 299F, 299E, 300I, 300L, 312A, 313F, 318A, 318V, 320A, 320M, 325Q, 325L, 325I, 325D, 325E, 325A, 325T, 325V, 325H, 327G, 327W, 327N, 327L, 328S, 328M, 328D, 328E, 328N, 328Q, 328F, 328I, 328V, 328T, 328H, 328A, 329F, 329H, 329Q, 330K, 330G, 330T, 330C, 330L, 330Y, 330V, 330I, 330F, 330R, 330H, 332D, 332S, 332W, 332F, 332E, 332N, 332Q, 332T, 332H, 332Y, 332A, 335A, 335T, 335N, 335R, 335Y, 339T, 359A, 360A, 372Y, 377F, 379M, 396H, 396L, 398V, 400P, 401V, 430A, as numbered by the EU index as set forth in Kabat.
61. The formulation of embodiment 59, wherein the Fc region comprises a non naturally occurring amino acid at one or more positions selected from the group consisting of 239, 330 and 332, as numbered by the EU index as set forth in Kabat.
62. The formulation of embodiment 60, wherein the at least one non naturally occurring amino acid residue is selected from the group consisting of 239D, 330L, 330Y and 332E, as numbered by the EU index as set forth in Kabat.
63. The formulation of embodiment 60, wherein the Fc region comprises the non naturally occurring amino acids 239D, 330L and 332E, as numbered by the EU index as set forth in Kabat.
64. The formulation of embodiment 60, wherein the Fc region comprises the non naturally occurring amino acids 239D, 330Y and 332E, as numbered by the EU index as set forth in Kabat.
65. The formulation of embodiment 60, wherein the Fc region comprises the non naturally occurring amino acid 332E, as numbered by the EU index as set forth in Kabat.
66. The formulation of embodiment 59, wherein the Fc region further comprises a non naturally occurring amino acid at one or more positions selected from the group consisting of 252, 254, and 256, as numbered by the EU index as set forth in Kabat.
67. The formulation of embodiment 60, wherein the non naturally occurring amino acid at one or more positions are selected from the group consisting of 252Y, 254T and 256E, as numbered by the EU index as set forth in Kabat.
68. A method of reducing aggregation of an Fc variant protein comprising formulating said Fc variant protein in the formulation of any one of embodiments 1 to 14, 17 to 20, or 25 to 53.
69. The method of embodiment 68, wherein the aggretion of an Fc variant protein is reduced by at least 10% compared to the aggregation when the same Fc variant is formulated in 10 mM Histidine pH 6.0.
70. A pre-lyophilization bulk formulation comprising an Fc variant protein at a concentration between about 20 mg/mL and about 100 mg/mL, about 6% trehalose, about 2% arginine (115 mM), about 0.025% polysorbate-80 and about 10 mM histidine buffer, wherein said formulation has a pH of about 6.0.
71. The pre-lyophilization bulk formulation of claim 70, wherein the Fc variant protein comprises at least one of the amino acids sequences selected from the group consisting of SEQ ID NOS: 2, 4, 6 and 8-20.
72. A reconstituted formulation comprising an Fc variant protein at a concentration between about 40 mg/mL to about 100 mg/mL, between about 2% to about 12% trehalose, between about 1% to about 4% arginine or approximately 58 mM to 230 mM, and between about 5 mM to about 20 mM histidine buffer, wherein said reconstituted formulation has a pH of about 6.0.
73. The reconstituted formulation of embodiment 72, comprising an Fc variant protein at a concentration between about 40 mg/mL and about 100 mg/mL, about 12% trehalose, about 4% arginine or approximately 230 mM, about 20 mM histidine buffer, wherein said reconstituted formulation has a pH of 6.
74. The reconstituted formulation of embodiment 72, comprising an Fc variant protein at a concentration of about 40 mg/mL, about 4.5% trehalose, about 1.5% arginine or approximately 58 mM, about 7.5 mM histidine buffer, wherein said reconstituted formulation has a pH of 6.
75. The reconstituted formulation of embodiment 72, 73 or 74, wherein said Fc variant protein wherein the Fc variant protein comprises at least one of the amino acids sequences selected from the group consisting of SEQ ID NOS: 2, 4, 6 and 8-20.
76. The reconstituted formulation of embodiment 72, 73 or 74, wherein said reconstituted formulation further comprises about 0.01% to about 0.05% polysorbate 80.
77. A liquid formulation comprising an Fc variant protein at a concentration between about 20 mg/mL and about 100 mg/mL, about 50 mM to about 300 mM citrate, and about 10% to about 20% trehalose wherein, said formulation has a pH of between about 6.0 and about 7.0.
78. The liquid formulation of embodiment 77, wherein the concentration of citrate is about 50 mM and the concentration of trehalose is about 10%, wherein, said formulation has a pH of between about 6.0 and about 6.5.
79. The liquid formulation of embodiment 77, wherein the concentration of citrate is about 200 mM and the concentration of trehalose is about 10%.
80. The liquid formulation of embodiment 77, wherein the concentration of citrate is about 100 mM and the concentration of trehalose is about 15%.
81. The liquid formulation of embodiment 77, 79 or 80, wherein the pH is about 6.0.
82. The liquid formulation of embodiment 77, 79 or 80, wherein the pH is about 6.5.
83. The liquid formulation of embodiment 77, 79 or 80, wherein the pH is about 7.0.
84. The liquid formulation of embodiment 77, 79 or 80, further comprising a polysorbate at a concentration between about 0.001% to about 0.1%
85. A liquid formulation comprising an Fc variant protein at a concentration between about 20 mg/mL and 100 mg/mL, about 25 mM citrate, about 200 mM arginine, about 8% trehalose wherein, said formulation has a pH of between about 6.0 and about 6.5.
86. The liquid formulation of embodiment 85, further comprising a polysorbate at a concentration between about 0.001% to about 0.1%.
87. The liquid formulation of any of embodiments 77 to 85, wherein the Fc variant protein has at least 10% less aggregation when compared to the aggregation when the same Fc variant protein is formulated in 10 mM Histidine pH 6.0.
88. The liquid formulation of any of the embodiments 77 to 85, wherein the Fc variant protein has no more than about 2% aggregate, relative to total Fc variant protein at the temperature range of 2° C. to 8° C. for at least 90 days, as assessed by sized exclusion chromatograph (SEC).
89. The liquid formulation of any of embodiments 77 to 87, wherein the Fc variant protein is an antibody.
90. The liquid formulation of any of embodiments 77 to 87, wherein the Fc variant protein is an Fc fusion protein.
91. The liquid formulation of embodiment 89 or 90, wherein the Fc variant protein binds human EphA2 or human αVβ3 integrin.
92. The liquid formulation of embodiment 91, wherein the Fc variant protein comprises at least one of the amino acids sequences selected from the group consisting of SEQ ID NOS: 2, 4, 6 and 8-20.
93. The liquid formulation of embodiment 89 or 90, wherein the Fc variant protein binds the same antigen as a clinical product or candidate antibody selected from the group consisting of: rituximab, zanolimumab, hA20, AME-133, HumaLYM™, trastuzumab, pertuzumab, cetuximab, IMC-3G3, panitumumab, zalutumumab, nimotuzumab, matuzumab, ch806, KSB-102, MR1-1, SC100, SC101, SC103, alemtuzumab, muromonab-CD3, OKT4A, ibritumomab, gemtuzumab, alefacept, abciximab, basiliximab, palivizumab, motavizumab, infliximab, adalimumab, CDP-571, etanercept, ABX-CBL, ABX-IL8, ABX-MA1 pemtumomab, Therex, AS1405, natalizumab, HuBC-1, natalizumab, IDEC-131, VLA-1; CAT-152; J695, CAT-192, CAT-213, BR3-Fc, LymphoStat-B™, TRAIL-R1mAb, bevacizumab, ranibizumab, omalizumab, efalizumab, MLN-02, zanolimumab, HuMax-IL 15, HuMax-Inflam, HuMax-Cancer, HuMax-Lymphoma, HuMax-TAC, clenoliximab, lumiliximab, BEC2, IMC-1C11, DC101, labetuzumab, arcitumomab, epratuzumab, tacatuzumab, MyelomaCide™, LkoCide™, ProstaCide™, ipilimumab, MDX-060, MDX-070, MDX-018, MDX-1106, MDX-1103, MDX-1333, MDX-214, MDX-1100, MDX-CD4, MDX-1388, MDX-066, MDX-1307, HGS-TR2J, FG-3019, BMS-66513, SGN-30, SGN-40, tocilizumab, CS-1008, IDM-1, golimumab, CNTO 1275, CNTO 95, CNTO 328, mepolizumab, MOR101, MOR102, MOR201, visilizumab, HuZAF™, volocixmab, ING-1, MLN2201, daclizumab, HCD122, CDP860, PRO542, C14, oregovomab, edrecolomab, etaracizumab, siplizumab, lintuzumab, Hu1D10, Lym-1, efalizumab, ICM3, galiximab, eculizumab, pexelizumab, LDP-01, huA33, WX-G250, sibrotuzumab, Chimeric KW-2871, hu3S193, huLK26; bivatuzumab, ch14.18, 3F8, BC8, huHMFG1, MORAb-003, MORAb-004, MORAb-009, denosumab, PRO-140, 1D09C3, huMikbeta-1, NI-0401, NI-501, cantuzumab, HuN901, 8H9, chTNT-1/B, bavituximab, huJ591, HeFi-1, Pentacea™, abagovomab, tositumomab, 105AD7, GMA161 and GMA321.
94. The liquid formulation of embodiment 89 or 90, wherein the Fc variant protein competes for binding to the same antigen as a clinical product or candidate selected from the group consisting of: rituximab, zanolimumab, hA20, AME-133, HumaLYM™, trastuzumab, pertuzumab, cetuximab, IMC-3G3, panitumumab, zalutumumab, nimotuzumab, matuzumab, ch806, KSB-102, MR1-1, SC100, SC101, SC103, alemtuzumab, muromonab-CD3, OKT4A, ibritumomab, gemtuzumab, alefacept, abciximab, basiliximab, palivizumab, motavizumab, infliximab, adalimumab, CDP-571, etanercept, ABX-CBL, ABX-IL8, ABX-MA1 pemtumomab, Therex, AS1405, natalizumab, HuBC-1, natalizumab, IDEC-131, VLA-1; CAT-152; J695, CAT-192, CAT-213, BR3-Fc, LymphoStat-B™, TRAIL-R1mAb, bevacizumab, ranibizumab, omalizumab, efalizumab, MLN-02, zanolimumab, HuMax-IL 15, HuMax-Inflam, HuMax-Cancer, HuMax-Lymphoma, HuMax-TAC, clenoliximab, lumiliximab, BEC2, IMC-1C11, DC101, labetuzumab, arcitumomab, epratuzumab, tacatuzumab, MyelomaCide™, LkoCide™, ProstaCide™, ipilimumab, MDX-060, MDX-070, MDX-018, MDX-1106, MDX-1103, MDX-1333, MDX-214, MDX-1100, MDX-CD4, MDX-1388, MDX-066, MDX-1307, HGS-TR2J, FG-3019, BMS-66513, SGN-30, SGN-40, tocilizumab, CS-1008, IDM-1, golimumab, CNTO 1275, CNTO 95, CNTO 328, mepolizumab, MOR101, MOR102, MOR201, visilizumab, HuZAF™, volocixmab, ING-1, MLN2201, daclizumab, HCD122, CDP860, PRO542, C14, oregovomab, edrecolomab, etaracizumab, siplizumab, lintuzumab, Hu1D10, Lym-1, efalizumab, ICM3, galiximab, eculizumab, pexelizumab, LDP-01, huA33, WX-G250, sibrotuzumab, Chimeric KW-2871, hu3S193, huLK26; bivatuzumab, ch14.18, 3F8, BC8, huHMFG1, MORAb-003, MORAb-004, MORAb-009, denosumab, PRO-140, 1D09C3, huMikbeta-1, NI-0401, NI-501, cantuzumab, HuN901, 8H9, chTNT-1/B, bavituximab, huJ591, HeFi-1, Pentacea™, abagovomab, GMA161 and GMA321.
95. The liquid formulation of embodiment 89 or 90, wherein the Fc variant protein comprises at least one CDR from an antibody selected from the group consisting of: rituximab, zanolimumab, hA20, AME-133, HumaLYM™, trastuzumab, pertuzumab, cetuximab, IMC-3G3, panitumumab, zalutumumab, nimotuzumab, matuzumab, ch806, KSB-102, MR1-1, SC100, SC101, SC103, alemtuzumab, muromonab-CD3, OKT4A, ibritumomab, gemtuzumab, alefacept, abciximab, basiliximab, palivizumab, motavizumab, infliximab, adalimumab, CDP-571, ABX-CBL, ABX-IL8, ABX-MA1 pemtumomab, Therex, AS1405, natalizumab, HuBC-1, natalizumab, IDEC-131, VLA-1; CAT-152; J695, CAT-192, CAT-213, LymphoStat-B™, TRAIL-R1mAb, bevacizumab, ranibizumab, omalizumab, efalizumab, MLN-02, zanolimumab, HuMax-IL 15, HuMax-Inflam, HuMax-Cancer, HuMax-Lymphoma, HuMax-TAC, clenoliximab, lumiliximab, BEC2, IMC-1C11, DC101, labetuzumab, arcitumomab, epratuzumab, tacatuzumab, MyelomaCide™, LkoCide™, ProstaCide™, ipilimumab, MDX-060, MDX-070, MDX-018, MDX-1106, MDX-1103, MDX-1333, MDX-214, MDX-1100, MDX-CD4, MDX-1388, MDX-066, MDX-1307, HGS-TR2J, FG-3019, BMS-66513, SGN-30, SGN-40, tocilizumab, CS-1008, IDM-1, golimumab, CNTO 1275, CNTO 95, CNTO 328, mepolizumab, MOR101, MOR102, MOR201, visilizumab, HuZAF™, volocixmab, ING-1, MLN2201, daclizumab, HCD122, CDP860, PRO542, C14, oregovomab, edrecolomab, etaracizumab, siplizumab, lintuzumab, Hu1D10, Lym-1, efalizumab, ICM3, galiximab, eculizumab, pexelizumab, LDP-01, huA33, WX-G250, sibrotuzumab, Chimeric KW-2871, hu3S193, huLK26; bivatuzumab, ch14.18, 3F8, BC8, huHMFG1, MORAb-003, MORAb-004, MORAb-009, denosumab, PRO-140, 1D09C3, huMikbeta-1, NI-0401, NI-501, cantuzumab, HuN901, 8H9, chTNT-1/B, bavituximab, huJ591, HeFi-1, Pentacea™, abagovomab, tositumomab, 105AD7, GMA161 and GMA321.
96. The liquid formulation of any of embodiments 77 to 86, wherein the Fc variant protein comprises an Fc region with enhanced binding to an Fc receptor relative to a protein having the same amino acid sequence except having a naturally occurring Fc region.
97. The liquid formulation of embodiment 96, wherein the Fc receptor is FcγRIIIA.
98. The liquid formulation of embodiment 96, wherein the Fc receptor is FcRn.
99. The liquid formulation of any of embodiments 77 to 86, wherein the Fc variant protein comprises an Fc region with enhanced ADCC activity relative to a protein having the same amino acid sequence except having a naturally occurring Fc region.
100. The liquid formulation of any of embodiments 77 to 86, wherein the Fc variant protein comprises an Fc region with enhanced serum half life relative to a protein having the same amino acid sequence except having a naturally occurring Fc region.
101. The liquid formulation of any of embodiments 77 to 86, wherein the Fc variant protein comprises an Fc region having a non naturally occurring amino acid residue at one or more positions selected from the group consisting of: 222, 224, 234, 235, 236, 239, 240, 241, 243, 244, 245, 247, 248 252, 254, 256, 258, 262, 263, 264, 265, 266, 267, 268, 269, 272, 274, 275, 278, 279, 280, 282, 290, 294, 295, 296, 297, 298, 299, 300, 312, 313, 318, 320, 325, 326, 327, 328, 329, 330, 332, 333, 334, 335, 339, 359, 360, 372, 377, 379, 396, 398, 400, 401, 430, and 436 as numbered by the EU index as set forth in Kabat.
102. The liquid formulation of any of embodiments 77 to 86, wherein the Fc variant protein comprises an Fc region having at least one non naturally occurring amino acid residue selected from the group consisting of: 222N, 224L, 234D, 234E, 234N, 234Q, 234T, 234H, 234Y, 234I, 234V, 234F, 235A, 235D, 235R, 235W, 235P, 235S, 235N, 235Q, 235T, 235H, 235Y, 235I, 235V, 235F, 236E, 239D, 239E, 239N, 239Q, 239F, 239T, 239H, 239Y, 2401, 240A, 240T, 240M, 241W, 241L, 241Y, 241E, 241R, 243W, 243L, 243Y, 243R, 243Q, 244H, 245A, 247V, 247G, 248M, 252Y, 254T, 256E, 258D, 262I, 262A, 262T, 262E, 263I, 263A, 263T, 263M, 264L, 264I, 264W, 264T, 264R, 264F, 264M, 264Y, 264E, 265G, 265N, 265Q, 265Y, 265F, 265V, 265I, 265L, 265H, 265T, 266I, 266A, 266T, 266M, 267Q, 267L, 268D, 268N, 269H, 269Y, 269F, 269R, 296E, 272Y, 274E, 274R, 274T, 275Y, 278T, 279L, 280H, 280Q, 280Y, 282M, 290G, 290S, 290T, 290Y, 294N, 295K, 296Q, 296D, 296N, 296S, 296T, 296L, 296I, 296H, 269G, 297S, 297D, 297E, 298H, 298I, 298T, 298F, 299I, 299L, 299A, 299S, 299V, 299H, 299F, 299E, 300I, 300L, 312A, 313F, 318A, 318V, 320A, 320M, 325Q, 325L, 325I, 325D, 325E, 325A, 325T, 325V, 325H, 327G, 327W, 327N, 327L, 328S, 328M, 328D, 328E, 328N, 328Q, 328F, 328I, 328V, 328T, 328H, 328A, 329F, 329H, 329Q, 330K, 330G, 330T, 330C, 330L, 330Y, 330V, 330I, 330F, 330R, 330H, 332D, 332S, 332W, 332F, 332E, 332N, 332Q, 332T, 332H, 332Y, 332A, 335A, 335T, 335N, 335R, 335Y, 339T, 359A, 360A, 372Y, 377F, 379M, 396H, 396L, 398V, 400P, 401V, 430A, as numbered by the EU index as set forth in Kabat.
103. The liquid formulation of embodiment 101, wherein the Fc region comprises a non naturally occurring amino acid at one or more positions selected from the group consisting of 239, 330 and 332, as numbered by the EU index as set forth in Kabat.
104. The liquid formulation of embodiment 102, wherein the at least one non naturally occurring amino acid residue is selected from the group consisting of 239D, 330L, 330Y and 332E, as numbered by the EU index as set forth in Kabat.
105. The liquid formulation of embodiment 102, wherein the Fc region comprises the non naturally occurring amino acids 239D, 330L and 332E, as numbered by the EU index as set forth in Kabat.
106. The liquid formulation of embodiment 102, wherein the Fc region comprises the non naturally occurring amino acids 239D, 330Y and 332E, as numbered by the EU index as set forth in Kabat.
107. The liquid formulation of embodiment 102, wherein the Fc region comprises the non naturally occurring amino acid 332E, as numbered by the EU index as set forth in Kabat.
108. The liquid formulation of embodiment 101, wherein the Fc region further comprises a non naturally occurring amino acid at one or more positions selected from the group consisting of 252, 254, and 256, as numbered by the EU index as set forth in Kabat.
109. The liquid formulation of embodiment 102, wherein the non naturally occurring amino acid at one or more positions are selected from the group consisting of 252Y, 254T and 256E, as numbered by the EU index as set forth in Kabat.

### 8. EXAMPLES

The invention is now described with reference to the following examples. These examples are provided for the purpose of illustration only and the invention should in no way be construed as being limited to these examples but rather should be construed to encompass any and all variations which become evident as a result of the teachings provided herein.

### 8.1 Example 1

### Stability Analysis of Fc Variants

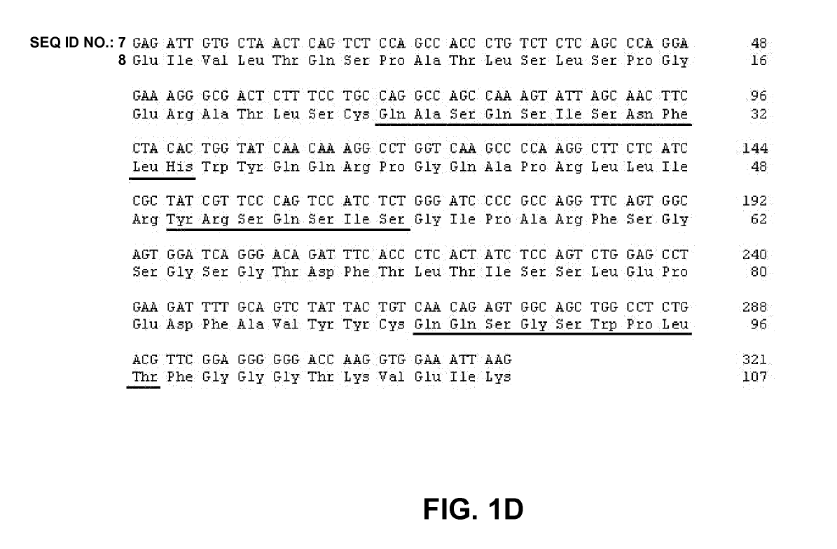

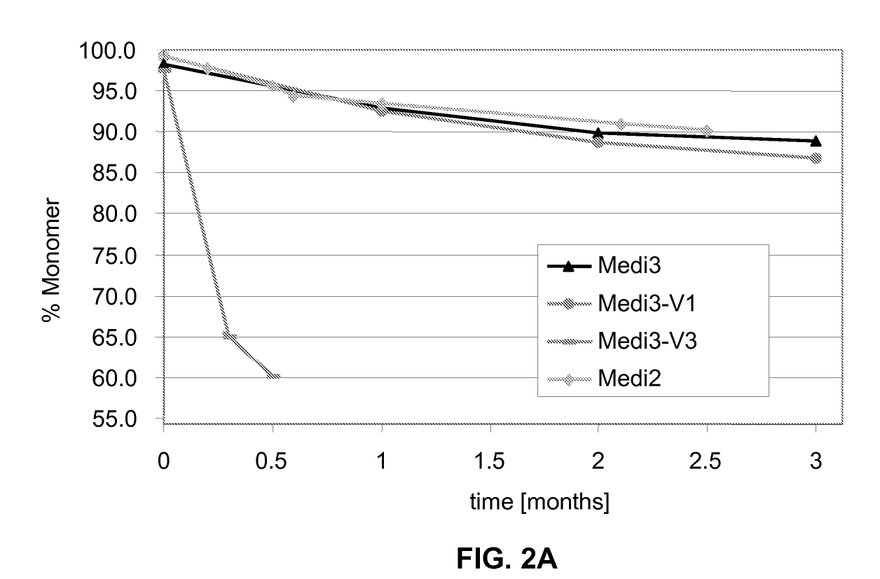

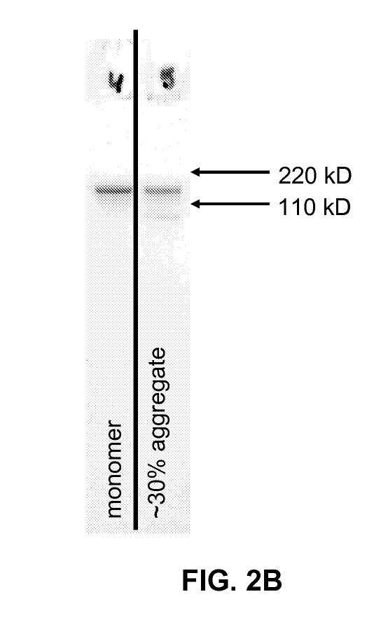

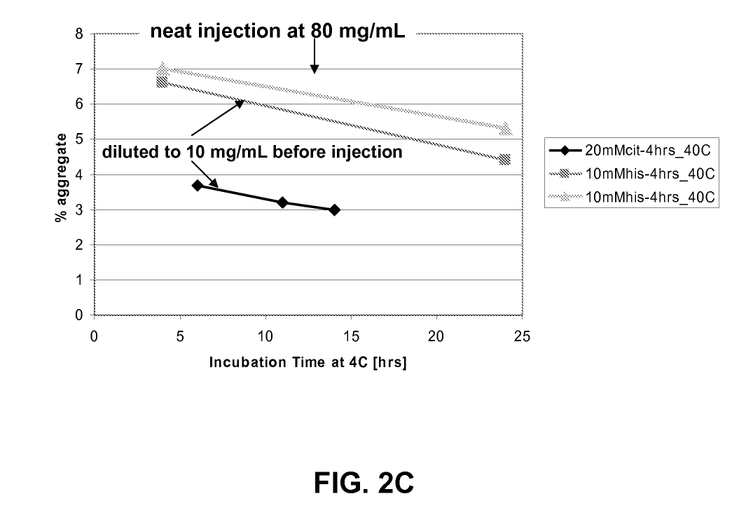

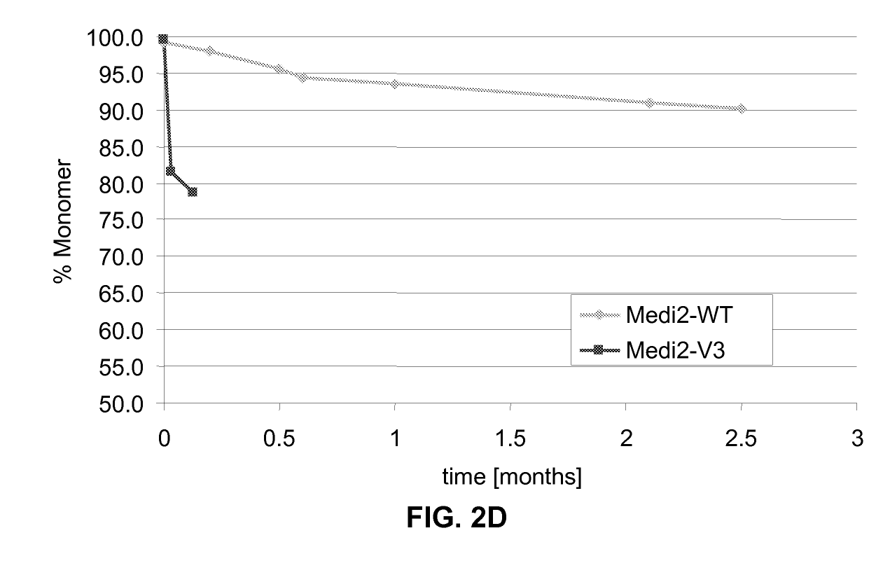

Two Fc variants of an anti-EphA2 antibody (designated “Medi3”, see FIG. 1A-B for variable region, see Table 2 for SEQ ID NOS.) were generated. Variant 1 (designated “Medi3-V1”) has a glutamate at residue 332 as numbered by the EU index as set forth in Kabat, and has a binding affinity for FcγRIIIA that is 8.8 fold higher than Medi3. Variant 2 (designated “Medi3-V3”) has an aspartate at amino at residue 239, a leucine at residue 330, and a glutamate at residue 332 as numbered by the EU index as set forth in Kabat, and has a binding affinity for FcγRIIIA that is nearly 100 fold higher than Medi3 (data not shown). Medi3-V1 and Medi3-V3 also have higher ADCC activity compared to wild type Medi3, the relative ADCC activity was Medi3-3V>Medi3-1V>Medi3 (data not shown). The wild type Medi3 antibody and the Fc variants as well as a second wild type anti-Integrin αVβ3 antibody (designated “Medi2”, see FIG. 1C-D for variable region see Table 2 for SEQ ID NOS.), having a distinct variable region, were formulated at 100 mg/mL in 10 mM histidine buffer, pH 6.0 and the samples were analyzed by size exclusion chromatography with UV detection over a three month period when stored at 40° C. Alternatively, the antibody formulations may be analyzed for the presence of antibody aggregates and/or fragments by capillary gel electrophoresis methods such as those described below. Additionally, materials generated may be analyzed using polyacrylamide gel electrophoresis methods such as those described below, to monitor for purity, nature of the aggregate-covalent or noncovalent, and also the presence/absence of any fragment/s. The percent of monomer present in the formulations plotted over time is shown in FIG. 2A. A reduction in the amount of monomer present correlates with aggregation of the antibody in the formulation and is indicative of reduced stability. The amount of monomer present in both wild type antibody formulations, as well as the Medi3-V1 formulation were comparable, showing a reduction of less than 15% over the three month period. In contrast, the percent of monomer in the Medi3-V3 formulation dropped by 40% in just 15 days. Indicating that the Medi3-V3 antibody is unstable as formulated compared to the WT version (Medi3) and a WT version of an unrelated antibody (Medi2).

A solution of Medi3-V3 having little or no aggregation was examined by SDS-PAGE under non-reducing conditions and found to run predominantly as a single monomer band (FIG. 2B, lane 4). Similarly, a solution having 30% aggregates (as determined by SEC, data not shown) was also seen to run predominantly as a monomer band under these conditions (FIG. 2B, lane 5), indicating that the aggregates are not covalent in nature and indicated that the aggregation may be reversible. SEC analysis was used to examine the reversibility of aggregates formed by incubation of concentrated antibody at 40° C. A 2% reduction in % aggregates formed at 40° C. in an 80 mg/ml solution was seen over a 16 hour incubation at 4° C. (see FIG. 2C, triangles). A similar reduction was seen when the solution was first diluted to 10 mg/ml and then incubated at 4° C. (FIG. 2C, squares) however, the % aggregation at the earliest time point examined (4 hrs) was already reduced compared to the undiluted antibody solution (compare triangles and squares). A larger initial decrease in % aggregates (˜3%) was seen after 6 hours at 4° C. when the aggregated antibody solution was diluted to 10 mg/ml in a buffer comprising 20 mM Citrate the % aggregates continued to decrease upon longer incubation at 4° C. (FIG. 2C, diamonds). Together these data indicate that the aggregation of Medi3-V3 is non-covalent and somewhat reversible under certain incubation conditions.

To further investigate the role of the variant Fc region in reducing the stability of Medi3-V3 a “V3” Fc variant of the Medi2 antibody, having an aspartate at amino at residue 239, a leucine at residue 330, and a glutamate at residue 332 as numbered by the EU index as set forth in Kabat, was also generated (designated “Medi2-V3”). Like Medi2-V3, Medi2-V3 has a much higher binding affinity for FcγRIIIA, 78 fold higher than Medi2, and higher ADCC activity (data not shown). The stability of Medi2-V3 formulated at 80 mg/mL in 10 mM histidine buffer, pH 6.0 and stored at 40° C. was analyzed by size exclusion chromatography with UV detection over a 24 hour period. The percent of monomer present in the formulations plotted over time is shown in FIG. 2D. The percent of monomer present in solution drops by 20.7% in just 4 days while the concentration of Medi2 (WT) monomer dropped by only about 9.0% after 2.5 months.

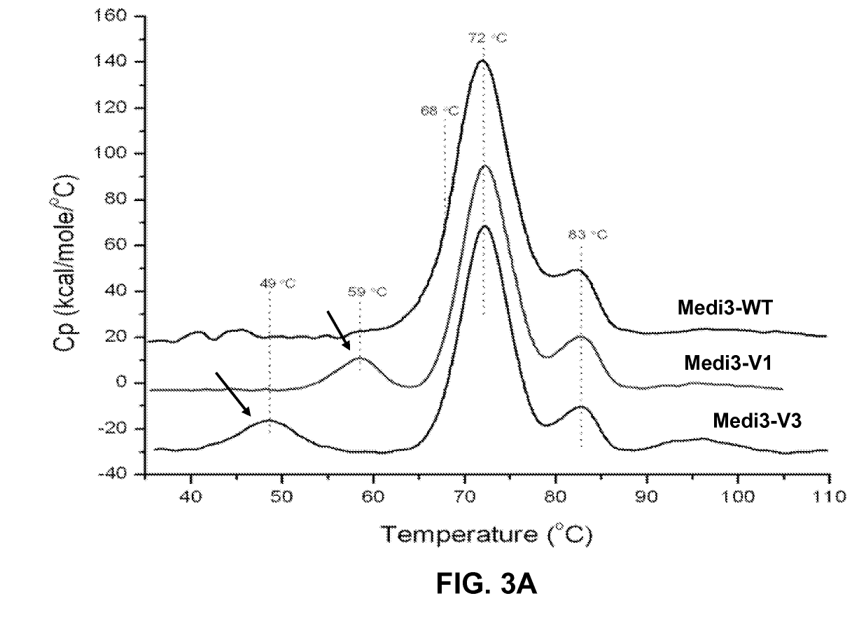

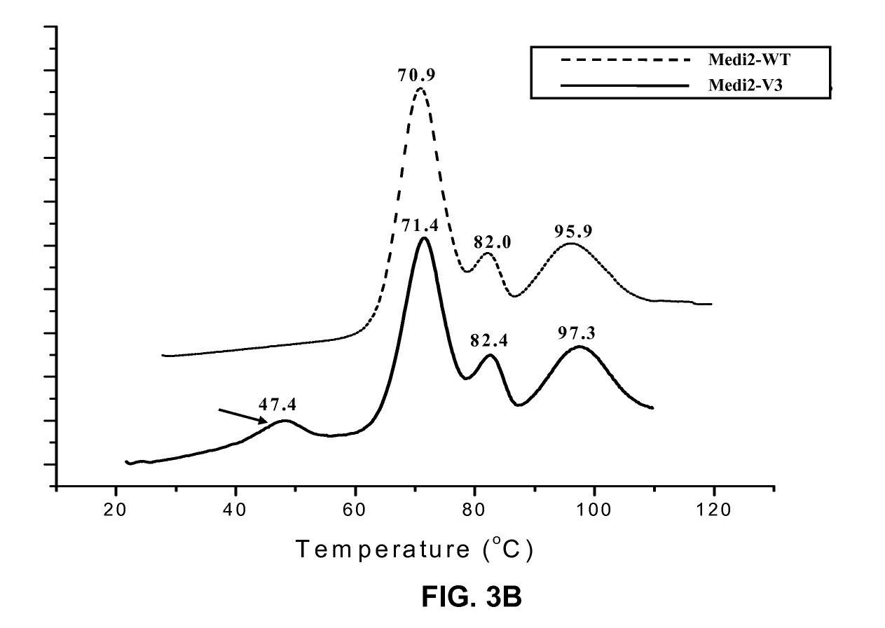

To characterize the role of the variant Fc region in reducing antibody stability Differential Scanning Calorimetry (DSC) was used to examine the melting curves of Medi3 and the two Fc variants (FIG. 3). Previous studies have demonstrated that the largest peak in these curves is generated by the melting of the variable domain, thus the variable domains of Medi3 and both variants all have a melting temperature (Tm) of ˜72° C. In studies not shown, the Tm of wild type Fc region, the C_{H}² domain specifically, was determined to be ˜69° C., a discrete peak for the Fc region of Medi3 can not be seen due to overlap with the curve generated by the melting of the variable region. However, the peak can be seen for both of the Fc variants. The Tm of the Fc region drops to ˜59° C. for Medi3-V1 and a further reduction to only ˜49° C. is seen for Medi3-V3. Together, these results demonstrate that the variant Fc region, the C_{H}² domain in particular, is largely responsible for the increased aggregation of the Medi3-V3 and Medi2-V3 Fc variants due in part to the decrease in Tm for the variant Fc region.

TABLE 2
Sequences

| Description | SEQ ID NO. |
|---|---|
| Anti-EphA2 antibody Medi3 heavy chain variable | 1 |
| region (nucleotide) | |
| Anti-EphA2 antibody Medi3 heavy chain variable | 2 |
| region (amino acid) | |
| Anti-EphA2 antibody Medi3 light chain variable | 3 |
| region (nucleotide) | |
| Anti-EphA2 antibody Medi3 light chain variable | 4 |
| region (amino acid) | |
| Anti-integrin αVβ3 antibody Medi2 heavy chain | 5 |
| variable region (nucleotide) | |
| Anti-integrin αVβ3 antibody Medi2 heavy chain | 6 |
| variable region (amino acid) | |
| Anti-integrin αVβ3 antibody Medi2 light chain | 7 |
| variable region (nucleotide) | |
| Anti-integrin αVβ3 antibody Medi2 light chain | 8 |
| variable region (amino acid) | |
| Anti-EphA2 antibody Medi3 heavy chain CDR1 | 9 |
| Anti-EphA2 antibody Medi3 heavy chain CDR2 | 10 |
| Anti-EphA2 antibody Medi3 heavy chain CDR3 | 11 |
| Anti-EphA2 antibody Medi3 light chain CDR1 | 12 |
| Anti-EphA2 antibody Medi3 light chain CDR2 | 13 |
| Anti-EphA2 antibody Medi3 light chain CDR3 | 14 |
| Anti-integrin αVβ3 antibody Medi2 heavy chain CDR1 | 15 |
| Anti-integrin αVβ3 antibody Medi2 heavy chain CDR2 | 16 |
| Anti-integrin αVβ3 antibody Medi2 heavy chain CDR3 | 17 |
| Anti-integrin αVβ3 antibody Medi2 light chain CDR1 | 18 |
| Anti-integrin αVβ3 antibody Medi2 light chain CDR2 | 19 |
| Anti-integrin αVβ3 antibody Medi2 light chain CDR3 | 20 |

### 8.1.1 Methods

Size Exclusion Chromatography (SEC): Size exclusion chromatography was performed to analyze the antibody formulation for the presence of antibody aggregates and fragments. The test samples were injected onto a size exclusion G3000 SW_{XL} column (5 μm, 300 Å, 7.8×300 mm, TosoHaas). The mobile phase was 0.1 M di-sodium phosphate, 0.1 M sodium sulfate and 0.05% sodium azide (pH 6.8), running isocratically at a flow rate of 1.0 mL/min. Eluted protein was detected by UV absorbance at 280 nm and collected for further characterization. The relative amount of any protein species detected was reported as the area percent of the product peak as compared to the total area of all other detected peaks excluding the initial included volume peak. Peaks eluting earlier than the antibody monomer peak were recorded in the aggregate percentile, while peaks eluting later than the antibody monomer peak, but earlier than the buffer peak, were recorded in the fragment percentile.

Capillary Gel Electrophoresis Using Sodium Dodecyl Sulfate (CGE-SDS): CGE-SDS are performed in an extended light path capillary (Agilent Technologies) of 50 μm i.d. and with a total length of 38.5 cm. Analyses are performed using a Hewlett Packard 3D-capillary electrophoresis unit. UV detection is conducted at 220 nm. Reagents-SDS sample buffer, SDS running buffer, and 2-mercaptoethanol may be purchased from a commercial source. Sample Preparation. Samples are diluted to 2.5 mg/mL in water. For reduced samples, 80 μL of diluted antibody are mixed with 100 μL of CE-SDS sample buffer and 20 μL of neat 2-mercaptoethanol. For nonreduced samples, the 20 μL of 2-mercaptoethanol is replaced with water. Reduced samples are incubated in a boiling water bath for 10 minutes. Nonreduced samples are not heated. CE Analysis. Prior to injection, the capillary is rinsed with 0.1 M NaOH, 0.1 M HCl, and SDS running buffer for 3, 3, and 8 minutes respectively. Samples are injected electrophoretically for 40 seconds at −10 kV. The CE analysis is conducted in the negative polarity mode (−15 kV). Typical current obtained is 20 μA. Capillary temperature is maintained at 50° C. and samples are at ambient temperatures.

Polyacrylamide Gel Electrophoresis: NuPAGE gels (Invitrogen) are used containing 4-12% Bis-Tris. Analysis involves running samples under both reduced (heating at 70° C. for 10 mins) and non-reduced conditions (no heating). For the NR Sample—5 ul of the sample at 5 mg/ml, 60 ul of reverse osmosis deionized (RODI) water, 25 ul of Sample Buffer (total Volume=90 ul). Then 10 ul of the RODI water is added to bring the sample to a total volume of 100 ul. For the R Sample-5 ul of the sample at 5 mg/ml, 60 ul of RODI water, 25 ul of Sample Buffer (total Volume=90 ul). Then 10 ul of reducing agent is added to the sample to a total volume of 100 ul. These samples are then heated to 70° C. for 10 mins. 15 ul of sample is loaded per well. Gels are run in 1×MES running buffer at 200 V for 35 minutes. 500 ul of antioxidant is added to the inner chamber of the reduced gel. An electrophoresis marker (e.g., color burst, Sigma) is used to cover from a broad range i.e. 220 kDa to 8 kDa. The gels are stained, for example with Simply Blue Safe Stain and preserved for example with Gel-Dry solution.

Differential Scanning Calorimetrv (DSC): Thermal melting temperatures (Tₘ) were measured with a VP-DSC (MicroCal, LLC) using a scan rate of 1.0° C./min and a temperature range of 10-110° C. or 25-120° C. A filter period of 8 seconds was used along with a 5 minute pre-scan thermostating. Samples were prepared by dialysis into 10 mM Histidine-HCl, pH 6 or into Formulation, by dialysis (e.g., using Pierce dialysis cups (3.5 kD)). Average mAb concentrations were 50 μg/mL as determined by A₂₈₀. Melting temperatures were determined following manufacturer procedures using Origin software supplied with the system. Briefly, multiple baselines were run with buffer in both the sample and reference cell to establish thermal equilibrium. After the baseline was subtracted from the sample thermogram, the data were concentration normalized and fitted using the deconvolution function. Tₘs are reported at the endothermic peak maximum of heat capacity in the thermograms obtained.

### 8.2 Example 2

### Effect of Concentration and Temperature of Fc Variant Stability

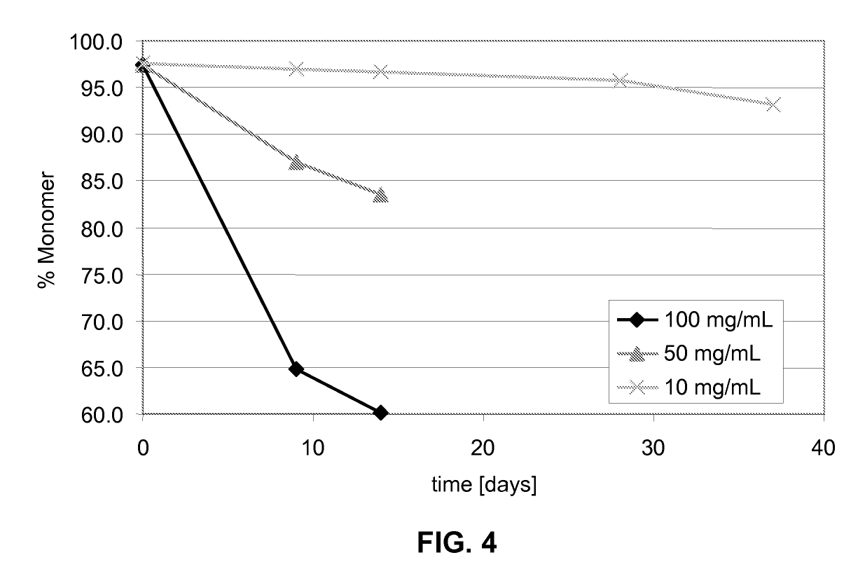

The stability of Medi3-V3 formulated in 10 mM histidine buffer, pH 6.0 at several different concentrations (10, 50 and 100 mg/mL) when stored at 40° C. was analyzed by size exclusion chromatography (SEC) with UV detection (as described above) over a 37 day period and the percent of monomer present in the formulations is plotted over time (FIG. 4). The percent monomer present in the 10 mg/mL solution decreased by only about 4.4% at day 37 while the 50 mg/mL and 100 mg/mL solutions showed about a 14% and 37.5% decrease, respectively after just 14 days indicating that aggregation is increased in higher concentration solutions.

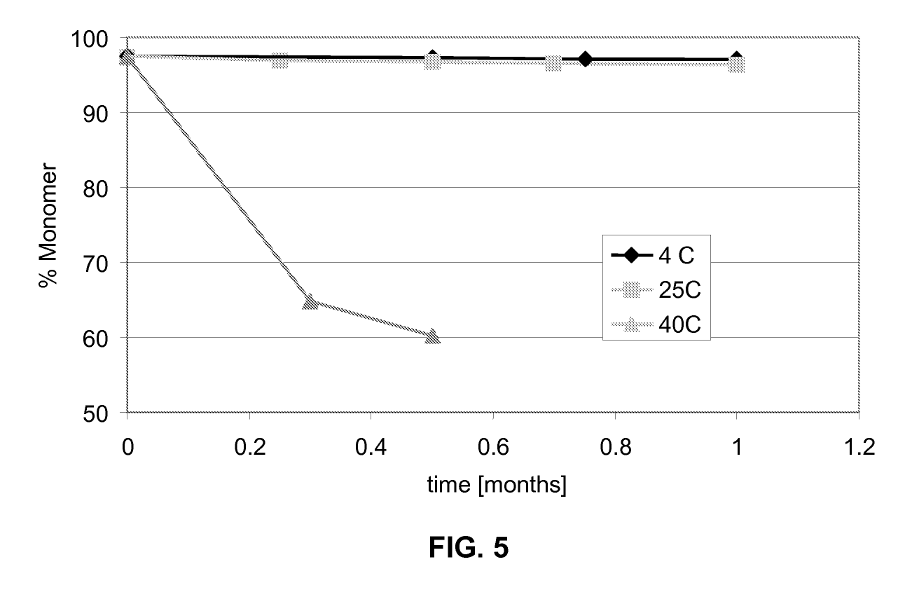

The stability of Medi3-V3 formulated in 10 mM histidine buffer, pH 6.0 at 100 mg/mL and stored at several different temperatures (4, 25 and 40° C.) was analyzed by size exclusion chromatography with UV detection over a 30 day period and the percent of monomer present in the formulations is plotted over time (FIG. 5). The percent monomer present in the solutions stored at 4° C. and 25° C. decreased by 0.3% and 1.0%, respectively after 30 days while the percent monomer decreased by about 37% in the solution stored at 40° C. after just 15 days.

These data indicate that while the “V3” variants are more stable at lower concentrations and/or lower temperatures, the overall stability of the V3 antibodies in 10 mM histidine buffer, pH 6.0 is not optimal for production and fill finish of a high concentration antibody formulation.

### 8.3 Example 3

### Fast Screen Assay of Buffer Formulations

A “Fast Screen” assay method was developed to rapidly screen a large number of different buffer formulations for those which improved the stability of V3 variants. Briefly, 100 mg/mL solution of Medi3-V3 in 10 mM histidine buffer, pH 6.0 was used as a monoclonal antibody (mAb) stock solution. The method utilizes concentrated excipient solutions which are added at 20% volume into an aliquot of the mAb stock solution. After excipient spiking, the protein concentration was 80 mg/mL. These excipient containing mAb solutions were incubated at 40° C. for 4-24 hours, and aggregate content was measured by SEC (as described above). The “Percent (%) Loss in Purity” (virtually the same as increase in percent aggregate) was used as an indicator to compare the stabilization imparted by excipients. A series of excipients were screened using this assay as described below.

### 8.3.1 Sugars and Arginine

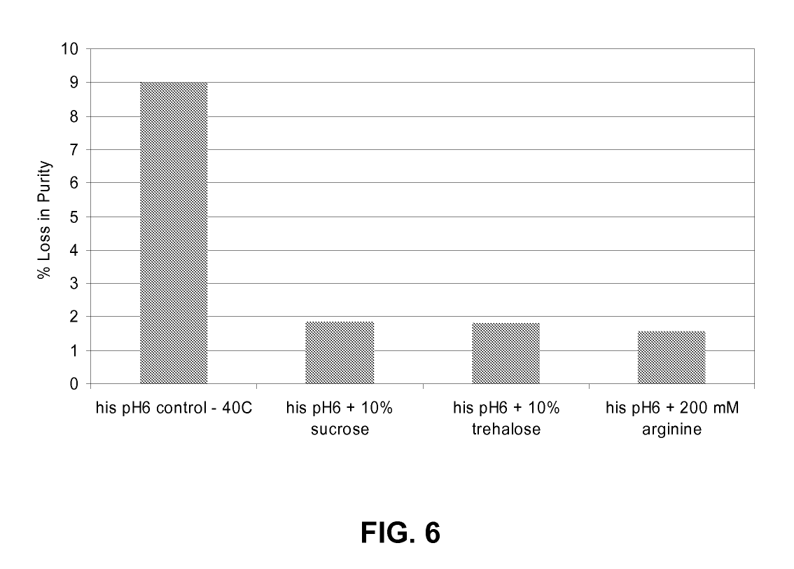

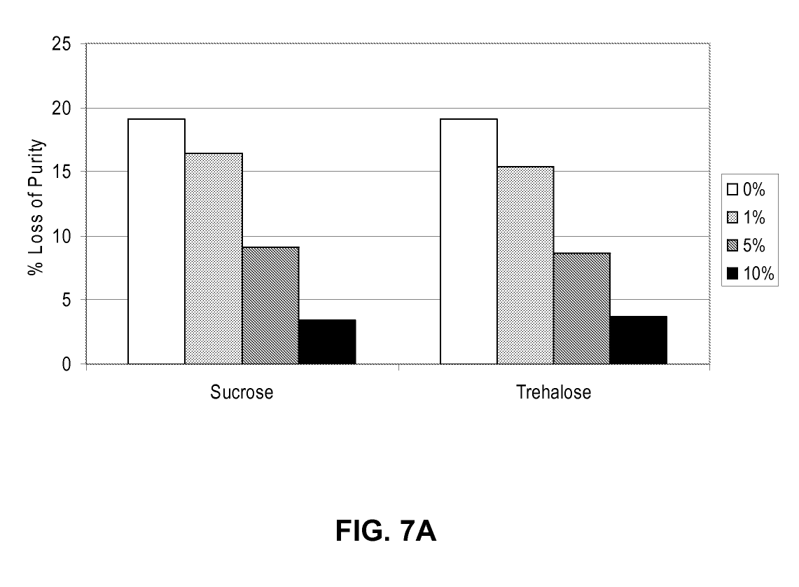

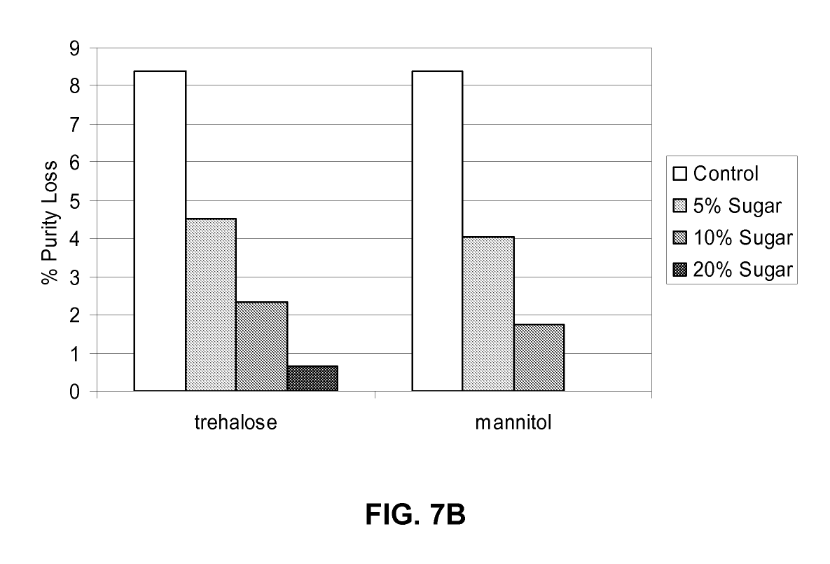

10% sucrose, 10% trehalose, and 200 mM L-Arginine each provided significant stabilization reducing the percent loss in purity over a 7 hour incubation from about 9% in the control (10 mM histidine buffer, pH 6.0) to less than 2% (FIG. 6). The effect of sucrose and trehalose was compared at a variety of concentrations. The percent loss in purity over a 24 hour incubation period was about 19% in the control and about 16%, 9% and 3% in the samples containing 1%, 5% and 10% sugar, respectively (FIG. 7A). Increasing concentration of either sugar lead to increased stabilization and both sugars had a nearly identical stabilization effect indicating that they are likely interchangeable.

Similarly, the stabilizing effect of trehalose and mannitol on a 50 mg/ml antibody solution (20 mM histidine buffer, pH 6) was compared at a variety of concentrations (5%-20%). The samples were incubated for 1 day at 40° C. and the percent loss in purity was determined by SEC (as described above). The percent loss in purity over a 24 hour incubation period was about 8.4% in the control and about 4%, 2% and 0.6% in the samples containing 5%, 10% and 20% sugar, respectively (FIG. 7B). As was seen before, increasing concentration of either sugar lead to increased stabilization and at 5-10% all three sugars had a nearly identical stabilization effect indicating that these three sugars are likely interchangeable. Only trehalose was tested at concentrations above 20% however, similar improvements in stability would be expected with any of the sugars tested.

### 8.3.2 Amino Acids, Anionic Species and Chelating Agent

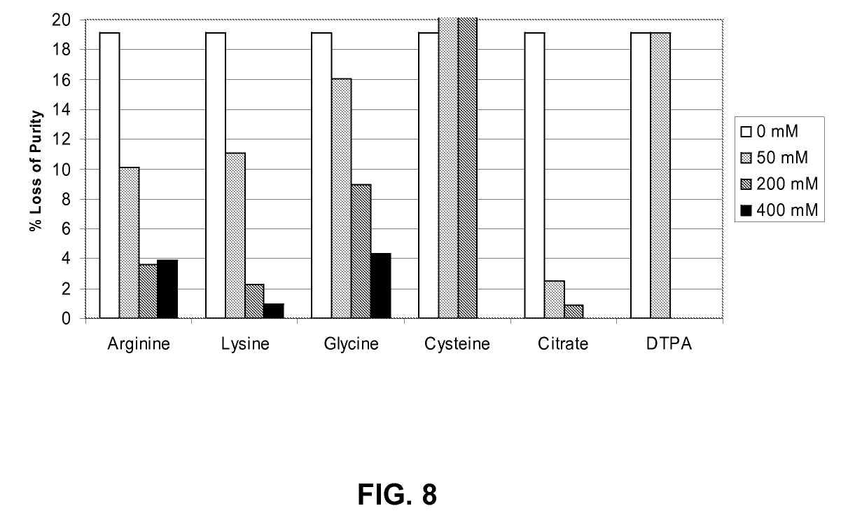

L-Arginine and several additional amino acids were tested at 50 mM, 200 mM and 400 mM concentrations along with the anionic species, citrate. In addition, the chelating agent DTPA was tested at 50 mM. As shown in FIG. 8A, arginine, lysine, and citrate each provided significant stabilization reducing the percent loss in purity over a 24 hour incubation from 19% in the control (10 mM histidine buffer, pH 6.0) to 4% or less at concentrations of 200 mM to 400 mM. Glycine was somewhat less effective, reducing the percent loss in purity to about 9% or less at concentrations of 200 mM to 400 mM. The relative ranking was seen to be citrate>lysine>arginine>glycine, where “>” is used to mean “has greater stabilizing impact than”. DTPA was not seen to have any effect while cysteine caused a large increase in aggregates (see Example 7 below).

### 8.3.3 Combined Effect of Sucrose and L-Arginine

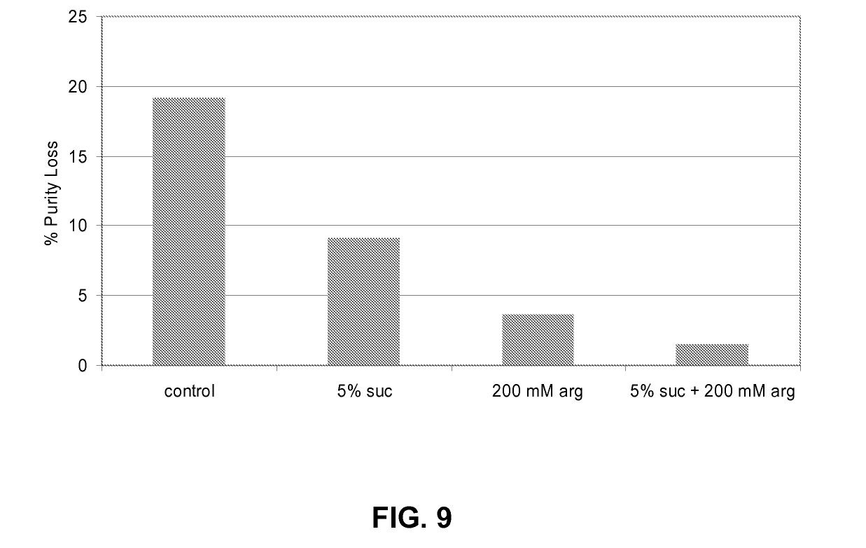

5% Sucrose alone reduced the percent loss in purity over a 24 hour incubation from about 19% in the control (10 mM histidine buffer, pH 6.0) to about 9% while 200 mM L-arginine reduced the percent loss in purity to about 3.5% (FIG. 9). However, the combination of 5% sucrose and 200 mM L-arginine was even more effective than each component independently, reducing the percent loss in purity to just 1.5% (FIG. 9).

### 8.3.4 Additional Members of Several Classes of Molecules

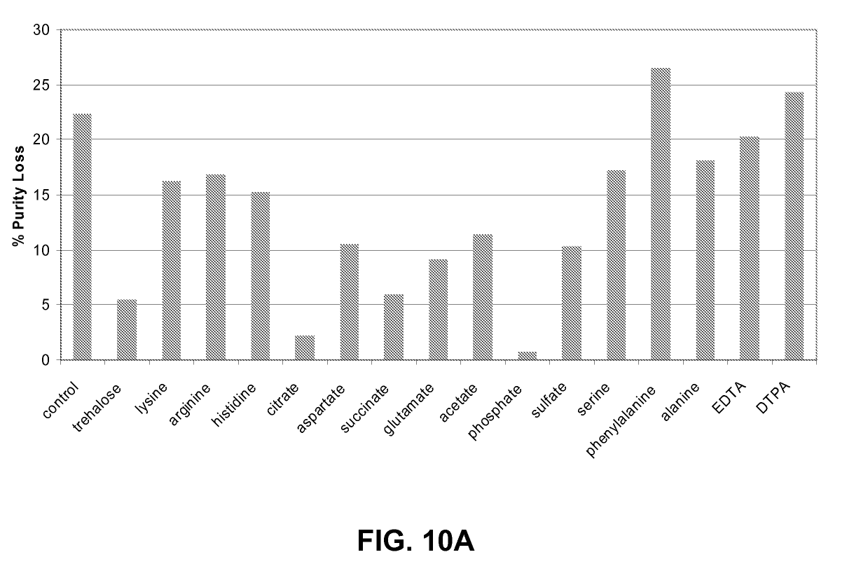

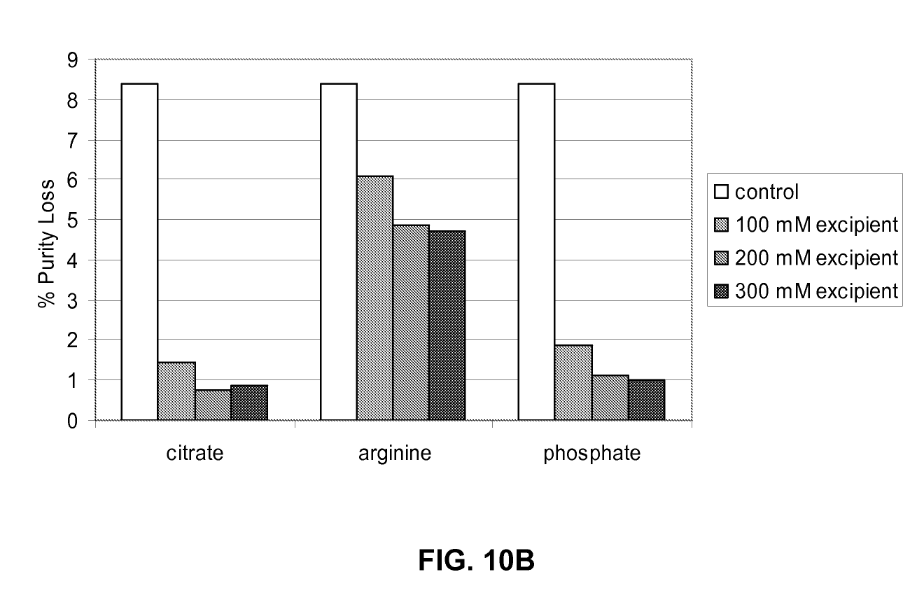

To expand on the excipients tested above, several additional members of each class of molecule were examined. In addition, several additional classes of molecules were tested. The samples were incubated for 19 hours at 40° C. and the percent loss in purity was determined by SEC (as described above). Trehalose was tested again at 10% and found to reduce the percent loss in purity over a 19 hour incubation from about 22% in the control (10 mM histidine buffer, pH 6.0) to about 5.4% (FIG. 10).

The anionic molecules citrate, aspartate, succinate, glutamate, acetate, phosphate, and sulfate were found to have a moderate to strong impact on stability reducing the percent loss in purity to about 2%, 10%, 6%, 9%, 11%, <1% and 10%, respectively (FIG. 10). The relative ranking was seen to be phosphate>citrate>succinate>other anions such as aspartate, glutamate, acetate, & sulfate, where “>” is used to mean “has greater stabilizing impact than”.

The cationic amino acids lysine, arginine and histidine each had a weak stabilizing effect at 50 mM, reducing the percent loss in purity to about 16% (FIG. 10). The hydrophilic amino acid serine and the hydrophobic amino acids phenylalanine and alanine had weak or even detrimental effect on the stability at 50 mM. Serine and alanine were seen to modestly reduce the percent loss in purity to about 17% and 18%, respectively while phenylalanine increased the percent loss in purity to 26% (FIG. 10A).

Based on the results above, the effect of citrate, arginine and phosphate was also tested at 100 mM, 200 mM and 300 mM concentrations. In these experiments the final antibody concentration was 50 mg/mL in 25 mM histidine buffer, pH 6.0 alone or plus citrate, arginine or phosphate at the indicated concentrations and the samples were incubated for 1 day at 40° C. and the percent loss in purity was determined by SEC (as described above). At 100 mM, citrate and phosphate reduced the percent loss in purity from about 8.4% to ˜1.4% and ˜1.8%, respectively while arginine only reduced the percent loss in purity to 6.0%. At 200 mM and 300 mM citrate and phosphate reduced the percent loss in purity from about 8.4% to ˜0.8% and ˜1.0%, respectively while arginine only reduced the percent loss in purity to ˜4.8% (FIG. 10B).

The chelating agents EDTA and DTPA had little or no effect on the percent loss in purity. Samples containing EDTA or DTPA had a percent loss in purity of about 20% and 24%, respectively, compared to about 22% in the control (10 mM histidine buffer, pH 6.0) (FIG. 10A).

### 8.3.5 Combined Effect of Trehalose and Cationic Amino Acids or Citrate

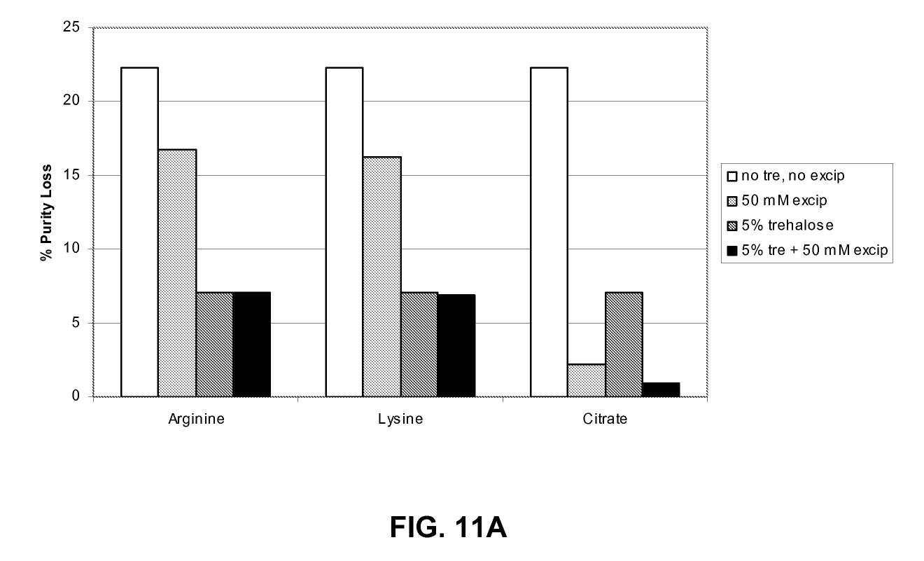

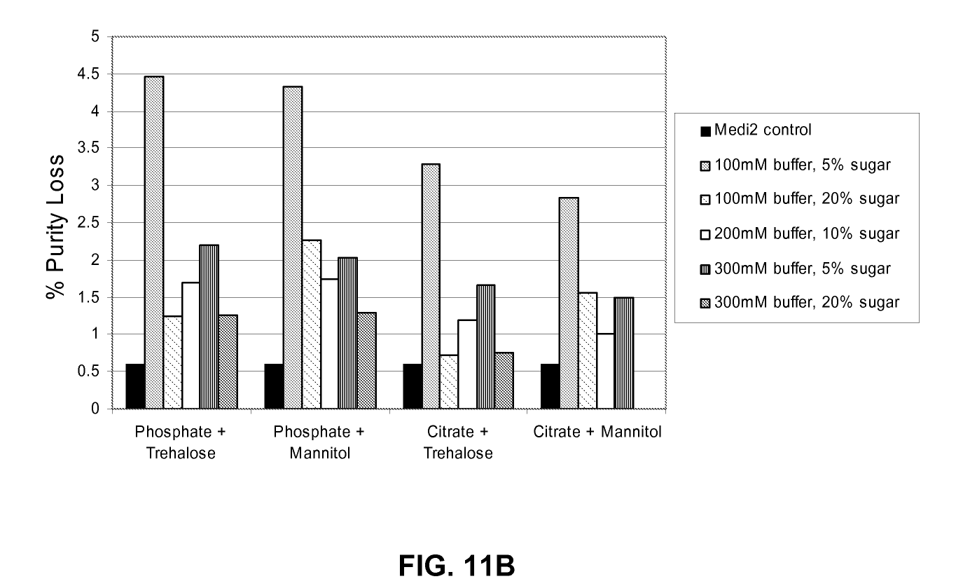

The effect of 50 mM arginine, lysine or citrate was tested alone or in combination with 5% trehalose. The samples were incubated for 19 hours at 40° C. and the percent loss in purity was determined by SEC (as described above). The excipients alone showed similar reductions in the percent loss in purity as compared to the control (10 mM histidine buffer, pH 6.0) as was previously seen (compare FIG. 10A and solid bars in FIG. 11A). The combination of 5% Trehalose with either lysine or arginine at 50 mM had no significant combinatorial effect over Trehalose alone which reduced the percent loss in purity over a 19 hour incubation from about 22% in the control (10 mM histidine buffer, pH 6.0) to about 7% (FIG. 11A). As both lysine and arginine were stabilizing at higher concentrations (see FIG. 8), it is likely that higher concentrations would yield a marked improvement in stability when combined with Trehalose. The combination of 50 mM citrate with 5% Trehalose had a strong combinatorial effect reducing the percent loss in purity to just about 1% compared to ˜7% for Trehalose alone or ˜2% for citrate alone.

The effect of phosphate or citrate in combination with trehalose or mannitol was examined over higher concentration ranges (FIG. 11B). For these experiments the stable wild type Medi2 antibody (50 mg/mL in 10 mM histidine buffer, pH 6.0 with no excipient) was used as a control while Medi3-V3 was formulated to a final concentration of 50 mg/mL in 25 mM histidine buffer, pH 6.0 with 100, 200 or 300 mM phosphate or citrate in combination with 5, 10 or 20% trehalose or mannitol at pH 6.0. The samples were incubated for 1 week at 40° C. and the percent loss in purity was determined by HPLC-SEC as described above. The percent loss in purity for each of the formulations is plotted in FIG. 11B and is summarized in Table 3 below. The loss in purity for the stable control was 0.6%. The 100 mM Citrate, 20% Trehalose; 100 mM Citrate, 20% mannitol and the 300 mM Citrate, 20% Trehalose formulations showed a loss in purity of 1% or less, comparable to that seen for the stable antibody. Several other formulations (e.g., 100 mM phosphate, 20% trehalose; 200 mM phosphate, 10% trehalose; 200 mM phosphate, 10% mannitol; 300 mM phosphate, 20% trehalose; 300 mM phosphate, 20% mannitol; 100 mM citrate, 20% mannitol; 200 mM citrate, 10% trehalose; 300 mM citrate, 5% trehalose and 300 mM citrate, 10% mannitol) showed a loss in purity greater than 1% but less than 2%. The remaining formulations tested all showed a loss of purity of 2% or greater.

TABLE 3
Loss of Purity For Combination Formulations of Phosphate or Citrate and
Trehalose or Mannitol

| | sugar | | | | | |
|---|---|---|---|---|---|---|
| | | 10% | 20% | 5% | 10% | 20% |
| buffer | 5% Trehalose | Trehalose | Trehalose | Mannitol | Mannitol | Mannitol |
| 100 mM | 4.5 | n.t. | 1.2 | 4.3 | n.t. | 2.2 |
| Phosphate | | | | | | |
| 200 mM | n.t. | 1.7 | n.t. | n.t. | 1.7 | n.t. |
| Phosphate | | | | | | |
| 300 mM | 2.2 | n.t. | 1.3 | 2.0 | n.t. | 1.3 |
| Phosphate | | | | | | |
| 100 mM | 3.3 | n.t. | 0.7 | 2.8 | n.t. | 1.6 |
| Citrate | | | | | | |
| 200 mM | n.t. | 1.2 | n.t. | n.t. | 1.0 | n.t. |
| Citrate | | | | | | |
| 300 mM | 1.7 | n.t. | 0.8 | n.t. | 1.5 | n.t. |
| Citrate | | | | | | |

n.t.—not tested

### 8.3.6 Citrate and Histidine as Buffers or Excipients

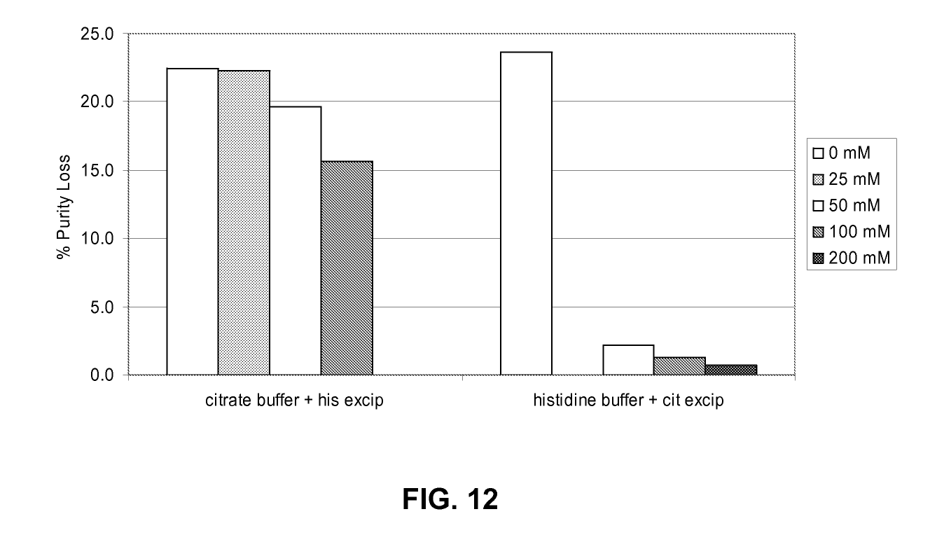

The stabilizing effects of citrate as an excipient and histidine as a buffer were examined as follows, citrate was added, as an excipient, at increasing concentrations (50, 100 and 200 mM) to the stock mAb solution in 10 mM histidine buffer, pH 6.0. Citrate was found to reduce the percent loss in purity over a 19 hour incubation from ˜23% in the control (10 mM histidine buffer, pH 6.0) to ˜2.2%, ˜1.3% and <1% at 50 mM, 100 mM and 200 mM, respectively. To test histidine as an excipient and citrate as a buffer the mAb solution was first dialyzed into a 10 mM Citrate buffer, pH 6.0, and histidine was added at a final concentration of 25, 50 or 100 mM. The stabilizing effect of histidine as an excipient was relatively weak, reducing the percent loss in purity from ˜23% in the control to ˜22%, ˜20% and ˜16% at 25 mM, 50 mM and 100 mM, respectively (FIG. 12). Although either might be used as a buffering agent, citrate has a stronger stabilizing influence as an excipient than histidine. The effect of citrate as a buffer at different concentrations and different pH values was also examined, see below.

### 8.3.7 Effect of pH

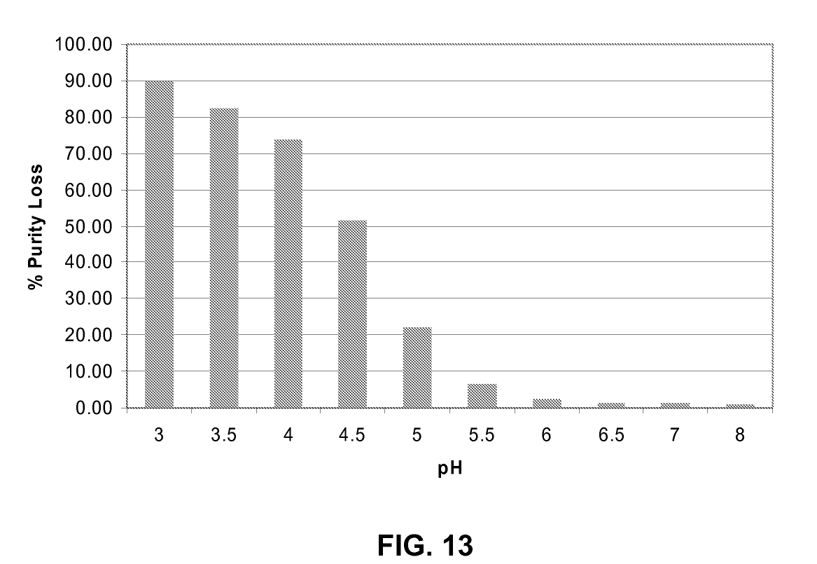

The stabilizing effects of citrate as a buffer over a pH range of 3 to 8 was examined by dialyzing the stock mAb solution into a 50 mM citrate buffer at a pH of between 3 and 8 at half unit increments. The citrate buffered formulations below pH 5.5 showed a percent loss in purity over a 4 hour incubation ranging from a high ˜90% at pH 3 down to ˜21% at pH 5 (FIG. 13). Citrate buffered formulations at pH 5.5 and above showed a percent loss in purity over the same time period of ˜6% at pH 5.5 down to 1% at pH 6.5 and above (FIG. 13). These date indicate that while citrate buffered formulations below pH 5.5 are destabilized those formulations at or above pH 5.5 are stabilized.

### 8.3.8 Citrate Buffer Concentration

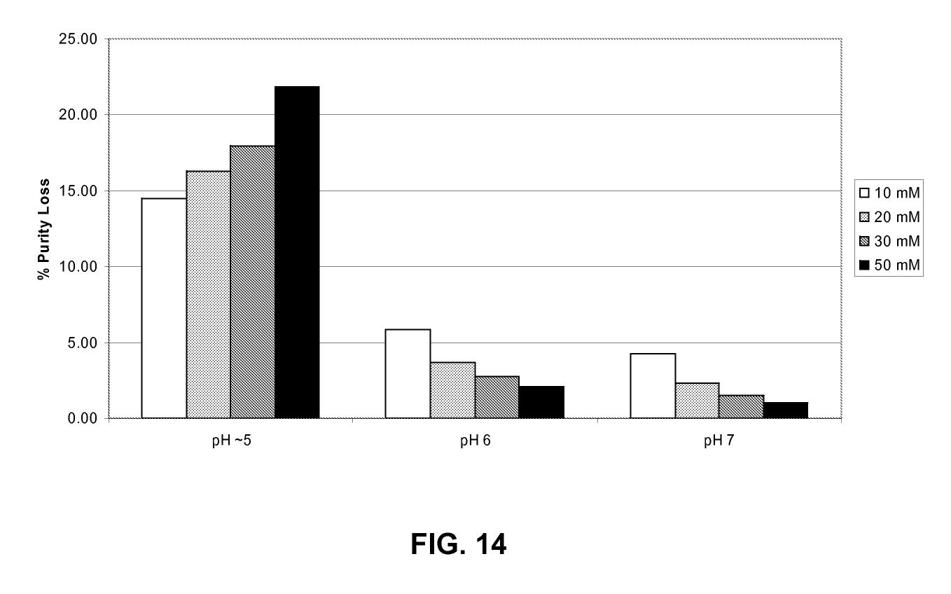

The effect of citrate concentration at pH 5, 6 and 7 was examined by dialyzing the stock mAb solution into a 10 mM, 20 mM, 30 mM or 50 mM citrate buffer each at a pH of 5, 6 and 7. The results plotted in FIG. 14 indicate that at pH 5 higher concentrations of citrate are destabilizing exhibiting a percent loss in purity over a 4 hour incubation of ˜15% to 22% as the concentration of citrate increased. However, at pH 6 and 7 citrate was more stabilizing at all concentrations in the usual buffer range, exhibiting a ˜6% loss in purity at 10 mM, pH 6 and only a ˜1% loss in purity at 50 mM, pH 7. As was seen above, citrate buffered formulations above pH 5.5 are stabilizing. In addition, increasing citrate concentration in the usual buffer concentration range (10-50 mM) is beneficial towards reducing aggregation.

### 8.3.9 Combined Effect of Citrate and Selected Excipients

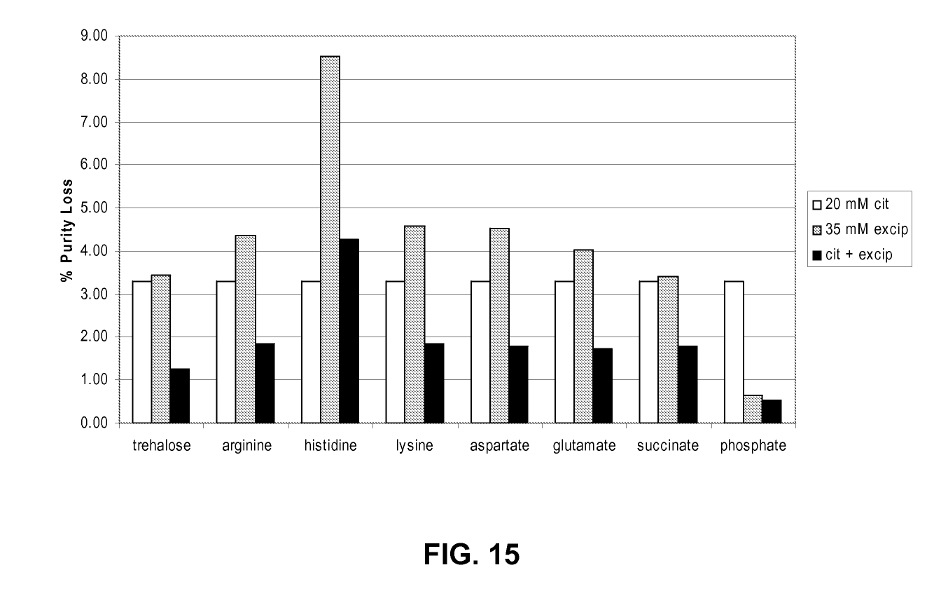

The effect of citrate as an excipient was examined in combination with trehalose, arginine, histidine, lysine, aspartate, glutamate, succinate, or phosphate. For these studies citrate at a final concentration of 20 mM and/or the other excipients at a final concentration of 35 mM were added to the stock mAb solution (10 mM histidine buffer, pH 6.0) as described above. The percent loss in purity over the 4 hour incubation period is plotted in FIG. 15 for each sample. Citrate alone showed about a 3.3% loss while a 3.5%, 4.4%, 8.5%, 4.6%, 4.5%, 4.0% 3.4% and 0.6% loss in purity was seen for trehalose, arginine, histidine, lysine, aspartate, glutamate, succinate and phosphate, respectively. Although the combination of citrate and histidine at these concentrations was somewhat antagonist, resulting in a slight increase in the percent loss in purity over citrate alone (˜3.3% vs. ˜4.3%) the remaining combinations resulted in reduction in the loss of purity over citrate alone and could be considered for formulation. The relative ranking was seen to be phosphate (˜0.5%)>trehalose (˜1.3%)>arginine, histidine, lysine, aspartate, glutamate and succinate (each ˜1.75% to ˜1.85%), where “>” is used to mean “has greater stabilizing impact than”.

### 8.4 Example 4

### DOE Analyzed by Fast Screen Assay

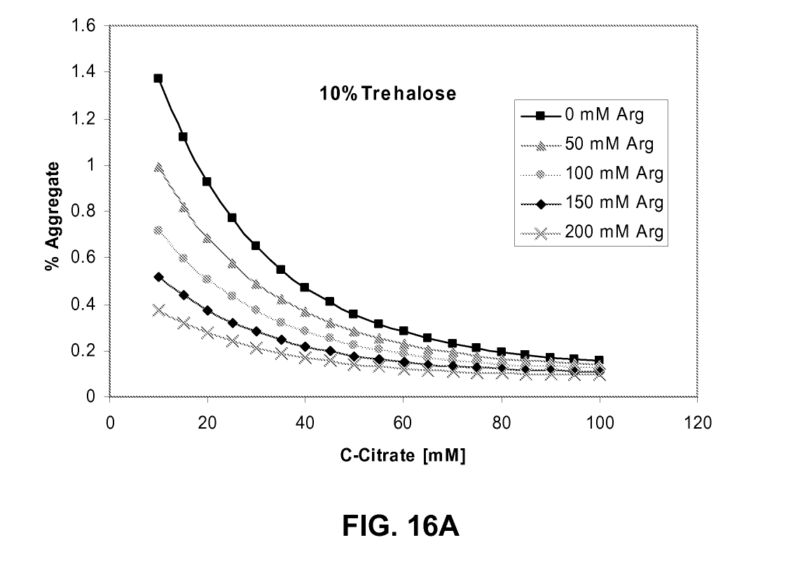

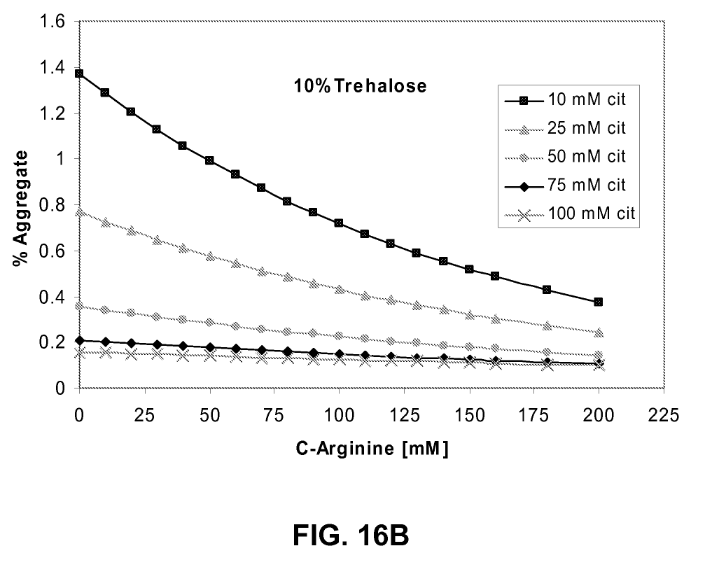

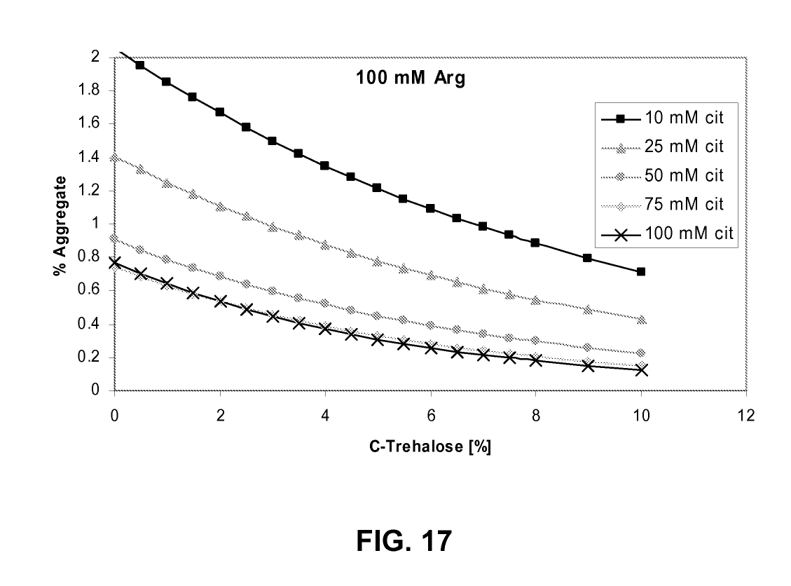

Based on the initial studies Design-Expert (Stat-Ease, Inc., Minneapolis, Minn.) software was used to design a set of experiments to perform exhaustive predictive testing of the combined effects of citrate, arginine and trehalose. 20 solutions were prepared as instructed by Design-Expert, incubated at 40 C for 4 hours, and analyzed by SEC as described above (see Example 1). The results (% Aggregate) were input into Design-Expert, and fitted with a quadratic equation. The theoretical response curves generated using this equation are shown in FIGS. 16-17.

FIGS. 16A and 16B plot the response curves for the effect of different concentrations of citrate and arginine. The effect of citrate is most dramatic when no arginine is present and least dramatic when arginine is present at high concentration. Strong line curvature indicates a high impact on aggregation when increasing citrate concentrations from 10 to 50 mM, but diminishing impact on aggregation when adding citrate at concentrations above 50-60 mM. While increasing arginine concentration is highly beneficial when the citrate concentration is low (<50 mM), but has a minimal effect when the citrate concentration is high (above 50 mM). The response curves for the effect of different concentrations of citrate and trehalose indicate that trehalose has a strong stabilizing effect at all citrate concentrations (FIG. 17).

### 8.5 Example 5

### Formal Stability Assays on Medi3-V3

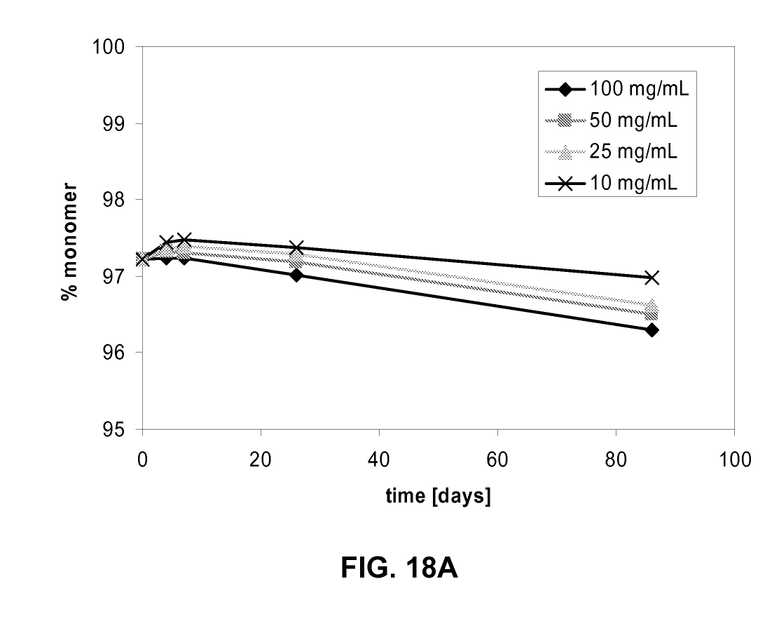

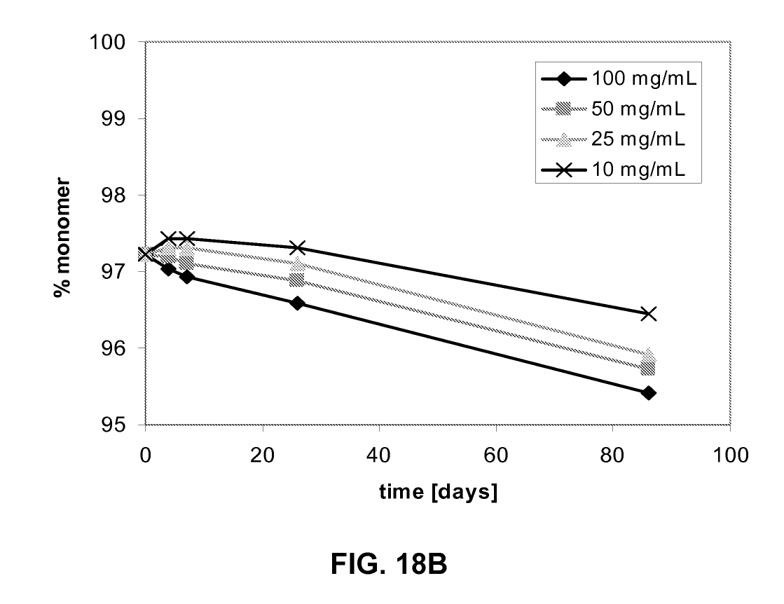

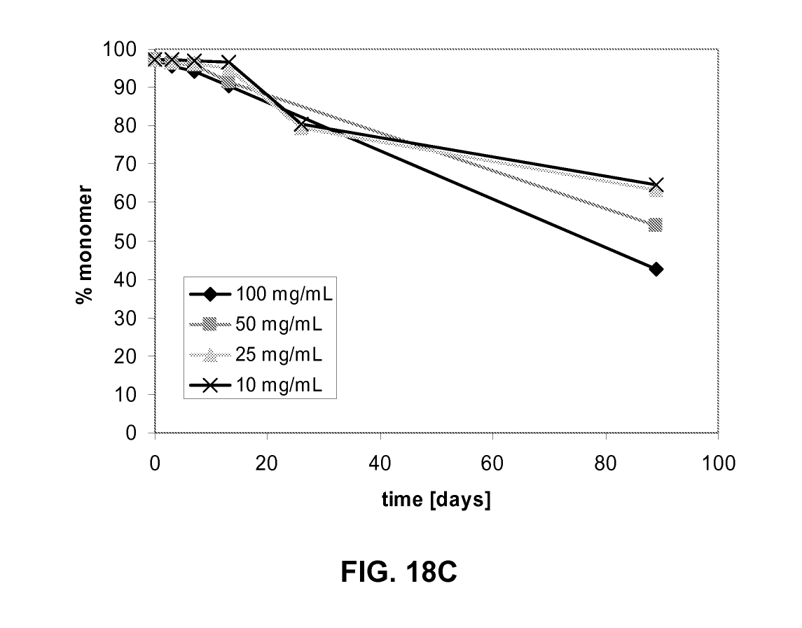

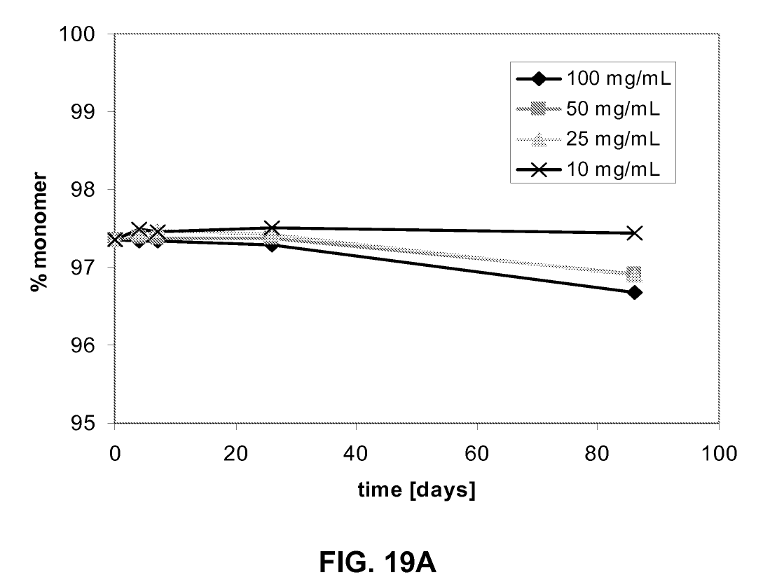

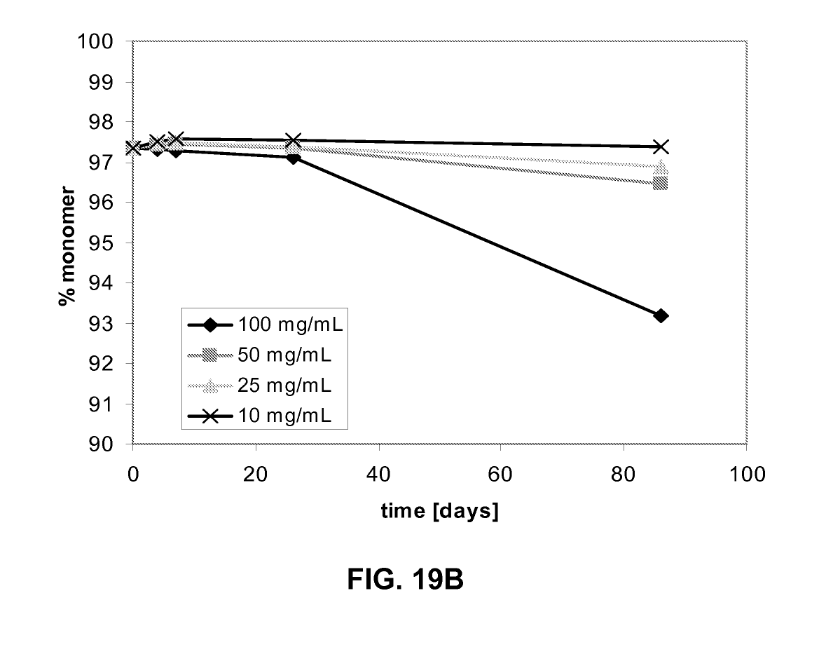

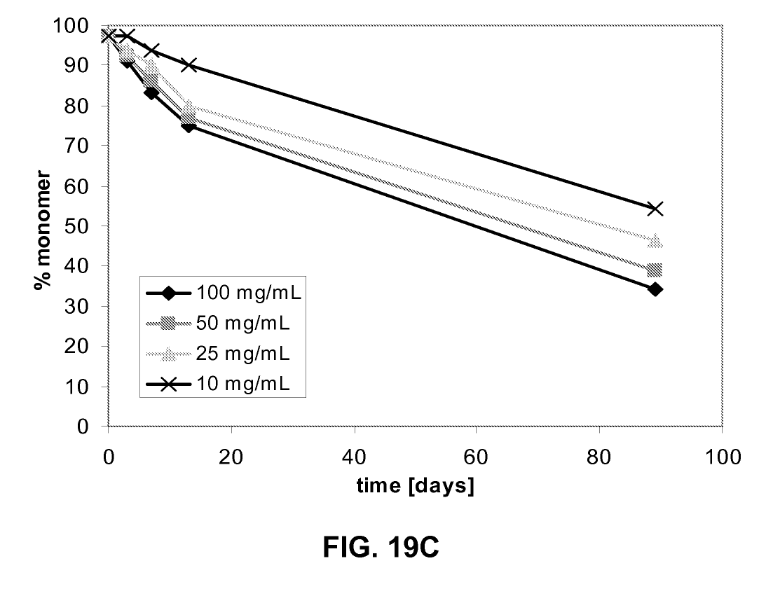

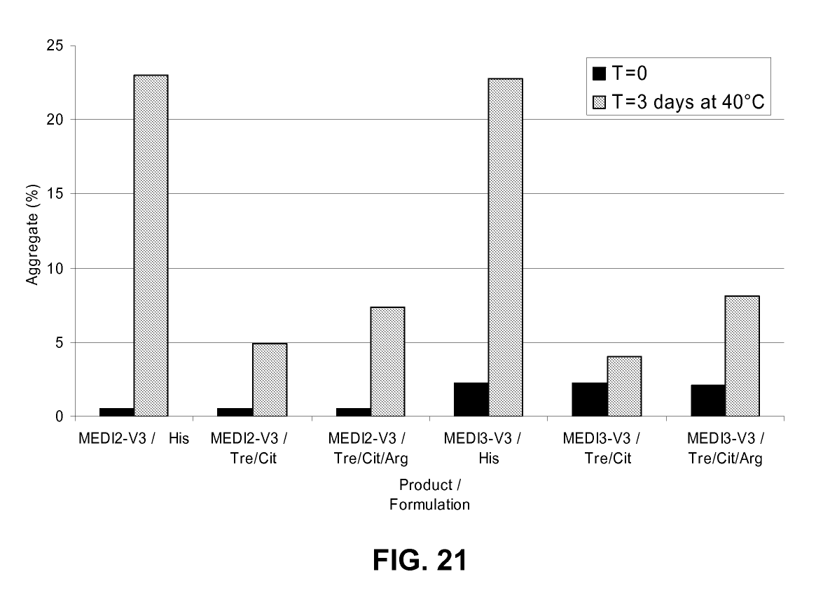

Medi3-V3 was formulated at concentrations of 10, 25, 50 or 100 mg/mL in two specific formulations based on the pilot studies described above and the stability over time at 4° C., 25° C. and 40° C. was monitored by SEC analysis as described above. The percent of monomer present over time for each concentration of Medi3-V3 in Formulation 1 (50 mM citrate, 10% trehalose, pH 6.5) are plotted in FIGS. 18A, B and C (4° C., 25° C. and 40° C., respectively) and for Formulation 2 (25 mM citrate, 200 mM arginine, 8% trehalose, pH 6.5) in FIGS. 19A, B and C (4° C., 25° C. and 40° C., respectively). After 7 days at both 4° C. and 25° C. the percent monomer dropped by less than 1% for each concentration of the antibody in Formulations 1 and 2 (compare FIGS. 18 A-B and 19 A-B). Although at 40° C. the percent monomer dropped by nearly 3% at the highest antibody concentrations in Formulation 1 and by over 14% at the highest antibody concentrations in Formulation 2, these formulations greatly enhanced the stability of Medi3-V3 over the control buffer (10 mM histidine buffer, pH 6.0) which showed nearly a 32% drop in the percent monomer over the same time period (see FIG. 2A). At nearly 90 days the percent monomer dropped by 1% or less for each concentration of antibody in both Formulations 1 and 2 when stored at 4° C. and a drop of 4% or less for each concentration of antibody when stored at 25° C., with Formulation 1 showing slightly better stability at 25° C. than Formulation 2 at the highest antibody concentrations. After nearly 90 days at 40° C. the percent monomer dropped by about 30 to 60% for each concentration in either formulation. However, as described above, both formulations provided significant stabilization over the control buffer at similar incubation times (compare 18C, 19C and FIG. 2A at 7 and 15 days). FIG. 21 plots the percent aggregate present in each formulation at time 0 and after 3 days at 40° C. for Medi3-V3 and for Medi2-V3 (see below for details). These data support the finding that certain formulations are useful to reduce aggregation for any antibody having similar variant Fc regions.

### 8.6 Example 6

### Formal Stability Assays on Medi2-V3

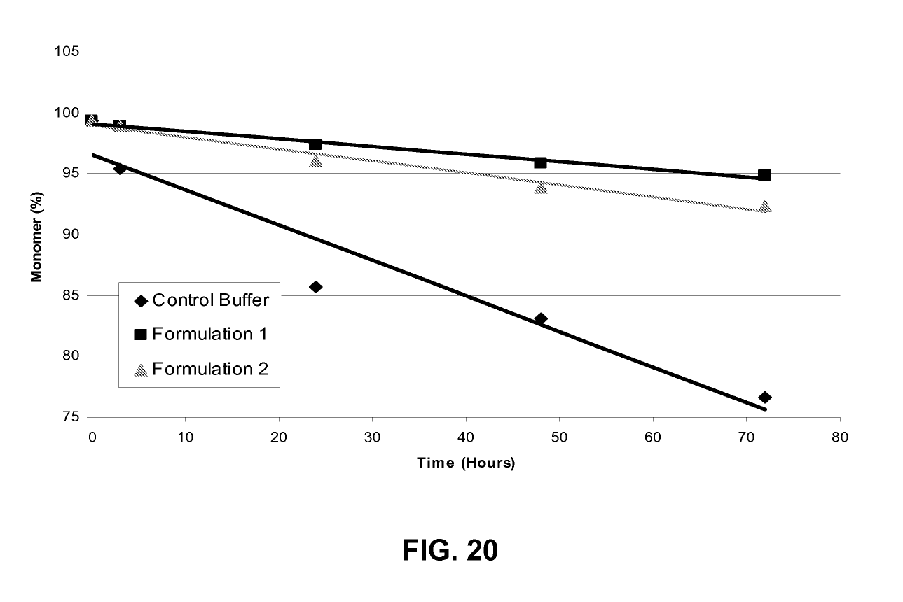

Medi2-V3 was formulated at 80 mg/mL in either control buffer (10 mM histidine buffer, pH 6.0), Formulation 1′ (50 mM citrate, 10% trehalose, pH 6.0) or Formulation 2′ (25 mM citrate, 200 mM arginine, 8% trehalose, pH 6.0) and the stability over a time period of 72 hours at 40° C. was monitored by SEC analysis as described above. The % monomer in the control buffer was seen to drop by about 23.6% over 72 hr, while the drop was just 5% and 7.4% for Formulations 1′ and 2′, respectively (FIG. 20). These data are consistent with those described above and indicate that both Formulations 1′ and 2′ greatly enhanced the stability of Medi2-V3 over the control buffer even at high concentrations (80-100 mg/mL) and high temperatures (25-40° C.). FIG. 21 plots the percent aggregate present in each formulation at time 0 and after 3 days at 40° C. for Medi2-V3 and for Medi3-V3 (see above for details). These data support the finding that certain formulations are useful to reduce aggregation for any antibody having similar variant Fc regions.

### 8.7 Example 7

### Formal Stability Assays on Medi3-V3

To expand on the studies using the anion citrate as a buffer, formulations containing combinations of 100-200 mM citrate and 10-15% trehalose at pH 6.0 or 6.5 were also examined. Medi3-V3 at 100 mg/ml in 100 mM citrate, pH 6.0 was dialyzed into the formulations shown in Table 4 (referred to as formulation A-D) and the volume adjusted to give a final antibody concentration of 50 mg/mL or 100 mg/mL. A formal stability protocol was initiated to monitor long-term stability at 2-8° C., 23-27° C. and 38-42° C. in borosilicate glass vials. The wild type Medi2 antibody formulated at ˜50 mg/mL in 10 mM histidine, pH 6.0 was the control formulation for these studies. The percent aggregate, monomer and fragment were determined by SEC (as described above) at day 0, 7, 15, 21, 28 and 63. In addition changes in charge variants (% prepeak) were determined by IEC at day 0 and day 28.

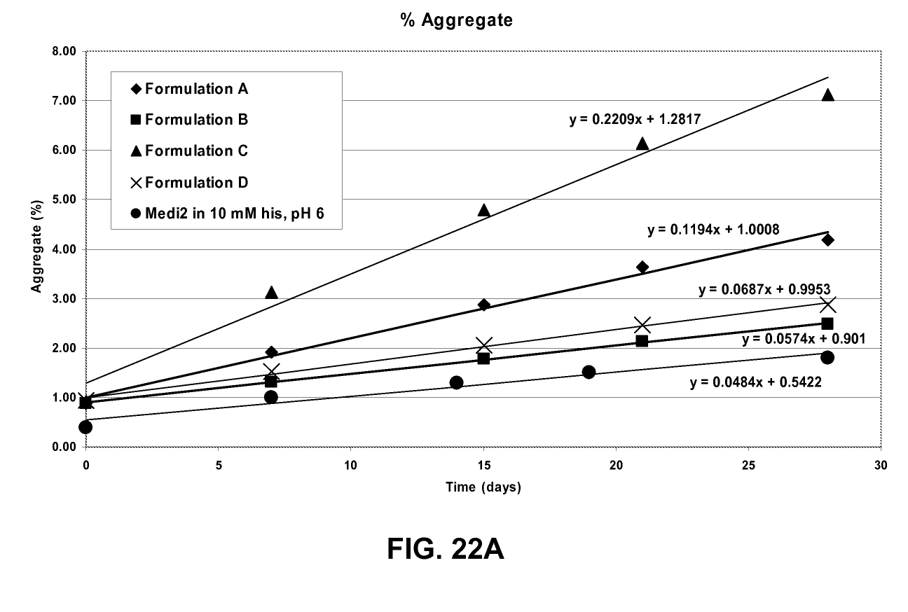

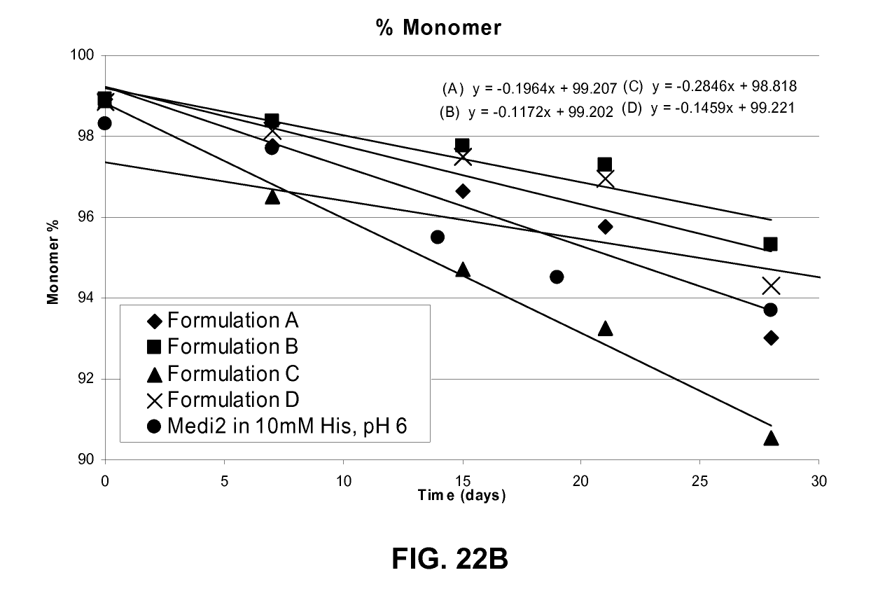

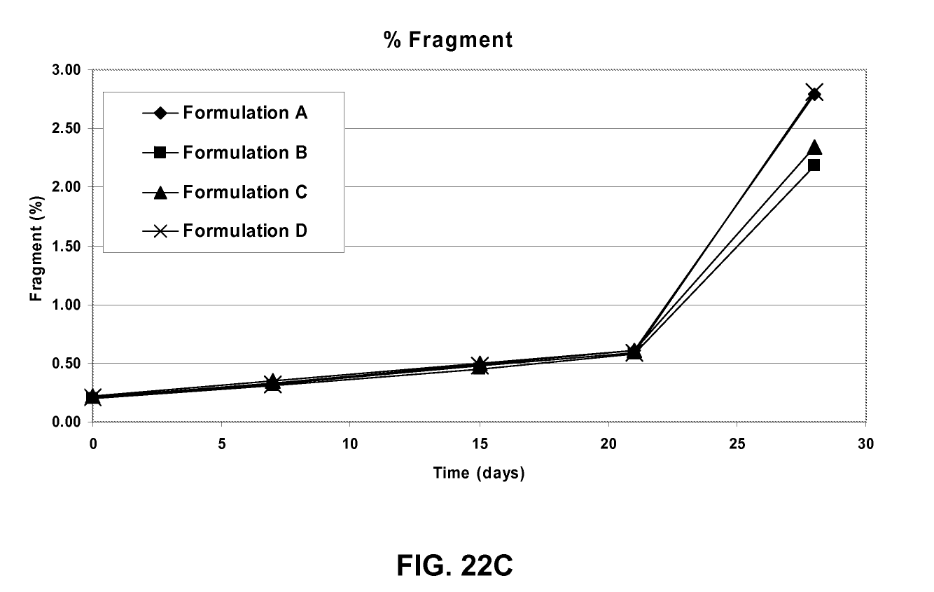

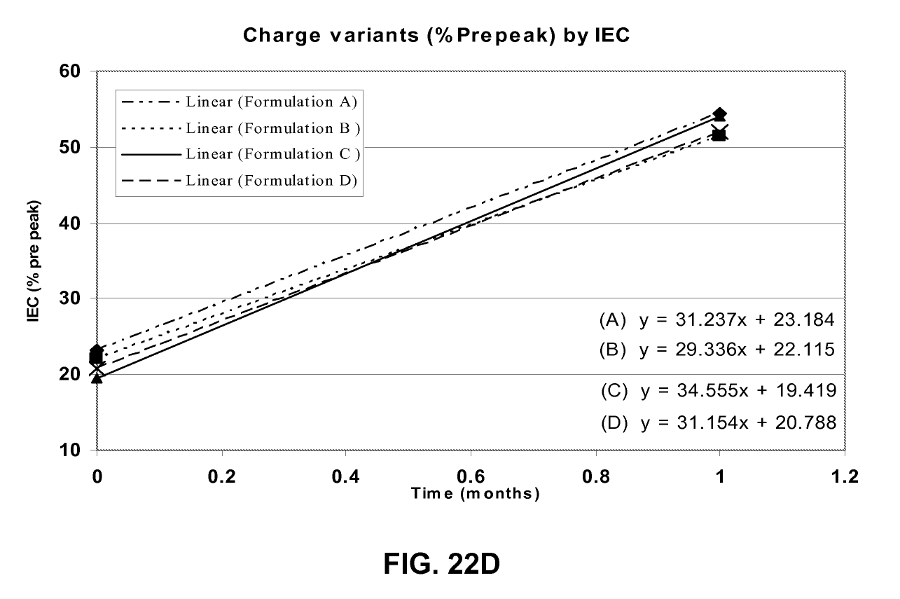

Representative data for the sample held at 38-42° C. for up to 28 days is shown in FIG. 22A-D. The plot of the percent aggregate over time for samples (FIG. 22A) shows that Formulations B and D have an aggregation profile that is very similar to that seen for the stable wildtype antibody with a final % aggregation of just 2.48% and 2.87%, respectively at day 28 compared to 1.8% seen for the control formulation (Table 5). Similarly, Formulations B and D have a % monomer loss of just 3.61% and 4.58%, respectively, compared to 4.6% for the control (FIG. 22B and Table 5). In addition, improved profiles were observed by increasing the buffer pH from 6.0 to 6.5 (compare formulation A and D, Table 5). This improvement is even more pronounced when the concentration of the antibody is taken into account as the concentration of MedI3-V3 is 50 mg/ml in the less stable formulation A (pH 6.0) and 100 mg/ml in the more stable formulation D (pH 6.5) and earlier studies showed that the aggregation of MedI3-V3 increases with antibody concentration (see, e.g., FIG. 4). At the 50 mg/mL concentration Formulation B performed better than Formulation A, while at 100 mg/mL Formulation D performed the best. These trends were also observed for the samples at 63 days (Table 5 and data not shown). No difference in the fragment levels was seen between Formulations A-D (FIG. 22C).

Interestingly, all of the formulations had better stability than the control at both 23-27° C. and 2-8° C. (Table 5 and data not shown) suggesting that these formulations may stabilize wild type antibodies more effectively. At these temperatures the profiles of Formulations A, B were nearly identical with aggregation rates of less than 0.01 at 2-8° C. and less than 0.1 at 23-27° C. In contrast to what was observed at 38-42° C., Formulation C performed better than Formulation D.

As deamidation may be accelerated at higher pH, IEC was used to monitor any change in the charge of the antibodies in Formulations A-D at 40° C. The pre-peak levels are indicative of charge variant changes due to processes such as deamidation. As shown in FIG. 22D, there is no difference in the levels of charge variants generated by incubation at 40° C. in Formulations A-D indicating that increasing the pH from 6.0 to 6.5 does not induce the production of more charge variants.

These data indicate that V3-like antibodies can be effectively stabilized in Formulation A (100 mM Citrate, 15% Trehalose, pH 6.0) over a broad range of temperatures (2-42° C.). At higher temperatures (38-42° C.) Formulations A and D were comparable, while at lower temperatures (2-27° C.) Formulations A and B were comparable. These data also indicate that formulations comprising higher concentrations of trehalose (>10%) and higher pH (>6.0) improve the stability profile of MedI3-V3, and antibodies having similar variant Fc regions at higher temperatures. This study along with the data presented in FIG. 14 indicate that a pH of at least 6.5 to 7.0 or even higher could improve the aggregation profile and accordingly, the overall stability of V3-like antibodies at higher temperatures. While at lower temperatures citrate and lower pH (˜6.0) offer improved stability.

TABLE 4
Formulation Used in Medi-V3 Formal Stability Assays

| | | | | | |
|---|---|---|---|---|---|
| | Conc. | | | | |
| mAb | mg/mL | Formulation | Citrate | Tre. | pH |

| | | | | |
|---|---|---|---|---|
| MedI2 | 50 | Y | 10 mM Histidine | 6.0 |

| | | | | | |
|---|---|---|---|---|---|
| MedI3- | 50 | A | 200 mM | 10% | 6.0 |
| V3 | 50 | B | 100 mM | 15% | 6.0 |
| | 100 | C | 200 mM | 10% | 6.0 |
| | 100 | D | 200 mM | 10% | 6.5 |

TABLE 5
Incubation Results

| | | | | | | |
|---|---|---|---|---|---|---|
| | | Aggregation | % AggregateGain @ | % Monomer | % Aggregate | % Monomer |
| mAb | Formulation | Rate 2 month^{≠} | 1 month^{≠} | Loss @ 1 month^{≠} | Gain @ 2 month^{≠} | Loss @ 2 month^{≠} |

38-42° C.

| | | | | | | |
|---|---|---|---|---|---|---|
| MedI2 | Y | 1.0024 | 1.8 | 4.60 | 2.3 | 6.8 |
| MedI3-V3 | A | 3.7512 | 4.18 | 5.90 | 7.66 | 11.33 |
| | B | 2.1934 | 2.48 | 3.61 | 4.41 | 8.02 |
| | C | 5.9707 | 6.14 | 8.37 | 12.36 | 16.03 |
| | D | 2.4236 | 2.87 | 4.58 | 4.92 | 8.68 |

23-27° C.

| | | | | | | |
|---|---|---|---|---|---|---|
| MedI2 | Y | 0.3338 | 0.3 | 0.4 | 0.7 | 2.9* |
| MedI3-V3 | A | 0.078 | 0.04 | 0.10 | 0.16 | 0.29 |
| | B | 0.0812 | 0.04 | 0.13 | 0.16 | 0.29 |
| | C | 0.1363 | 0.12 | 0.19 | 0.27 | 0.39 |
| | D | 0.2031 | 0.2 | 0.25 | 0.41 | 0.52 |

2-8° C.

| | | | | | | |
|---|---|---|---|---|---|---|
| MedI2 | Y | 0.1888 | 0.3 | 0.2 | 0.4 | 0.3 |
| MedI3-V3 | A | −0.0027 | −0.04 | −0.03 | −0.01 | −0.01 |
| | B | 0.009 | −0.04 | −0.02 | 0.02 | 0.02 |
| | C | 0.0165 | 0.00 | 0.00 | 0.03 | 0.02 |
| | D | 0.0434 | 0.03 | 0.04 | 0.09 | 0.08 |

^{≠}In these studies the 1 month values were determined at day 28 and 2 month values were determined at day 63.
*integration difficulties.

### 8.7.1Methods

Ion Exchange Chromatography: The column was a ProPac WCX-10 4×250 mm Analytical Column, Dionex Cat# 54993. The buffers were: A—20 mM sodium phosphate, pH 7.0 and B—20 mM sodium phosphate, 100 mM sodium chloride, pH 7.0. Samples were prepared by diluting to 3 μg/μL in buffer A, and 25 μL of each sample was infected, giving 75 μg of injected sample. The elution gradient was as follows:

| | |
|---|---|
| Time | % Buffer B |

| | | |
|---|---|---|
| 0 | min | 20% |
| 5 | min | 30% |
| 45 | min | 60% |
| 45.1 | min | 90% |
| 50 | min | 20% |

Peaks typically eluted between 10-40 minutes. Protein elution was monitored by absorbance at 220 nm.

### 8.8 Example 8

### Melting Temperature Analysis in Different Buffers

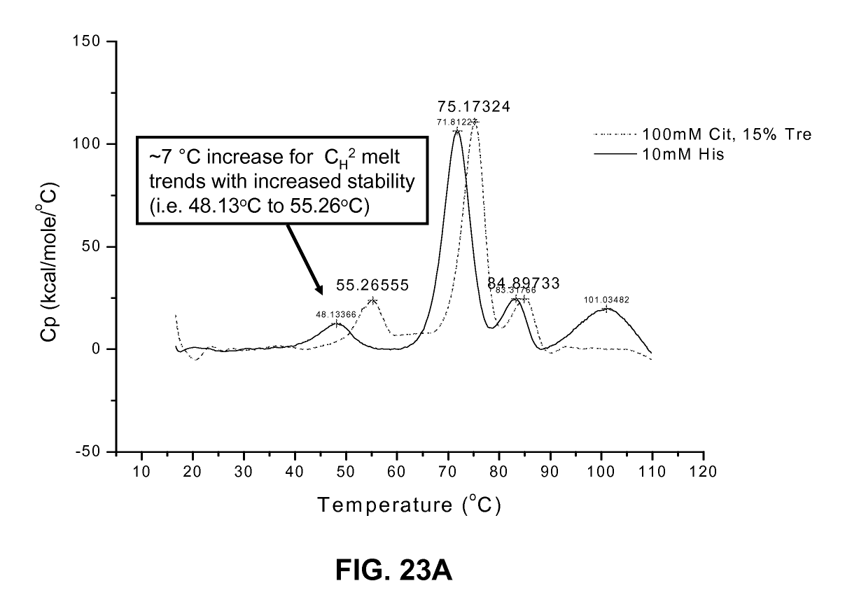

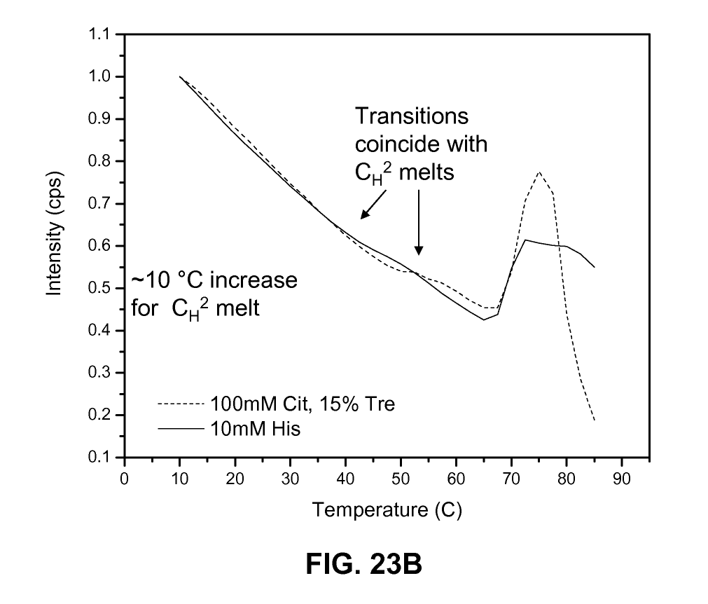

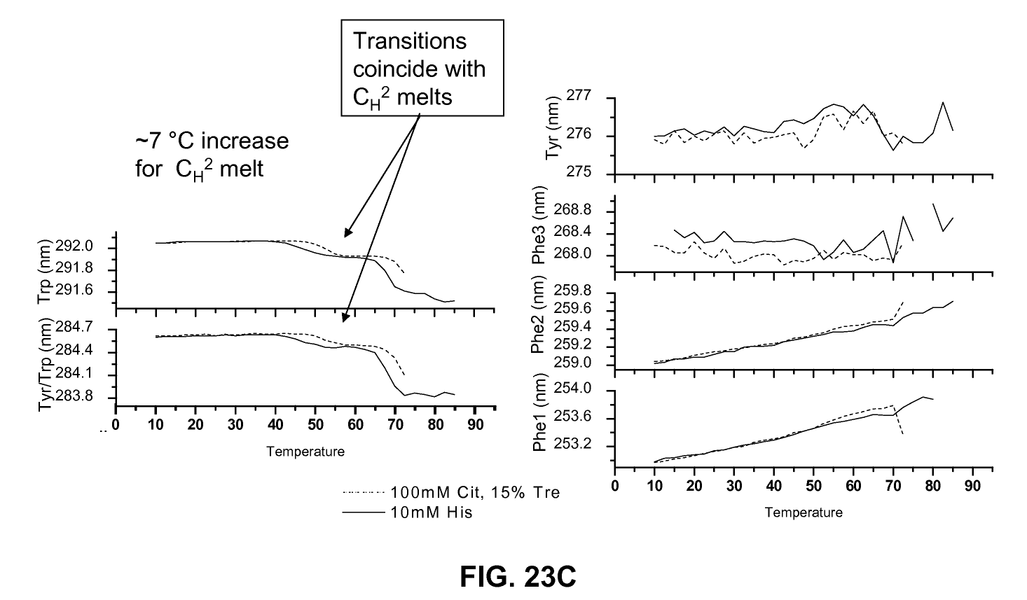

As demonstrated above (Example 1) the melting temperature of the C_{H}² domain is lower in the V3-like antibodies. To examine what effect the formulation has on the Tm of a V3-like antibody the Tm of Medi3-V3 (0.5 mg/mL) formulated in 10 mM Histidine, pH 6 or in 100 mM Citrate, 15% Trehalose (Formulation B) by DSC as described above. As shown in FIG. 23A, the Tm of the C_{H}² domain increased from ˜48° C. to ˜55° C. when Medi3-V3 was tested in Formulation B. Similarly, an ˜10° C. increase for C_{H}² domain melting, was also observed by Fluorescence (FIG. 23B) and a 7° C. increase was observed by 2nd Derivative UV-Vis. monitored melting (FIG. 23C) of Medi3-V3 in Formulation B. These data indicate that Formulation B, and likely the other stabilizing Formulations, act at least in part by increasing the Tm of the C_{H}² domain. Thus, these formulations likely have broad applicability in stabilizing antibodies with variant Fc regions having lower melting temperatures. In addition, these studies indicate that DSC is a useful tool to use to examine the potential of a formulation to stabilize antibodies in general and V3-like antibodies in particular.

### 8.8.1 Methods

Sample preparation: Medi3-V3 was reconstituted in WFI to give a concentration of ˜40 mg/mL. The formulation was subsequently dialyzed into 10 mM His, pH 6 utilizing 3,500 MWCO Pierce dialysis cassettes. After a 1-L exchange which lasted>8 hours, the sample was tested for pH and osmolality to confirm complete buffer exchange. The sample was then either diluted to ˜0.5 mg/mL into either 10 mM His, pH 6 or 100 mM Citrate, 15% Tre, pH 6 for further biophysical studies (DSC, fluorescence, and UV spectroscopy).

Fluorescence monitored melting: Fluorescence emission spectra were collected with a QuantaMaster™ fluorometer (Photon Technologies Incorporated Monmouth, N.J., 75W Xenon arc lamp, Model 810 pmt detector, and FeliX32 v. 1.0 operating software). The tryptophan emission spectrum was collected from 305-450 nm upon excitation at 295 nm. Emission spectra were collected over the temperature range of 10-85° C. after holding the 0.5 mg/mL MEDI-531 sample at each temperature for 5 minutes. Relative emission intensity was calculated by dividing the intensity collected at 329 nm at the tested temperature by the value collected at the same wavelength and 10° C.

2nd Derivative UV-Vis. monitored melting: An Agilent 8453 diode-array UV-Visible spectrophotometer (Palo Alto, Calif.) was employed for UV absorbance studies. The temperature was increased from 10-85° C. while incubating 0.5 mg/mL MEDI-531 solutions in a 1-cm path length cell at each temperature for 5 minutes before collecting a 25-second absorbance spectrum. The spectra were analyzed for shifts in the second derivative peak positions of the aromatic amino acids with increasing temperature. Second derivative spectra were obtained through calculation based upon a fifth degree Savitzky-Golay polynomial, 9-data point filter length window and fit to a cubic function. Finally, 0.01 nm resolved 2^{nd} derivative spectra were obtained by splining using 99 interpolated points between each one-nanometer data point. The resulting 2^{nd} derivative spectrum had theoretical peak resolutions of approximately 0.01 nm. The negative peak positions of the aromatic residues were monitored between 250 and 300 nm for indications of changes of tertiary structure with increasing temperature.

### 8.9 Example 9

### Excipients which can Increase Aggregation

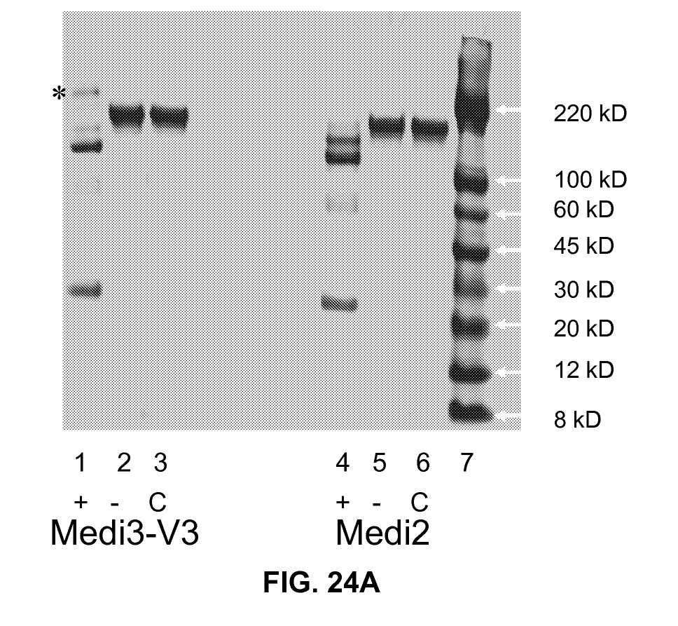

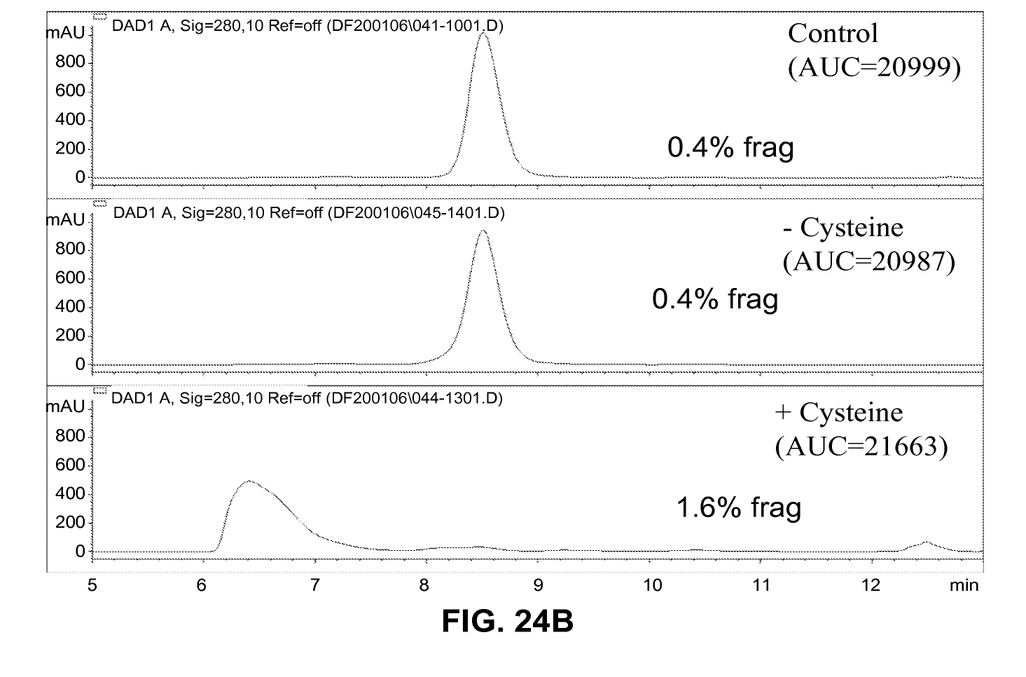

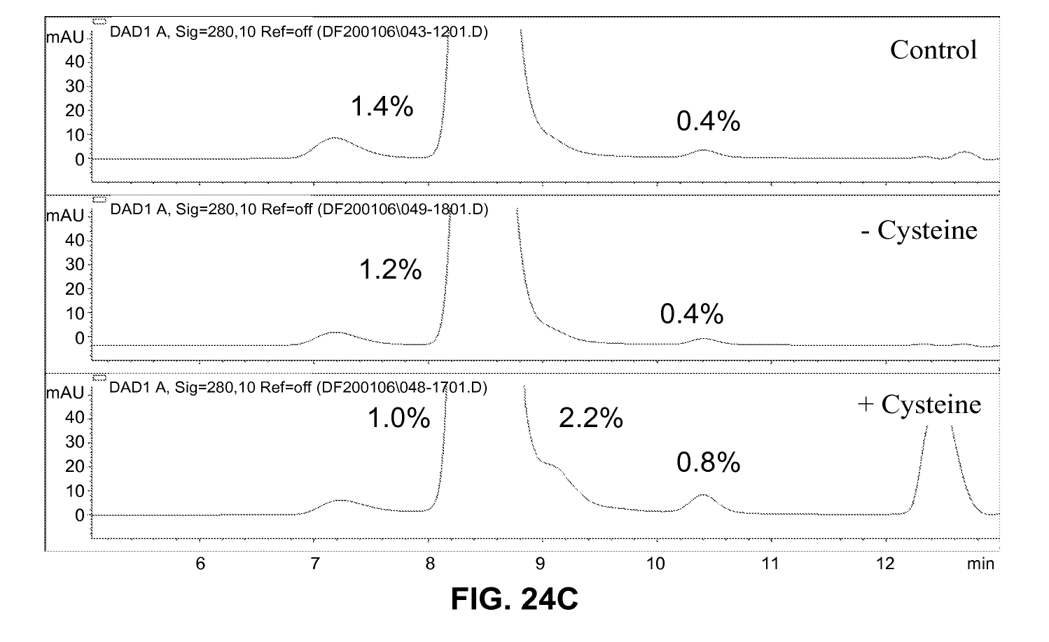

As described above cysteine was seen to increase aggregation. To further characterize this effect Medi3-V3 and Medi2 (having a wild type Fc region) were incubated in the presence or absence of 50 mM cysteine for 16 hours at 37° C. and analyzed by non-reducing SDS-PAGE without heating and by SEC. A control sample which was not incubated at 37° C. was also analyzed. Both antibody samples incubated with cysteine dissociate into separate heavy and light chains when run on SDS-PAGE (FIG. 24A, lanes 1 and 4). In addition a small amount of fragmentation is present in each +cysteine sample and the Medi3-V3+cysteine appears to have a small amount of covalent aggregate present (FIG. 24A, asterisk). The same samples were also analyzed by SEC (FIG. 24B-C). The Medi3-V3 control sample and −cysteine samples showed little aggregation (FIG. 22B, top and middle) while the +cysteine sample ran almost entirely as an aggregate with little monomer present (FIG. 24B, bottom). In contrast, the Medi2 samples showed a constant low amount of aggregate (˜1 to 1.4%) which did not increase in the +cysteine sample (FIG. 24C, compare all three profiles). Both antibodies showed a slight increase in fragments after incubation with cysteine with Medi3-V3 increasing from 0.4% to 1.6% and Medi2 increasing from 0.4% to 0.8%. Together these data indicate that cysteine increases the formation of non-covalent aggregates and may increase fragmentation of antibodies having the V3 Fc region but not those having a wild type Fc region.

### 8.10 Example 10

### Stability of Lyophilized Formulations of Medi3-V3

Medi3-V3 was formulated in one of the formulations shown in Table 6. For these runs the pre-lyophilization bulk was formulated to allow for reconstitution in one of the following:

i) in 1 mL of WFI to one half the volume of the bulk fill such that the final concentrations of all components is roughly 200% of the original concentrations in the bulk liquid drug substance.

ii) in 3 mL of WFI so that the final concentrations of all components is roughly 75% of the original concentrations in the bulk liquid drug substance.

TABLE 6
Pre-Lyophilization Bulk Formulations

| | C | | | Ionic | | |
|---|---|---|---|---|---|---|
| Formulation | [mg/mL] | Buffer | Sugar | Stabilizer | Surfactant | pH |
| #1 | 50 | 10 mM | 6% Trehalose | 2% arginine | 0.025% PS-80 | 6.0 |
| | | histidine | | | | |
| #2 | 20 | 10 mM | 6% Trehalose | 2% arginine | 0.025% PS-80 | 6.0 |
| | | histidine | | | | |
| #3 | 50 | 10 mM | 6% Trehalose | 2% arginine | 0.025% PS-80 | 6.0 |
| | | histidine | | | | |
| CL | 50 | 25 mM | 6% Trehalose | 2% lysine | 0.025% PS-80 | 6.0 |
| | | citrate | | | | |
| A | 50-56 | 10 mM | 6% Trehalose | 2% arginine | 0.025% PS-80 | 6.0 |
| | | histidine | | | | |
| HL | 50-56 | 10 mM | 6% Trehalose | 2% lysine | 0.025% PS-80 | 6.0 |
| | | histidine | | | | |

The samples were lyophilized under the conditions described below. The lyophilized samples were stored at 2-8° C., 23-27° C., and 38-42° C. and tested at timepoints corresponding to the ICH guidelines. There was minimal or no loss in purity upon reconstitution (see Table 7) after lyophilization. After incubation at 38-42° C. (referenced as 40° C.) there was minimal purity loss compared to the liquid formulations (Table 7), indicating that the pre-lyophilization formulations were also stabilizing in the solid state.

TABLE 7
Stability of Post Lyophilized Formulations

| | Pre-lyo | | | Reconstitution | | | | | |
|---|---|---|---|---|---|---|---|---|---|
| | Bulk | Fill | | Characteristics | | | | Purity Loss | |
| | Medi3-V3 | Vol | Dose | WFI Vol | Time | Medi3-V3 | Osmol | Pre to | 12 days |
| Sample # | mg/mL | (mL) | (mg) | (mL) | (min) | mg/mL | (mOsm) | Post Lyo | at 40° C. |
| 1 | 50 | 2.2 | 100 | 1 | >5 | 80-100 | 846 | 0 | 0.2 |
| 2 | 20 | 2.2 | 40 | 1 | >5 | 30-50 | 747 | 0 | 0 |
| 3 | 50 | 2.2 | 100 | 1 | >5 | 80-100 | 610 | 0 | 0.4 |
| CL | 50 | 2.2 | 100 | 1 | >5 | 80-100 | NS | 0 | 0.9 in 1 mo |
| A | 50 | 2.4 | 100 | 3 | <2 | 40 | 281 | 0.1 | 1.5 in 1 yr |
| HL | 50 | 2.4 | 100 | 3 | <2 | 40 | NS | 0.1 | 1.9 in 1 yr |

Lyophilization: formulated Medi3-V3 was filled at 2.3-2.4 mL in a biosafety cabinet into 5 cc vials and partially stoppered. The vials were placed in a hexagonal-close-packed configuration on a tray and transferred onto the shelf of a Virtis Genesis lyophilizer. The lyophilization cycle consisted of the following steps:

1) Start with vials at 5-20° C., atmospheric pressure,

2) a “freeze-step” temperature ramp to −40° C. at 0.5° C./min,

3) a 120 min hold at 40° C. during which time the vacuum was dropped to 125 mTorr and the condensor temperature was dropped to −60° C.,

4) ramp temperature to between −10° C. and −20° C., hold at that temp for the duration of the primary dry (44 hours), and

5) a secondary dry at 25° C. for 10-18 hours. Then the lyophilizer was backfilled with dry nitrogen gas to a pressure of 600-700 Torr, and the vials were stoppered using the hydraulic system of the lyophilizer. Next the chamber was vented to atmosphere and the vials were removed.

Whereas, particular embodiments of the invention have been described above for purposes of description, it will be appreciated by those skilled in the art that numerous variations of the details may be made without departing from the invention as described in the appended claims.

All publications, patents and patent applications mentioned in this specification are herein incorporated by reference into the specification to the same extent as if each individual publication, patent or patent application was specifically and individually indicated to be incorporated herein by reference.

1. A liquid formulation comprising an Fc variant protein, a buffering agent at a concentration between 1 mM to 100 mM and further comprising one or more component selected from the group consisting of:
(a) a carbohydrate excipient at a concentration between 1% to 20% weight to volume;
(b) a cationic amino acid at a concentration between 1 mM to 400 mM;
(c) an anion at a concentration between 1 mM to 200 mM; and
(d) a polysorbate at a concentration between 0.001% to 0.1%,
wherein, said formulation has a pH of about 5.5 to about 8.

2. The liquid formulation of claim 1, comprising component (a), (b) and optionally (d).

3. The formulation of claim 1, comprising component (a), (c) and optionally (d).

4. The liquid formulation of claim 1, wherein the Fc variant protein has at least 10% less aggregation when compared to the aggregation when the same Fc variant is formulated in 10 mM Histidine pH 6.0.

5. The liquid formulation of claim 1, wherein the Fc variant protein is an antibody or an Fc fusion protein.

6. The liquid formulation of claim 1, wherein the buffering agent is histidine, phosphate or citrate.

7. The liquid formulation of any of claim 1, wherein the carbohydrate excipient is trehalose, sucrose, mannitol, maltose, orraffinose.

8. The liquid formulation of any of claim 1, wherein the cationic amino acid is lysine, arginine or histidine.

9. The liquid formulation of claim 1, wherein the anion is citrate, succinate or phosphate.

10. The liquid formulation of claim 1, wherein the pH is between 6.0 and 6.5.

11. The liquid formulation of claim 1, wherein the Fc variant protein competes for binding to the same antigen as a clinical product or candidate antibody selected from the group consisting of: rituximab, zanolimumab, hA20, AME-133, HumaLYM, trastuzumab, pertuzumab, cetuximab, IMC-3G3, panitumumab, zalutumumab, nimotuzumab, matuzumab, ch806, KSB-102, MR1-1, SC100, SC101, SC103, alemtuzumab, muromonab-CD3, OKT4A, ibritumomab, gemtuzumab, alefacept, abciximab, basiliximab, palivizumab, motavizumab, infliximab, adalimumab, CDP-571, etanercept, ABX-CBL, ABX-IL8, ABX-MA1 pemtumomab, Therex, AS1405, natalizumab, HuBC-1, natalizumab, IDEC-131, VLA-1; CAT-152; J695, CAT-192, CAT-213, BR3-Fc, LymphoStat-B, TRAIL-R1mAb , bevacizumab, ranibizumab, omalizumab, efalizumab, MLN-02, zanolimumab, HuMax-IL 15, HuMax-Inflam, HuMax-Cancer, HuMax-Lymphoma, HuMax-TAC, clenoliximab, lumiliximab, BEC2, IMC-1C11, DC101, labetuzumab, arcitumomab, epratuzumab, tacatuzumab, MyelomaCide, LkoCide, ProstaCide, ipilimumab, MDX-060, MDX-070, MDX-018, MDX-1106, MDX-1103, MDX-1333, MDX-214, MDX-1100, MDX-CD4, MDX-1388, MDX-066, MDX-1307, HGS-TR2J, FG-3019, BMS-66513, SGN-30, SGN-40, tocilizumab, CS-1008, IDM-1, golimumab, CNTO 1275, CNTO 95, CNTO 328, mepolizumab, MOR101, MOR102, MOR201, visilizumab, HuZAF, volocixmab, ING-1, MLN2201, daclizumab, HCD122, CDP860, PRO542, C14, oregovomab, edrecolomab, etaracizumab, siplizumab, lintuzumab, Hu1D10, Lym-1, efalizumab, ICM3, galiximab, eculizumab, pexelizumab, LDP-01, huA33, WX-G250, sibrotuzumab, Chimeric KW-2871, hu3S193, huLK26; bivatuzumab, ch14.18, 3F8, BC8, huHMFG1, MORAb-003, MORAb-004, MORAb-009, denosumab, PRO-140, 1D09C3, huMikbeta-1, NI-0401, NI-501, cantuzumab, HuN901, 8H9, chTNT-1/B, bavituximab, huJ591, HeFi-1, Pentacea, abagovomab, tositumomab, 105AD7, GMA161 and GMA321.

12. The liquid formulation of claim 1, wherein the Fc variant protein comprises an Fc region with enhanced ADCC activity relative to a protein having the same amino acid sequence except having a naturally occurring Fc region.

13. The liquid formulation of claim 12, wherein the Fc variant protein comprises an Fc region having a non naturally occurring amino acid residue at one or more positions selected from the group consisting of: 234, 235, 236, 239, 240, 241, 243, 244, 245, 247, 252, 254, 256, 262, 263, 264, 265, 266, 267, 269, 296, 297, 298, 299, 313, 325, 326, 327, 328, 329, 330, 332, 333, and 334 as numbered by the EU index as set forth in Kabat.

14. The liquid formulation of claims 12, wherein the Fc variant protein comprises an Fc region having at least one non naturally occurring amino acid residue selected from the group consisting of: 234D, 234E, 234N, 234Q, 234T, 234H, 234Y, 234I, 234V, 234F, 235A, 235D, 235R, 235W, 235P, 235S, 235N, 235Q, 235T, 235H, 235Y, 235I, 235V, 235F, 236E, 239D, 239E, 239N, 239Q, 239F, 239T, 239H, 239Y, 240I, 240A, 240T, 240M, 241W, 241L, 241Y, 241E, 241R, 243W, 243L 243Y, 243R, 243Q, 244H, 245A, 247V, 247G, 252Y, 254T, 256E, 262I, 262A, 262T, 262E, 263I, 263A, 263T, 263M, 264L, 264I, 264W, 264T, 264R, 264F, 264M, 264Y, 264E, 265G, 265N, 265Q, 265Y, 265F, 265V, 265I, 265L, 265H, 265T, 266I, 266A, 266T, 266M, 267Q, 267L, 269H, 269Y, 269F, 269R, 296E, 296Q, 296D, 296N, 296S, 296T, 296L, 296I, 296H, 269G, 297S, 297D, 297E, 298H, 298I, 298T, 298F, 299I, 299L, 299A, 299S, 299V, 299H, 299F, 299E, 313F, 325Q, 325L, 325I, 325D, 325E, 325A, 325T, 325V, 325H, 327G, 327W, 327N, 327L, 328S, 328M, 328D, 328E, 328N, 328Q, 328F, 328I, 328V, 328T, 328H, 328A, 329F, 329H, 329Q, 330K, 330G, 330T, 330C, 330L, 330Y, 330V, 330I, 330F, 330R, 330H, 332D, 332S, 332W, 332F, 332E, 332N, 332Q, 332T, 332H, 332Y, and 332A as numbered by the EU index as set forth in Kabat.

15. The liquid formulation of claim 13, wherein the Fc region comprises a non naturally occurring amino acid at one or more positions selected from the group consisting of 239, 330 and 332, as numbered by the EU index as set forth in Kabat.

16. The liquid formulation of claim 14, wherein the at least one non naturally occurring amino acid residue is selected from the group consisting of 239D, 330L, 330Y and 332E, as numbered by the EU index as set forth in Kabat.

17. A method of reducing aggregation of an Fc variant protein comprising formulating said Fc variant protein in the liquid formulation of claim 1.

18. The method of claim 17, wherein the aggretion of an Fc variant protein is reduced by at least 10% compared to the aggregation when the same Fc variant is formulated in 10 mM Histidine pH 6.0.

19. A pre-lyophilization bulk formulation comprising an Fc variant protein at a concentration between 20 mg/mL and 100 mg/mL, 6% trehalose, 2% arginine (˜115 mM), 0.025% polysorbate-80 and 10 mM histidine buffer, wherein said formulation has a pH of between 6.0 and 6.5.

20. A liquid formulation comprising an Fc variant protein at a concentration between about 20 mg/mL and about 100 mg/mL, about 50 mM to about 300 mM citrate, and about 10% to about 20% trehalose and optionally about 0.001% to about 0.1% polysorbate, wherein said formulation has a pH of between 6.0 and 6.5.

21-29. (canceled)


SEQUENCE LISTING (20 sequences)

SEQ ID NO: 1 - DNA, 361 bp, Artificial
  recombinant antibody region
gaggtgcagctggtggagtctgggggaggtgtggtacggcctggggggtccctgagactctcctgtgcagcctctgggttcaccgtcagtgattactccatgaactgggtccgccaggctccagggaagggcctggagtggattgggtttattagaaacaaagctaatgcctacacaacagagtacagtgcatctgtgaagggtagattcaccatctcaagagatgattcaaaaaacacgctgtatctgcaaatgaacagcctgaaaaccgaggacacagccgtgtattactgtaccacataccctaggtatcatgctatggactcctggggccagggcaccatggtcaccgtctcctcag

SEQ ID NO: 2 - protein, 120 aa, Artificial
  recombinant antibody region
EVQLVESGGGVVRPGGSLRLSCAASGFTVSDYSMNWVRQAPGKGLEWIGFIRNKANAYTTEYSASVKGRFTISRDDSKNTLYLQMNSLKTEDTAVYYCTTYPRYHAMDSWGQGTMVTVSS

SEQ ID NO: 3 - DNA, 321 bp, Artificial
  recombinant antibody region
gccatccagttgactcagtctccatcctccctgtctgcatctgtaggagacagagtcaccatcacttgcagggccagccaaagtattagcaacaacctacactggtacctgcagaagccagggcagtctccacagctcctgatctattatggcttccagtccatctctggggtcccatcaaggttcagtggcagtggatctgggacagatttcactctcaccatcagcagtctgcaacctgaagattttgcaacttactactgtcaacaggccaacagctggccgctcacgttcggcggagggaccaagctggagatcaaa

SEQ ID NO: 4 - protein, 107 aa, Artificial
  recombinant antibody region
AIQLTQSPSSLSASVGDRVTITCRASQSISNNLHWYLQKPGQSPQLLIYYGFQSISGVPSRFSGSGSGTDFTLTISSLQPEDFATYYCQQANSWPLTFGGGTKLEIK

SEQ ID NO: 5 - DNA, 351 bp, Artificial
  recombinant antibody region
caggtgcagctggtggagtctgggggaggcgttgtgcagcctggaaggtccctgagactctcctgtgcagcctctggattcaccttcagtagctatgacatgtcttgggttcgccaggctccgggcaagggtctggagtgggtcgcaaaagttagtagtggtggtggtagcacctactatttagacactgtgcagggccgattcaccatctccagagacaatagtaagaacaccctatacctgcaaatgaactctctgagagccgaggacacagccgtgtattactgtgcaagacatctgcatggcagttttgcttcttggggccaagggactacagtgactgtttctagt

SEQ ID NO: 6 - protein, 117 aa, Artificial
  recombinant antibody region
QVQLVESGGGVVQPGRSLRLSCAASGFTFSSYDMSWVRQAPGKGLEWVAKVSSGGGSTYYLDTVQGRFTISRDNSKNTLYLQMNSLRAEDTAVYYCARHLHGSFASWGQGTTVTVSS

SEQ ID NO: 7 - DNA, 321 bp, Artificial
  recombinant antibody region
gagattgtgctaactcagtctccagccaccctgtctctcagcccaggagaaagggcgactctttcctgccaggccagccaaagtattagcaacttcctacactggtatcaacaaaggcctggtcaagccccaaggcttctcatccgctatcgttcccagtccatctctgggatccccgccaggttcagtggcagtggatcagggacagatttcaccctcactatctccagtctggagcctgaagattttgcagtctattactgtcaacagagtggcagctggcctctgacgttcggaggggggaccaaggtggaaattaag

SEQ ID NO: 8 - protein, 107 aa, Artificial
  recombinant antibody region
EIVLTQSPATLSLSPGERATLSCQASQSISNFLHWYQQRPGQAPRLLIRYRSQSISGIPARFSGSGSGTDFTLTISSLEPEDFAVYYCQQSGSWPLTFGGGTKVEIK

SEQ ID NO: 9 - protein, 5 aa, Artificial
  recombinant antibody region
DYSMN

SEQ ID NO: 10 - protein, 19 aa, Artificial
  recombinant antibody region
FIRNKANAYTTEYSASVKG

SEQ ID NO: 11 - protein, 9 aa, Artificial
  recombinant antibody region
YPRYHAMDS

SEQ ID NO: 12 - protein, 11 aa, Artificial
  recombinant antibody region
RASQSISNNLH

SEQ ID NO: 13 - protein, 7 aa, Artificial
  recombinant antibody region
YGFQSIS

SEQ ID NO: 14 - protein, 9 aa, Artificial
  recombinant antibody region
QQANSWPLT

SEQ ID NO: 15 - protein, 5 aa, Artificial
  recombinant antibody region
SYDMS

SEQ ID NO: 16 - protein, 17 aa, Artificial
  recombinant antibody region
KVSSGGGSTYYLDTVQG

SEQ ID NO: 17 - protein, 8 aa, Artificial
  recombinant antibody region
HLHGSFAS

SEQ ID NO: 18 - protein, 8 aa, Artificial
  recombinant antibody region
QSISNFLH

SEQ ID NO: 19 - protein, 7 aa, Artificial
  recombinant antibody region
YRSQSIS

SEQ ID NO: 20 - protein, 9 aa, Artificial
  recombinant antibody region
QQSGSWPLT